(19) United States
(12) Patent Application Publication
(10) Pub. No.: US 2024/0382626 A1
(43) Pub. Date: Nov. 21, 2024

(54) IRRADIATION AMENABLE LIPOSOMAL DYE AGGREGATES AND METHODS OF SYNTHESIS AND USE THEREOF

(71) Applicant: Board of Regents, The University of Texas System, Austin, TX (US)

(72) Inventors: Tyrone Porter, Austin, TX (US); Noah Stern, Austin, TX (US); James Tunnell, Austin, TX (US); Binita Shrestha, Austin, TX (US)

(21) Appl. No.: 18/665,948

(22) Filed: May 16, 2024

Related U.S. Application Data
(60) Provisional application No. 63/502,521, filed on May 16, 2023.

(51) Int. Cl. A61K 49/00; A61K 41/00; A61K 47/68

(52) U.S. Cl. CPC A61K 49/0084; A61K 41/0057; A61K 47/6851; A61K 49/0032; A61K 49/0034

(57) ABSTRACT

The present invention provides lipid nanoparticles that are amenable to an irradiation at a wavelength at or above 850 nm, have an absorption peak from about 850 to 1100 nm wavelength, and comprise a high transition temperature lipid and a dye (e.g., a J-aggregate of a dye). In some embodiments, the dye (e.g., J-aggregate of the dye) is encapsulated in the lipid nanoparticle. In various embodiments, the present invention also relates to compositions comprising said lipid nanoparticles and methods of generating said lipid nanoparticles and compositions thereof. The present invention further relates to methods relating to the said lipid nanoparticles for imaging, detection, and treatment of diseases or disorders (e.g., phototherapy) in a subject.

### CROSS-REFERENCE TO RELATED APPLICATIONS

The present application claims priority to and the benefit of U.S. Provisional Application No. 63/502,521, filed May 16, 2023, the disclosure of which is hereby incorporated by reference herein in its entirety.

### BACKGROUND OF THE INVENTION

Photoacoustic Imaging (PAI) has emerged as a promising non-invasive clinical imaging technique (Xu, M. et al., 2006, Review of Scientific Instruments, 77:041101). By leveraging the photoacoustic (PA) effect, PAI instruments detect the acoustic waves generated following the absorption of electromagnetic energy (Rosencwaig, A. et al., 1976, Journal of Applied Physics, 47:64-69). Utilizing the PA effect allows researchers and clinicians to combine the low attenuation of ultrasound waves and the high contrast of optical absorption from contrast agents into one versatile imaging system. Typically, these systems use non-ionizing pulsed lasers in the first near-infrared (NIR) window—600 to 1000 nm—due to the limited optical attenuation from biological components, such as hemoglobin and water, in this range. In this NIR window, the two major absorbers are oxygenated (HbO₂) and deoxygenated hemoglobin (HHb). With known profiles of the two forms of hemoglobin, fast imaging systems can give real-time estimates of blood oxygenation saturation (sO₂) in an area of interest. This simple, non-invasive, and contrast agent free technique for monitoring SO₂ has emerged as one of the most active areas of PAI research (Li, M. et al., 2018, Photoacoustics, 10:65-73; Chen, M et al., 2019, Opt. Lett., OL 44:3773-3776; Cao, F. et al., 2017, Applied Sciences, 7:1262; Karmacharya, M. B. et al., 2020, Diagnostics 10: 705).

In addition to imaging, many materials that respond favorably to the PA effect can also be leveraged for photothermal therapy (PTT). When pulsed lasers are replaced with continuous laser sources, highly localized heating can be achieved in biological tissues. Researchers have utilized nanoparticles that accumulate in solid tumors and efficiently absorb NIR wavelengths to facilitate PTT of solid tumors (Han, H. S., 2021, Biomedicines, 9:305; Bian, W. et al., 2021, ACS Appl. Nano Mater., 4:11353). However, the vast majority of nanoparticles used in these studies were not biodegradable and had poor photostability, which limits clinical translation.

With the use of high frequency ultrasound transducers, NIR pulsed lasers, and, if desired, exogenous contrast agents, PAI systems have tunable micron-level spatial resolutions up to several centimeters deep in biological tissues (Beard, P., 2011, Interface Focus 1:602-631; Smith, A. M et al., 2009, Nat Nanotechnol 4:710-711; Cho, E. C. et al., 2010, Trends in Molecular Medicine 16:561-573; Hu, S. & Wang, L. V., 2010, JBO 15:011101). With the growing availability of clinically inspired photoacoustic imaging systems combined with ultrasound systems and commercial spectral unmixing algorithms, the next major hurdle for further clinical use of PAI is the availability of more versatile exogenous contrast agents.

The most commonly used exogenous agents for PAI or PTT consist of a variety of metallic (Han, S et al., Biomed Opt Express, 10:3472-3483; Knights, O. B et al., Nanoscale Adv., 1:1472-1481; Chen, H et al., 2008, Langmuir 24:5233-5237; Han, Y et al., 2022, ACS Nano, 16:19053-19066; Min, K. H. et al., 2017, Theranostics, 7:4240-4254; Abdolahinia, E. D. et al., 2019, Life Sciences 231:116545; Li, W. & Chen, X., 2015, Nanomedicine (Lond), 10:299-320) and polymer-based (Cui, L. & Rao, J., 2017, Wiley Interdiscip Rev Nanomed Nanobiotechnol, 9; Pu, K et al., 2014, Nature Nanotech, 9:233-239; Liu, K et al., 2017, European Polymer Journal, 88:713-723) nanoparticles and several different small organic molecules (Wu, M et al., 2022, Advanced Healthcare Materials, 11:2201640; Zhang, W et al., 2022, Anal. Chem., 94:9697-9705; Bhattacharyya, S et al., 2008, Bioconjug Chem, 19:1186-1193; Laramie, M. D. et al., 2018, Molecules, 23:2766). Indocyanine green (IcG), an organic dye, is a very attractive option as it is a relatively cheap, small, Food and Drug Administration (FDA)-approved molecule with spectral characteristics active in the center of the first NIR window. As such, IcG has seen widespread use as a photoacoustic contrast agent (Chaudhary, Z et al., 2019, Biomater. Sci., 7:5002-5015; Hannah, A et al., 2014, ACS Nano, 8:250-259; Kim, G et al., 2007, JBO, 12:044020; Wang, G et al., 2016, ACS Appl. Mater. Interfaces, 8:5608-5617; Miyata, A et al., 2014, PLOS ONE, 9:e112667). With peak absorbance near 810 nm in blood, IcG has also recently been utilized for photothermal therapy with commonly available 808 nm lasers (Weng, Y. et al., 2022, Am. J. Transl. Res., 14:1991; An, F. et al., 2020, Journal of Nanobiotechnology, 18:49; Shirata, C. et al., 2017, Sci. Rep., 7:13958).

While promising, IcG has a very short circulation time, is known to photodegrade quickly following laser exposure, and has an absorption profile that directly overlaps with the spectra of oxygenated and deoxygenated hemoglobin. Circulation time and photodegradation can be improved via encapsulation within or binding to nanoparticles like liposomes, micelles, and silica constructs (Yan, F et al., 2016, Journal of Controlled Release, 224:217-228; Yaseen, M. A. et al., 2009, B. Mol. Pharmaceutics, 6:1321-1332). However, the spectral overlap provides its own challenge as at relevant concentrations IcG can obscure SO₂ measurement and estimation at physiologically relevant conditions (Baek, H. Y. et al., 2015, Korean J Anesthesiol, 68:122-127; Gehrung, M et al., 2019, JBO 24:121908). In cases where IcG is administered as a contrast agent, the hallmark ability of PA systems to measure SO₂ becomes much more complicated.

IcG is known to have a broad absorption profile that spans about a hundred nanometers with concentration dependent peaks around 710 nm and 780 nm. However, this spectral profile can be changed through the process of J-aggregation. Under the right conditions of temperature and concentration, stable aggregates of IcG called IcG J-aggregates (IcG-JA) can be formed (Rotermund, F et al., 1997, Chemical Physics, 220:385-392). IcG-JA have a stronger, narrower absorption peak redshifted deeper into the NIR at 890 nm. This offers a few benefits, namely fewer particles are needed to generate sufficient signal, a red-shifted wavelength indicates they may be effective at greater depths than IcG, and most importantly the 890 nm peak is shifted away from wavelengths typically used to identify hemoglobin content in blood.

In the first NIR window, this 890 nm peak is relatively unique as few contrast agents in this window have peaks above 850 nm. Alone IcG-JA are promising contrast agents (Liu, R et al, 2017, Nanotheranostics, 1:430-439), but a few instances have taken to combining them with nanoparticles for further functionalization (Changalvaie, B, et al., 2019, ACS Appl. Mater. Interfaces, 11:46437-46450; Wood, C. A. et al., 2021, Nat Commun, 12:5410; Cheung, C. C. L. et al., 2020, Nanotheranostics, 4:91-106). Additionally, IcG-JA encapsulation techniques also struggle with inconsistent particles, low encapsulation efficiencies, and long synthesis times.

Thus, there is a need in the art for nanoparticles that can encapsulate a dye, including a J-Aggregates of a dye, and be amenable to an irradiation at a wavelength of at or above about 850 nm as well as effective methods of producing thereof. The present invention addresses this need.

### BRIEF SUMMARY OF THE INVENTION

In one aspect, the present invention relates to a composition comprising a lipid nanoparticle and a dye, wherein the dye is a J-aggregate of the dye encapsulated in the lipid nanoparticle, wherein the lipid nanoparticle comprises a high transition temperature lipid having a melting temperature of above about 60° C., has an absorption peak from about 850 nm to about 1100 nm wavelength, and is amenable to an irradiation at a wavelength of at or above about 850 nm and wherein the composition is a transition metal-free composition.

In one embodiment, the lipid nanoparticle has an absorption peak from about 885 nm to about 895 nm wavelength.

In one embodiment, the lipid nanoparticle is amenable to an irradiation at a wavelength of between about 885 nm to about 895 nm wavelength.

In one embodiment, the high transition temperature lipid is selected from the group consisting of 1,2-diarachidoyl-sn-glycero-3-phosphocholine (20:0 PC), 1,2-dilauroyl-sn-glycero-3-phosphocholine (12:0 PC), 1,2-ditridecanoyl-sn-glycero-3-phosphocholine (13:0 PC), 1,2-dimyristoyl-sn-glycero-3-phosphocholine (14:0 PC), 1,2-dipentadecanoyl-sn-glycero-3-phosphocholine (15:0 PC), 1,2-dipalmitoyl-sn-glycero-3-phosphocholine (16:0 PC), 1,2-diheptadecanoyl-sn-glycero-3-phosphocholine (17:0 PC), 1,2-distearoyl-sn-glycero-3-phosphocholine (18:0 PC or DSPC), 1,2-dinonadecanoyl-sn-glycero-3-phosphocholine (19:0 PC), 1,2-diheneicosanoyl-sn-glycero-3-phosphocholine (21:0 PC), 1,2-dibehenoyl-sn-glycero-3-phosphocholine (22:0 PC), 1,2-ditricosanoyl-sn-glycero-3-phosphocholine (23:0 PC), 1,2-dilignoceroyl-sn-glycero-3-phosphocholine (24:0 PC), 1,2-diheptadecanoyl-sn-glycero-3-phospho-L-serine (17:0 PS), 1,2-distearoyl-sn-glycero-3-phospho-L-serine (18:0 PS), 1,2-dipalmitoyl-sn-glycero-3-phosphate (16:0 PA), 1,2-diheptadecanoyl-sn-glycero-3-phosphate (17:0 PA), 1,2-distearoyl-sn-glycero-3-phosphate (18:0 PA), 1,2-dipalmitoyl-sn-glycero-3-phosphoethanolamine (16:0 PE), 1,2-distearoyl-sn-glycero-3-phosphoethanolamine (18:0 PE), 1,2-diarachidoyl-sn-glycero-3-phosphoethanolamine (20:0 PE), and any combination thereof.

In one embodiment, the J-aggregate of the dye is a J-aggregate of a cyanine dye. In one embodiment, the J-aggregate of the dye is an indocyanine green J aggregate (ICGJ).

In one embodiment, the high transition temperature lipid is 1,2-diarachidoyl-sn-glycero-3-phosphocholine (20:0 PC) and the J-aggregate of the dye is an indocyanine green J aggregate (ICGJ).

In one embodiment, the lipid nanoparticle has an average hydrodynamic diameter of about 100 nm to about 200 nm.

In one embodiment, the lipid nanoparticle is a lipid vesicle. In one embodiment, the lipid vesicle is a liposome.

In one embodiment, the composition further comprises a polymer. In one embodiment, the polymer is selected from the group consisting of a polyethylene glycol (PEG), 1,2-distearoyl-sn-glycero-3-phosphoethanolamine-polyethylene glycol (DSPE-PEG), 1,2-distearoyl-sn-glycero-3-phosphoethanolamine-N-[amino(polyethylene glycol)-2000] (DSPE-PEG(2000)-amine), and any combination thereof.

In one embodiment, the composition further comprises a second lipid. In one embodiment, the second lipid is cholesterol.

In one embodiment, the molar ratio of the high transition temperature lipid to the polymer to the second lipid is from about 80:30:10 to about 90:50:15. In one embodiment, the molar ratio of the high transition temperature lipid to the polymer is about 85:50:15 to about 85:50:20.

In one embodiment, the composition comprises a targeting domain attached to the surface of the lipid nanoparticle. In one embodiment, the targeting domain binds to at least one cancer cell. In one embodiment, the targeting domain is selected from the group consisting of an antibody, an antibody fragment, a peptide sequence, aptamer, a ligand, a gene component, and any combination thereof. In one embodiment, the targeting domain binds to a cancer antigen.

In one embodiment, the composition further comprises a therapeutic agent. In one embodiment, the therapeutic agent is attached to the surface of the lipid nanoparticle. In one embodiment, the therapeutic agent is a chemotherapeutic agent.

In one embodiment, a contrast agent comprises the composition and a pharmaceutically acceptable excipient.

In one embodiment, the contrast agent is used in an imaging technique selected from the group consisting of: photoacoustic imaging, thermal imaging, photothermal imaging, and any combination thereof.

In another aspect, the present invention provides a method of generating the composition wherein the method comprises the steps of: generating a lipid nanoparticle, wherein the lipid nanoparticle comprises a high transition temperature lipid having a melting temperature of above about 60° C., adding a dye, encapsulating the dye in the lipid nanoparticle, and heating the dye encapsulated in the lipid nanoparticle to generate a J-aggregate of the dye encapsulated by the lipid nanoparticle.

In one embodiment, the step of generating a lipid nanoparticle comprises mixing the high transition temperature lipid with a polymer and a second lipid. In one embodiment, the molar ratio of the high transition temperature lipid to the polymer to the second lipid is from about 80:30:10 to about 90:50:15. In one embodiment, the molar ratio of the high transition temperature lipid to the polymer is about 85:50:15.

In one embodiment, the polymer is selected from the group consisting of a polyethylene glycol (PEG), 1,2-distearoyl-sn-glycero-3-phosphoethanolamine-polyethylene glycol (DSPE-PEG), 1,2-distearoyl-sn-glycero-3-phosphoethanolamine-N-[amino(polyethylene glycol)-2000] (DSPE-PEG(2000)-amine), 1,2-distearoyl-sn-glycero-3-phosphoethanolamine PEG maleimide (DSPE-PEG-maleimide), DSPE-PEG-carboxy-N-hydroxysuccinimide (NHS), DSPE-PEG-folate, DSPE-PEG-biotin, and any combination thereof.

In one embodiment, the second lipid is cholesterol. In one embodiment, the second lipid is subsequently removed.

In one embodiment, the step of adding a dye comprises mixing the dye and the lipid nanoparticle at a temperature of between about 60° C. to about 85° C. In one embodiment, the temperature is about 80° C.

In one embodiment, the step of heating the dye encapsulated in the lipid nanoparticle to generate a J-aggregate of the dye encapsulated by the lipid nanoparticle heating the dye encapsulated in the lipid nanoparticle at a temperature of between about 60° C. for between about 14 hr to about 30 hr.

In another aspect, the present invention provides an imaging method, comprising the steps of contacting a biological tissue with the composition, applying energy to a biological tissue comprising the composition, and imaging the biological tissue comprising the composition.

In one embodiment, imaging the biological tissue comprises application of an imaging technique selected from the group consisting of: photoacoustic imaging, thermal imaging, photothermal imaging, and any combination thereof.

In one embodiment, the biological tissue is a cancer cell. In one embodiment, the cancer cell is present in a mammal.

In one embodiment, applying energy to the biological tissue comprises application of at least one selected from the group consisting of: exposing the biological tissue to irradiation at a wavelength between about 850 to about 1100 nm, irradiating at least a portion of the biological tissue with a light source, applying a radio frequency field, and any combination thereof. In one embodiment, imaging the biological tissue comprises transducing the resulting ultrasound signal from the biological tissue and producing an image in a data processor from the transduced ultrasound signal.

In one embodiment, the composition further comprises a targeting domain.

In one embodiment, the method further comprises the step of allowing the composition to accumulate in a region of the biological tissue, wherein the targeting domain facilitated accumulation of the composition in the region. In one embodiment, the composition further comprises at least one therapeutic agent. In one embodiment, the composition accumulates around a region of biological tissue.

In another aspect, the present invention provides a method of treating a disease or disorder in a subject in need thereof, wherein the method comprises the step of administering a therapeutically effective amount of the composition to the subject.

In one embodiment, the method further comprises irradiating the subject at a wavelength of at or above about 850 nm. In one embodiment, the method comprises irradiating the subject at a wavelength of between about 885 nm to about 895 nm wavelength.

In one embodiment, the irradiation selectively kills or inhibits at least one cell of interest. In one embodiment, the at least one cell of interest is a cancer cell. In one embodiment, the disease or disorder is a cancer. In one embodiment, the cancer is selected from the group consisting of a brain cancer, head and neck cancer, breast cancer, prostate cancer, liver cancer, skin cancer, and any combination thereof. In one embodiment, the composition further comprises a targeting domain.

In one embodiment, the method further comprises the step of allowing the composition to accumulate in at least one cell of interest, wherein the targeting domain facilitated accumulation of the composition in the at least one cell of interest. In one embodiment, the composition accumulates around a region of biological tissue. In one embodiment, the composition accumulates around at least one cell of interest. In one embodiment, the composition accumulates in the blood stream around a region of biological tissue.

In one embodiment, the composition further comprises at least one therapeutic agent.

In another aspect, the present invention provides a phototherapy method, comprising the steps of: administering a therapeutically effective amount of the composition to the subject, and irradiating the subject at a wavelength of at or above about 850 nm.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following detailed description of various embodiments of the invention will be better understood when read in conjunction with the appended drawings. For the purpose of illustrating the invention, there are shown in the drawings illustrative embodiments. It should be understood, however, that the invention is not limited to the precise arrangements and instrumentalities of the embodiments shown in the drawings.

FIG. 1 depicts a schematic representation of the major steps of the liposomal J-Aggregates (L-JA) procedure; IcG hydration of a thin film containing high-transition temperature lipids followed by extrusion to size, dialysis to remove unencapsulated IcG, heating to form J-Aggregates within liposomes, and sterile filtration.

FIG. 2, comprising FIG. 2A through FIG. 2G, depicts representative photothermal data using a 808 nm laser. J-Aggregates of IcG and their liposomal form can repeatedly reach temperatures of 60° C. and above, while IcG can only be heated once. FIG. 2A depicts heating with a 808 nm laser. IcG, IcG-JA, and L-JA can all reach temperatures in excess of 60° C. over the course of about 20 seconds. FIG. 2B depicts 808 nm laser heating and highlights that IcG cannot be repeatedly heated. FIG. 2C depicts 852 nm laser heating. Unlike heating with a 808 nm laser, at equal concentrations the L-JA solution reaches significantly higher temperatures than IcG. Like with the 808 nm laser, when heated with the 852 nm laser, L-JA solutions can reach temperatures in excess of 60° C. rapidly and repeatedly. FIG. 2D depicts complementary heating data for FIG. 2A. FIG. 2E depicts cyclical heating. FIG. 2F depicts complementary heating data for FIG. 2C. FIG. 2G depicts images of cell death within the illumination area of the laser.

FIG. 3, comprising FIG. 3A through 3C, depicts representative optical data of 1,2-distearoyl-sn-glycero-3-phosphocholine (18:0 PC or DSPC) vs. 1,2-diarachidoyl-sn-glycero-3-phosphocholine (20:0 PC). FIG. 3A depicts an additional peak in the spectrum of DSPC at 780 nm represents incomplete aggregation and leakage of IcG from the particles. FIG. 3B depicts representative optical data of “extrusion only” samples which did not undergo a 20-hour heating, similar to DSPC particles in FIG. 3A. Extrusion only samples are high variable and often leave unaggregated IcG. FIG. 3C depicts optical data of lipids that are heated above the transition temperature for too long, where very little signal is observed at 890 nm (red line). Similar data is observed when DSPC particles are heated at the optimal temperature (60 to 65° C.) for the 16+ hours required to complete aggregation, which may be caused by complete lipid breakdown which obscures IcG in solution.

FIG. 4 depicts a schematic representative of the major steps of the L-JA procedure with additional adjustments performed for synthetic optimization.

FIG. 5 depicts representative results demonstrating photoacoustic properties of free IcG relative to IcG J-Aggregates (IcG-JA) and Liposomal J-Aggregates (L-JA).

FIG. 6, comprising FIG. 6A through FIG. 6D, depicts representative results demonstrating the particle characterization. FIG. 6A depicts representative size measurements obtained from DLS and NanoSight systems. Average size from dynamic light scattering (DLS) and NanoSight align well. FIG. 6B depicts a representative scanning transmission electron microscopy (STEM) image of stained L-JA. FIG. 6C depicts representative encapsulation efficiency sample calculations. Final EE % obtained as a ratio of peak signals of final particles to rehydration solution. FIG. 6D depicts representative results demonstrating surface (Zeta) potential of IcG J-Aggregates (IcG-JA), Liposomal J-Aggregates (L-JA), and Liposomal IcG (L-IcG).

FIG. 7, comprising FIG. 7A and FIG. 7B, depicts representative results demonstrating photoacoustic properties of the L-JA particles. FIG. 7A depicts representative results demonstrating spectral profiles of L-JA (dark green), IcG-JA (violet), and IcG (light green) in 100% fetal bovine serum (FBS) at 37° C. At equal concentrations, J-Aggregates have a peak signal nearly 3 times larger than non-aggregated forms and have a narrower redshifted peak in the NIR. FIG. 7B depicts representative PA images of L-JA (left), IcG (center), and IcG-JA (right) in tubes embedded within tissue mimicking phantoms at 710 nm (top), 780 nm (center), and 890 nm (bottom). J-Aggregate particles have peak signals at 890 nm and non-aggregate forms have a broader spectral profile with notable peaks at 710 nm and 780 nm.

FIG. 8 depicts representative results demonstrating long-term stability of liposomes and free J-Aggregates in PBS and FBS buffer solutions. IcG is known to undergo degradation by serum proteins. IcG J-Aggregates will also experience some degradation when exposed to serum and the liposomal shell prevents degradation as the serum proteins are unable to bind to the IcG, degrading its signal.

FIG. 9, comprising FIG. 9A and FIG. 9B, depicts representative results demonstrating photostability of L-JA and IcG. FIG. 9A depicts representative results demonstrating PA intensity of L-JA (top, dark green) and IcG (bottom, light green) during 30 minutes of imaging using 800 nm laser pulses. IcG loses more than 50% of signal amplitude after just over 5 minutes and fully quenches after 25 minutes. L-JA signal reduces slightly over time, but does not drop by more than 50% during 30 minutes of constant imaging. FIG. 9B depicts representative results demonstrating average signals from L-JA and IcG during 5-minute segments from the imaging session. L-JA remains clearly visible throughout imaging while IcG loses signal rapidly.

FIG. 10, comprising FIG. 10A through FIG. 10C, depicts representative results of cytotoxicity experiments. FIG. 10A depicts representative results demonstrating cell viability following 24 hr incubation of L-JA, IcG-JA, or IcG with HEK-293T cells. FIG. 10B depicts representative results demonstrating cell viability following 24 hr incubation of L-JA, IcG-JA, or IcG with HUVECs. L-JA have similar effects to IcG with significant cytotoxicity relative to control not occurring until a concentration of 0.2 mM (p<0.05). In vivo injections occur at a dose of 5 mg/kg which would lead to concentration values much lower than this 0.2 mM limit. FIG. 10C depicts concentration dependent cell death in response to laser illumination for liposomal nanoparticles and regular IcG at a wavelength of 852 nm. “Control” refers to cells that did not receive particles or laser illumination and “laser” refers to cells that did not receive particles but were illuminated with the laser. All laser exposures were for 5 minutes at a power density of 0.4 W/cm².

FIG. 11 depicts representative results demonstrating cytotoxicity data of liposomal J-Aggregates at relevant doses with HEK-293T cells, HUVECs, B16-F0 cells, and RAW cells.

FIG. 12, comprising FIG. 12A and FIG. 12B, depicts PTT efficacy of IcG and L-JA. FIG. 12A depicts heating of equal concentrations (˜0.1 mM) of IcG and L-JA at 808 nm and 852 nm as measured by thermal camera. At 808 nm, heating is relatively similar, but 852 nm, L-JA heat to a much larger extent. FIG. 12B depicts 852 nm heating of L-JA and IcG at multiple concentrations. L-JA reaches significantly higher temperatures than IcG, and is equally effective at a 20 times lower concentration.

FIG. 13, comprising FIG. 13A through FIG. 13D, depicts representations of the mouse kidney with different modalities. FIG. 13A depicts representative ultrasound B-Mode data. FIG. 13B depicts representative power doppler imaging. FIG. 13C depicts representative raw PA-OxyHemo data. FIG. 13D depicts representative raw PA-OxyHemo data limited to areas of high blood flow shown in FIG. 13B. Basing PA analysis on areas of high blood flow removes excess noise and ensures calculations are being performed in areas with sufficient blood flow.

FIG. 14, comprising FIG. 14A through FIG. 14F, depicts representative power-doppler image data of mice injected with a 5 mg/kg dose of contrast agent. FIG. 14A depicts representative SO₂ values for L-JA at varying time points following injection. L-JA does not cause a significant change in measured sO₂ across regions of interest (ROIs) and time points. FIG. 14B depicts representative SO₂ values for IcG at varying time points following injection. IcG does cause a significant drop in measured SO₂ during circulation (p<0.05). FIG. 14C depicts representative SO₂ values for IcG-JA at varying time points following injection. IcG-JA does not cause a significant change in measured SO₂ across ROIs and time points. FIG. 14D depicts representative sample images of drawn ROIs at each time point for L-JA injections. FIG. 14E depicts representative sample images of drawn ROIs at each time point for IcG injections. FIG. 14F depicts representative sample images of drawn ROIs at each time point for IcG-JA injections.

FIG. 15 depicts representative in vivo PA images monitoring sO₂ levels with injected IcG. A large drop in sO₂ occurs following injection and a slow climb back to baseline level occurs as IcG clears, which does not occur with IcG-JA or L-JA.

FIG. 16 depicts representative in vivo PA images monitoring SO₂ levels with injected L-JA.

FIG. 17 depicts representative in vitro photoacoustic images of free IcG, free J-Aggregates, and liposomal J-Aggregates and representative results showing high photostability of J-Aggregates at 800 nm.

FIG. 18, comprising FIG. 18A through FIG. 18F, depicts representative results demonstrating significant contrast enhancement from L-JA. FIG. 18A depicts representative results demonstrating contrast enhancement (Signal/Baseline) from unmixed contrast agent signals for L-JA at varying time points following injection. L-JA provides a significant contrast enhancement immediately following injection at 4 hours and 24 hours demonstrating its prolonged circulation time (p<0.05). FIG. 18B depicts representative results demonstrating contrast enhancement (Signal/Baseline) from unmixed contrast agent signals for IcG at varying time points following injection. FIG. 18C depicts representative results demonstrating contrast enhancement (Signal/Baseline) from unmixed contrast agent signals for IcG-JA at varying time points following injection. FIG. 18D depicts representative sample images of drawn ROIs at each time point for L-JA injections. FIG. 18E depicts representative sample images of drawn ROIs at each time point for IcG injections. IcG provides a significant contrast enhancement but is fully cleared over the course of a few minutes (p<0.05) and has a much lower overall signal change. Note that the colorbar for IcG has half the range of L-JA. FIG. 18F depicts representative sample images of drawn ROIs at each time point for IcG-JA injections.

FIG. 19 depicts representative in vivo PA images monitoring signals with injected J-Aggregates and display maps of where the L-JA are based on special algorithms that use the raw data from imaging at various wavelengths.

FIG. 20, comprising FIG. 20A and FIG. 20B, depicts representative photothermal data using a 808 nm laser. FIG. 20A depicts representative results of cyclic heating of liposomal J-Aggregates. Conversely, FIG. 2B shows that IcG cannot be repeatedly heated. FIG. 20B depicts representative results demonstrating rates of temperature changes during cyclic heating of liposomal J-Aggregates.

FIG. 21 are images from videos and depicts representative results demonstrating moving average percentages of monomeric IcG interference in sO₂ imaging. The top left depicts ultrasound, bottom left depicts oxy-hemo, and right depicts data taken from drawing an ROI.

FIG. 22 depicts representative results demonstrating optical properties of IcG J-Aggregates. J-Aggregation results in a narrow peak response redshifted deeper into the near-infrared region which offers distinct advantages for photoacoustic imaging.

FIG. 23 depicts representative results demonstrating reproducible characteristics of liposomal particles.

FIG. 24 depicts a schematic representation of how vascular normalization allows for more effective cancer therapies. When a balance of pro- and anti-angiogenic factors is maintained, small leaky vessels may be pruned allowing for improved delivery of oxygen and potentially therapeutics. Finding this normalization “therapeutic dose” is important as too low a dose will be ineffective and too high a does may prune vasculature too much leading to a more hypoxic and aggressive tumor.

FIG. 25 depicts representative non-invasive monitoring of tumor vasculature and optical properties of IcG J-Aggregates relative to IcG in water, IcG in Plasma, HbO₂, and HbR.

FIG. 26 depicts representative in vivo PA images of the kidney containing J-Aggregates.

### DETAILED DESCRIPTION

The present invention is based, in part, on the unexpected discovery of an effective and reproducible method for generating lipid nanoparticles comprising 1,2-diarachidoyl-sn-glycero-3-phosphocholine (20:0 PC) that encapsulated indocyanine green J-aggregate (ICGJ), were amenable to an irradiation at a wavelength at or above 850 nm, and had an absorption peak from about 850 to 1100 nm wavelength. Thus, in various embodiments, the present invention provides lipid nanoparticles that is amenable to an irradiation at a wavelength at or above 850 nm, has an absorption peak from about 850 to 1100 nm wavelength, and comprises a high transition temperature lipid and a dye (e.g., a J-aggregate of a dye, such as ICGJ). In some embodiments, the dye (e.g., J-aggregate of the dye, such as ICGJ) is encapsulated in the lipid nanoparticle. In some embodiments, the lipid nanoparticle forms liposome by encapsulating the dye (e.g., J-aggregate of the dye, such as ICGJ) in the lipid nanoparticle comprising a high transition temperature lipid. Thus, in some aspects, the invention provides compositions comprising ICGJ encapsulated in a lipid nanoparticle or liposome.

In some embodiments, the lipid nanoparticles comprise a targeting domain that directs the lipid nanoparticle to a specific cell or tissue (e.g. cancer cell). Thus, the present invention relates to compositions and methods relating to lipid nanoparticles that selectively target cells associated with diseases or disorders (e.g., cancer cells) and can be used for detection, imaging, and treatment of diseases or disorders (e.g., phototherapy) in a subject.

The present invention additionally provides methods of generating said lipid nanoparticles and compositions thereof.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

The term “about” will be understood by persons of ordinary skill in the art and will vary to some extent depending on the context in which it is used. As used herein when referring to a measurable value such as an amount, a temporal duration, and the like, the term “about” is meant to encompass variations of 20% or ±10%, more preferably ±5%, even more preferably ±1%, and still more preferably ±0.1% from the specified value, as such variations are appropriate to perform the disclosed methods.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

The term “biological tissue” as used herein refers to a collection of interconnected cells and extracellular matrix that perform a similar function or functions within an organism. Examples of biological tissues include, but are not limited to, connective tissue, muscle tissue, nervous tissue (of the brain, spinal cord, and nerves), epithelial tissue, organ tissue, cancer tissue, and any combination thereof. Connective tissue includes fibrous tissue like fascia, tendon, ligaments, heart valves, bone, and cartilage. Muscle tissue includes skeletal muscle tissue, smooth muscle tissue, such as esophageal, stomach, intestinal, bronchial, uterine, urethral, bladder, and blood vessel tissue, and cardiac muscle tissue. Epithelial tissue includes simple epithelial tissue, such as alveolar epithelial tissue, blood vessel endothelial tissue, and heart mesothelial tissue, and stratified epithelial tissue.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Cancer,” as used herein, refers to the abnormal growth or division of cells. Generally, the growth and/or life span of a cancer cell exceeds, and is not coordinated with, that of the normal cells and tissues around it. Cancers may be benign, pre-malignant or malignant. Cancer occurs in a variety of cells and tissues, including, but not limited to, the oral cavity (e.g., mouth, tongue, pharynx, etc.), digestive system (e.g., esophagus, stomach, small intestine, colon, rectum, liver, bile duct, gall bladder, pancreas, etc.), respiratory system (e.g., larynx, lung, bronchus, etc.), bones, joints, skin (e.g., basal cell, squamous cell, meningioma, etc.), breast, genital system, (e.g., uterus, ovary, prostate, testis, etc.), urinary system (e.g., bladder, kidney, ureter, etc.), eye, nervous system (e.g., brain, etc.), head and neck, endocrine system (e.g., thyroid, etc.), soft tissues (e.g., muscle, fat, etc.), and hematopoietic system (e.g., lymphoma, myeloma, leukemia, acute lymphocytic leukemia, chronic lymphocytic leukemia, acute myeloid leukemia, chronic myeloid leukemia, etc.).

As used herein, the term “diagnosis” refers to the determination of the presence of a disease or disorder. In some embodiments of the present invention, methods for making a diagnosis are provided which permit determination of the presence of a particular disease or disorder.

A “therapeutic” treatment is a treatment administered to a subject who exhibits signs or symptoms of a disease or disorder, for the purpose of diminishing or eliminating those signs or symptoms.

As used herein, “treating a disease or disorder” means reducing the severity and/or frequency with which a sign or symptom of the disease or disorder is experienced by a subject.

A disease or disorder is “alleviated” if the severity of a sign or symptom of the disease or disorder, the frequency with which such a sign or symptom is experienced by a subject, or both, is reduced.

The term “derivative” refers to a small molecule that differs in structure from the reference molecule, but retains the essential properties of the reference molecule. A derivative may change its interaction with certain other molecules relative to the reference molecule. A derivative molecule may also include a salt, an adduct, tautomer, isomer, or other variant of the reference molecule.

The term “tautomers” are constitutional isomers of organic compounds that readily interconvert by a chemical process (tautomerization).

The term “isomers” or “stereoisomers” refer to compounds, which have identical chemical constitution, but differ with regard to the arrangement of the atoms or groups in space.

The term “H-aggregate” as used herein refers to aggregates of monomers of a compound (e.g., a dye) whereupon aggregation causes a shift in the extinction to the lower wavelengths.

The term “J-aggregate” as used herein refers to aggregates of monomers of a compound (e.g., a dye) whereupon aggregation causes a shift in the extinction to higher wavelengths.

As used herein, the term “nanoparticle” refers to particles having a particle size on the nanometer scale, less than 1 micrometer. For example, the lipid nanoparticle may have a particle size up to about 50 nm. In another example, the lipid nanoparticle may have a particle size up to about 10 nm. In another example, the lipid nanoparticle may have a particle size up to about 6 nm. As used herein, “nanoparticle” refers to a number of nanoparticles, including, but not limited to, nanoclusters, nanocapsules, core-shell nanocapsules, nanovesicles, micelles, block copolymer micelles, lamaellae shaped particles, polymersomes, dendrimers, and other nano-size particles of various other small fabrications that are known to those of skill in the art. The shapes and compositions of nanoparticles may be guided during condensation of atoms by selectively favoring growth of particular crystal facets to produce spheres, rods, wires, discs, cages, core-shell structures and many other shapes. The definitions and understandings of the entities falling within the scope of nanocapsule are known to those of skill in the art, and such definitions are incorporated herein by reference and for the purposes of understanding the general nature of the subject matter of the present application. However, the following discussion is useful as a further understanding of some of these terms.

For example, the term “nanocapsule” refers to a vesicular system or hollow particle with a shell surrounding a core-forming space, which, in certain instances, can be used for transporting a payload on a nanoscale level. A nanocapsule may also be a nano-sized version of a container. The payload of the nanocapsule can be, but is not limited to drugs, medicaments, pharmaceutical compositions, chemical compositions, therapeutic compositions, biological macromolecules, dyes, biological material, immunological compositions, nutritional compositions, vitamins, proteins, nucleic acids, antibodies and vaccines. Various materials may be used for producing such nanocapsules. Nanocapsule refers to a particle having a hollow core that is surrounded by a shell, such that the particle has a size of less than about 1000 nanometers. When a nanocapsule includes a bioactive component, the bioactive component is located in the core that is surrounded by the shell of the nanocapsule.

As used herein, the term “nanocage” refers to a nanocapsule, whereby the shell is not solid, as described for the nanocapsule, but has multiple holes or pores in its shell, thereby making it possible for the payload within the core of the nanocage to come into contact with the surrounding environment. These holes or pores may be regular or irregular in shape and/or spacing on the surface of the particle.

The term “micelle”, a useful article in the employment of a general aspect of the present invention, can generally be thought of as a small—on the order of usually nanometers in diameter—aggregate of amphiphilic linear molecules having a polar, or hydrophilic end and an opposite non-polar, or hydrophobic end. These linear molecules can be comprised of simple molecules, or polymeric chains. A micelle can also be referred to as an aggregate of surfactant molecules dispersed in a liquid colloid. A typical micelle in aqueous solution can form an aggregate with the hydrophilic “head” regions in contact with surrounding solvent, and the sequestering of the hydrophobic tail regions in the micelle center. Other and similar definitions, descriptions and understandings of micelles are also known to those of skill in the art and are incorporated herein by reference.

The term “liposome” as used herein refers to an artificially prepared vesicle composed of a lipid bilayer. A liposome may be classified as a unilamellar vesicle or a multivesicular vesicle.

The term “lipid bilayer” as used herein refers to a membrane made of two layers of lipid molecules. The lipid bilayer may have a similar thickness as that of a naturally existing bilayer, such as a cell membrane, a nuclear membrane, and a virus envelope. For example, the lipid bilayer may have a thickness of about 10 nm or less, for example, in a range of about 1 nm to about 9 nm, about 2 nm to about 8 nm, about 2 nm to about 6 nm, about 2 nm to about 4 nm, or about 2.5 nm to about 3.5 nm. The lipid bilayer is a barrier that retains ions, proteins, and other molecules while also preventing them from diffusing into undesirable areas. The “lipid molecules” forming the lipid bilayer may comprise a molecule including a hydrophilic head and a hydrophobic tail. The lipid molecule may comprise from about 14 to about 50 carbon atoms. Examples of the lipid molecules which may form a lipid bilayer include phospholipids, lipids conjugated to polyethylene glycol (PEG), cholesterol, or any combination thereof.

“Dendrimers” have descriptions, definitions and understandings in the literature. For example, and without limitation and including other and similar definitions, descriptions and understandings in the art, the term dendrimer from the Greek word, “dendron”, for tree, can refer to a synthetic, three-dimensional molecule with branching parts. Descriptions and understandings of dendrimers can be gleaned from Holister et al., Dendrimers, Technology White Papers nr. 6, pub. October 2003 by cientifica, as well as the other literature published by those skilled in the art on dendrimers, all of which are incorporated herein by reference.

The definitions, descriptions and understandings of “nanovesicle” are well known to those of skill in the art, and are incorporated herein by reference. For example, “nanovesicle” can refer to a variety of small sac, sac-like or globular structures capable of containing fluid or other material therein

“Pharmaceutically acceptable” refers to those properties and/or substances which are acceptable to the subject from a pharmacological/toxicological point of view and to the manufacturing pharmaceutical chemist from a physical/chemical point of view regarding composition, formulation, stability, subject acceptance and bioavailability. “Pharmaceutically acceptable carrier” refers to a medium that does not interfere with the effectiveness of the biological activity of the active ingredient(s) and is not toxic to the host to which it is administered.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the subject. Some examples of materials that may serve as pharmaceutically acceptable carriers include: sugars, such as lactose, glucose, and sucrose; starches, such as corn starch and potato starch; cellulose, and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the subject. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art.

The term “pharmaceutically acceptable salt” refers to any pharmaceutically acceptable salt, which upon administration to the subject is capable of providing (directly or indirectly) a compound as described herein. Such salts preferably are acid addition salts with physiologically acceptable organic or inorganic acids. Examples of the acid addition salts include mineral acid addition salts such as, for example, hydrochloride, hydrobromide, hydroiodide, sulphate, nitrate, phosphate, and organic acid addition salts such as, for example, acetate, trifluoroacetate, maleate, fumarate, citrate, oxalate, succinate, tartrate, malate, mandelate, methane sulphonate, and p-toluenesulphonate. Examples of the alkali addition salts include inorganic salts such as, for example, sodium, potassium, calcium and ammonium salts, and organic alkali salts such as, for example, ethylenediamine, ethanolamine, N,N-dialkylenethanolamine, triethanolamine, and basic amino acids salts. However, it will be appreciated that non-pharmaceutically acceptable salts also fall within the scope of the invention since those may be useful in the preparation of pharmaceutically acceptable salts. Procedures for salt formation are conventional in the art.

The term “solvate” in accordance with this invention should be understood as meaning any form of the active compound in accordance with the invention in which the said compound is bonded by a non-covalent bond to another molecule (normally a polar solvent), including especially hydrates and alcoholates.

As used herein, the term “pharmaceutical composition” refers to a mixture of at least one compound of the invention with other chemical components and entities, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, oral, aerosol, parenteral, ophthalmic, pulmonary and topical administration.

As used herein, the terms “therapeutic compound”, “therapeutic agent”, “drug”, “active pharmaceutical”, and “active pharmaceutical ingredient” are used interchangeably to refer to chemical entities that display certain pharmacological effects in a body and are administered for such purpose. Non-limiting examples of therapeutic agents include, but are not limited to, hydrophilic therapeutic agents, hydrophobic therapeutic agents, antibiotics, antibodies, small molecules, anti-cancer agents, chemotherapeutic agents, immunomodulatory agents, RNA molecules, siRNA molecules, DNA molecules, gene editing agents, gene-silencing agents, CRISPR-associated agents (e.g., guide RNA molecules, endonucleases, and variants thereof), analgesics, vaccines, anticonvulsants; anti-diabetic agents, antifungal agents, antineoplastic agents, anti-parkinsonian agents, anti-rheumatic agents, appetite suppressants, biological response modifiers, cardiovascular agents, central nervous system stimulants, contraceptive agents, dietary supplements, vitamins, minerals, lipids, saccharides, metals, amino acids (and precursors), nucleic acids and precursors, contrast agents, diagnostic agents, dopamine receptor agonists, erectile dysfunction agents, fertility agents, gastrointestinal agents, hormones, immunomodulators, antihypercalcemia agents, mast cell stabilizers, muscle relaxants, nutritional agents, ophthalmic agents, osteoporosis agents, psychotherapeutic agents, parasympathomimetic agents, parasympatholytic agents, respiratory agents, sedative hypnotic agents, skin and mucous membrane agents, smoking cessation agents, steroids, sympatholytic agents, urinary tract agents, uterine relaxants, vaginal agents, vasodilator, anti-hypertensive, hyperthyroids, anti-hyperthyroids, anti-asthmatics and vertigo agents. In certain embodiments, the one or more therapeutic agents are water-soluble, poorly water-soluble drug or a drug with a low, medium or high melting point. The therapeutic agents may be provided with or without a stabilizing salt or salts.

Some examples of active ingredients suitable for use in the pharmaceutical formulations and methods of the present invention include: hydrophilic, lipophilic, amphiphilic or hydrophobic, and that can be solubilized, dispersed, or partially solubilized and dispersed, on or about the nanocluster. The active agent-nanocluster combination may be coated further to encapsulate the agent-nanocluster combination and may be directed to a target by functionalizing the nanocluster with, e.g., aptamers and/or antibodies. Alternatively, an active ingredient may also be provided separately from the solid pharmaceutical composition, such as for co-administration. Such active ingredients can be any compound or mixture of compounds having therapeutic or other value when administered to an animal, particularly to a mammal, such as drugs, nutrients, cosmeceuticals, nutraceuticals, diagnostic agents, nutritional agents, and the like. The active agents described herein may be found in their native state, however, they will generally be provided in the form of a salt. The active agents described herein include their isomers, analogs and derivatives.

As used herein, the terms “targeting domain”, “targeting moiety”, or “targeting group” are used interchangeably and refer to all molecules capable of specifically binding to a particular target molecule and forming a bound complex as described above. Thus, the ligand and its corresponding target molecule form a specific binding pair.

The term “antibody”, as used herein, refers to an immunoglobulin molecule which is able to specifically bind to a specific epitope of an antigen. Antibodies can be intact immunoglobulins derived from natural sources, or from recombinant sources and can be immunoreactive portions of intact immunoglobulins. The antibodies in the present invention may exist in a variety of forms including, for example, polyclonal antibodies, monoclonal antibodies, multiple chain antibodies, intact immunoglobulins, synthetic antibodies, recombinant antibodies, intracellular antibodies (“intrabodies”), Fv, Fab, Fab′, F(ab)2 and F(ab′)2, as well as single chain antibodies (scFv), heavy chain antibodies, such as camelid antibodies, and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antibody fragment” refers to at least one portion of an intact antibody, or recombinant variants thereof, and refers to the antigen binding domain, e.g., an antigenic determining variable region of an intact antibody, that is sufficient to confer recognition and specific binding of the antibody fragment to a target, such as an antigen.

By the term “synthetic antibody” as used herein, is meant an antibody which is generated using recombinant DNA technology, such as, for example, an antibody expressed by a bacteriophage. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

A “humanized antibody” refers to a type of engineered antibody having its CDRs derived from a non-human donor immunoglobulin, the remaining immunoglobulin-derived parts of the molecule being derived from one (or more) human immunoglobulin(s). In addition, framework support residues may be altered to preserve binding affinity (see, e.g., 1989, Queen et al., Proc. Natl. Acad Sci USA, 86:10029-10032; 1991, Hodgson et al., Bio/Technology, 9:421). A suitable human acceptor antibody may be one selected from a conventional database, e.g., the KABAT database, Los Alamos database, and Swiss Protein database, by homology to the nucleotide and amino acid sequences of the donor antibody. A human antibody characterized by a homology to the framework regions of the donor antibody (on an amino acid basis) may be suitable to provide a heavy chain constant region and/or a heavy chain variable framework region for insertion of the donor CDRs. A suitable acceptor antibody capable of donating light chain constant or variable framework regions may be selected in a similar manner. It should be noted that the acceptor antibody heavy and light chains are not required to originate from the same acceptor antibody. The prior art describes several ways of producing such humanized antibodies (see for example EP-A-0239400 and EP-A-054951).

A “chimeric antibody” refers to a type of engineered antibody which contains a naturally-occurring variable region (light chain and heavy chains) derived from a donor antibody in association with light and heavy chain constant regions derived from an acceptor antibody.

The term “donor antibody” refers to an antibody (monoclonal, and/or recombinant) which contributes the amino acid sequences of its variable regions, CDRs, or other functional fragments or analogs thereof to a first immunoglobulin partner, so as to provide the altered immunoglobulin coding region and resulting expressed altered antibody with the antigenic specificity and neutralizing activity characteristic of the donor antibody.

The term “acceptor antibody” refers to an antibody (monoclonal and/or recombinant) heterologous to the donor antibody, which contributes all (or any portion, but in some embodiments all) of the amino acid sequences encoding its heavy and/or light chain framework regions and/or its heavy and/or light chain constant regions to the first immunoglobulin partner. In certain embodiments a human antibody is the acceptor antibody.

By the term “recombinant antibody” as used herein, is meant an antibody which is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

An “antibody heavy chain,” as used herein, refers to the larger of the two types of polypeptide chains present in antibody molecules in their naturally occurring conformations, and which normally determines the class to which the antibody belongs.

An “antibody light chain,” as used herein, refers to the smaller of the two types of polypeptide chains present in antibody molecules in their naturally occurring conformations. Kappa (κ) and lambda (λ) light chains refer to the two major antibody light chain isotypes.

“CDRs” are defined as the complementarity determining region amino acid sequences of an antibody which are the hypervariable regions of immunoglobulin heavy and light chains. See, e.g., Kabat et al., Sequences of Proteins of Immunological Interest, 4^{th} Ed., U.S. Department of Health and Human Services, National Institutes of Health (1987). There are three heavy chain and three light chain CDRs (or CDR regions) in the variable portion of an immunoglobulin. Thus, “CDRs” as used herein refers to all three heavy chain CDRs, or all three light chain CDRs (or both all heavy and all light chain CDRs, if appropriate). The structure and protein folding of the antibody may mean that other residues are considered part of the antigen binding region and would be understood to be so by a skilled person. See for example Chothia et al., (1989) Conformations of immunoglobulin hypervariable regions; Nature 342, p 877-883.

The terms “effective amount” and “pharmaceutically effective amount” refer to a sufficient amount of an agent to provide the desired biological result. That result can be reduction and/or alleviation of a sign, symptom, or cause of a disease or disorder, or any other desired alteration of a biological system. An appropriate effective amount in any individual case may be determined by one of ordinary skill in the art using routine experimentation.

A “therapeutically effective amount” refers to that amount which provides a therapeutic effect for a given condition and administration regimen. In particular, “therapeutically effective amount” means an amount that is effective to prevent, alleviate or ameliorate symptoms of the disease or prolong the survival of the subject being treated, which may be a human or non-human animal. Determination of a therapeutically effective amount is within the skill of the person skilled in the art.

As used herein, the term “stabilizers” refers to either, or both, primary particle and/or secondary stabilizers, which may be polymers or other small molecules. Non-limiting examples of primary particle and/or secondary stabilizers for use with the present invention include, e.g., starch, modified starch, and starch derivatives, gums, including but not limited to polymers, polypeptides, albumin, amino acids, thiols, amines, carboxylic acid and combinations or derivatives thereof. Other examples include xanthan gum, alginic acid, other alginates, benitoniite, veegum, agar, guar, locust bean gum, gum arabic, quince psyllium, flax seed, okra gum, arabinoglactin, pectin, tragacanth, scleroglucan, dextran, amylose, amylopectin, dextrin, etc., cross-linked polyvinylpyrrolidone, ion-exchange resins, potassium polymethacrylate, carrageenan (and derivatives), gum karaya and biosynthetic gum. Other examples of useful primary particle and/or secondary stabilizers include polymers such as: polycarbonates (linear polyesters of carbonic acid); microporous materials (bisphenol, a microporous poly(vinylchloride), micro-porous polyamides, microporous modacrylic copolymers, microporous styrene-acrylic and its copolymers); porous polysulfones, halogenated poly(vinylidene), polychloroethers, acetal polymers, polyesters prepared by esterification of a dicarboxylic acid or anhydride with an alkylene polyol, poly(alkylenesulfides), phenolics, polyesters, asymmetric porous polymers, cross-linked olefin polymers, hydrophilic microporous homopolymers, copolymers or interpolymers having a reduced bulk density, and other similar materials, poly(urethane), cross-linked chain-extended poly(urethane), poly(mides), poly(benzimidazoles), collodion, regenerated proteins, semi-solid cross-linked poly(vinylpyrrolidone).

The terms “coat,” “coated,” or “coating,” as used herein, refer to at least a partial coating of the organic liquid. One hundred percent coverage is not necessarily implied by these terms.

The term “label” when used herein refers to a detectable compound or composition that is conjugated directly or indirectly to a probe to generate a “labeled” probe. The label may be detectable by itself (e.g., radioisotope labels or fluorescent labels) or, in the case of an enzymatic label, may catalyze chemical alteration of a substrate compound or composition that is detectable (e.g., avidin-biotin). In some instances, primers can be labeled to detect a PCR product.

As used herein, the term “specific binding” refers to that binding which occurs between such paired species as enzyme/substrate, receptor/agonist, antibody/antigen, and lectin/carbohydrate which may be mediated by covalent or non-covalent interactions or a combination of covalent and non-covalent interactions. When the interaction of the two species produces a non-covalently bound complex, the binding which occurs is typically electrostatic, hydrogen-bonding, or the result of lipophilic interactions. Accordingly, “specific binding” occurs between a paired species where there is interaction between the two which produces a bound complex having the characteristics of an antibody/antigen or enzyme/substrate interaction. In particular, the specific binding is characterized by the binding of one member of a pair to a particular species and to no other species within the family of compounds to which the corresponding member of the binding member belongs. Thus, for example, an antibody preferably binds to a single epitope and to no other epitope within the family of proteins.

The term “specifically binds”, as used herein with respect to an antibody, is meant for an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

As used herein, the terms “peptide”, “polypeptide”, and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or any combination thereof.

As used herein, “polynucleotide” includes cDNA, RNA, DNA/RNA hybrid, antisense RNA, ribozyme, genomic DNA, synthetic forms, and mixed polymers, both sense and antisense strands, and may be chemically or biochemically modified to contain non-natural or derivatized, synthetic, or semi-synthetic nucleotide bases. Also, contemplated are alterations of a wild type or synthetic gene, including but not limited to deletion, insertion, substitution of one or more nucleotides, or fusion to other polynucleotide sequences.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “nutritional composition” may be a food product intended for human consumption, for example, a beverage, a drink, a bar, a snack, an ice cream, a dairy product, for example a chilled or a shelf-stable dairy product, a fermented dairy product, a drink, for example a milk-based drink, an infant formula, a growing-up milk, a confectionery product, a chocolate, a cereal product such as a breakfast cereal, a sauce, a soup, an instant drink, a frozen product intended for consumption after heating in a microwave or an oven, a ready-to-eat product, a fast food or a nutritional formula.

“Instructional material”, as that term is used herein, includes a publication, a recording, a diagram, or any other medium of expression which can be used to communicate the usefulness of the nucleic acid, peptide, and/or compound of the invention in the kit for identifying, diagnosing or alleviating or treating the various diseases or disorders recited herein. Optionally, or alternately, the instructional material may describe one or more methods of identifying, diagnosing or alleviating the diseases or disorders in a cell or a tissue of a subject. The instructional material of the kit may, for example, be affixed to a container that contains one or more components of the invention or be shipped together with a container that contains the one or more components of the invention. Alternatively, the instructional material may be shipped separately from the container with the intention that the recipient uses the instructional material and the components cooperatively.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Lipid Nanoparticles and Compositions Thereof

In one aspect, the present invention provides a lipid nanoparticle amenable to an irradiation at a wavelength of at or above about 850 nm. In some embodiments, the lipid nanoparticle comprises a dye. In some embodiments, the dye is an aggregate of a dye. In some embodiments, the dye is a J-aggregate of the dye. In some embodiments, the J-aggregate of the dye is encapsulated in the lipid nanoparticle. Thus, in one embodiment, the lipid nanoparticle is a lipid nanocapsule. In one embodiment, the lipid nanoparticle is a lipid nanocarrier. In one embodiment, the lipid nanoparticle is a lipid vesicle. In one embodiment, the lipid nanoparticle is a liposome.

In some embodiments, the lipid nanoparticle comprises a high transition temperature lipid. In some embodiments, the high transition temperature lipid forms the lipid nanoparticle. For example, in some embodiments, the lipid nanoparticle is a lipid nanocapsule comprising a high transition temperature lipid that forms the lipid nanocapsule, and a J-aggregate of a dye that is encapsulated in the lipid nanocapsule. In other embodiments, the lipid nanoparticle is a liposome comprising a high transition temperature lipid that forms the liposome, and a J-aggregate of a dye that is encapsulated in the liposome.

In some embodiments, the high transition temperature lipid has a melting temperature at or above about 50° C. In some embodiments, the high transition temperature lipid has a melting temperature at or above about 56° C. In some embodiments, the high transition temperature lipid has a melting temperature at or above about 60° C. In some embodiments, the high transition temperature lipid has a melting temperature at or above about 65° C. In some embodiments, the high transition temperature lipid has a melting temperature at or above about 70° C. In some embodiments, the high transition temperature lipid has a melting temperature at or above about 75° C. In some embodiments, the high transition temperature lipid has a melting temperature at or above about 78° C. In some embodiments, the high transition temperature lipid has a melting temperature at or above about 80° C.

In some embodiments, the high transition temperature lipid comprises 1,2-dilauroyl-sn-glycero-3-phosphocholine (12:0 PC), 1,2-ditridecanoyl-sn-glycero-3-phosphocholine (13:0 PC), 1,2-dimyristoyl-sn-glycero-3-phosphocholine (14:0 PC), 1,2-dipentadecanoyl-sn-glycero-3-phosphocholine (15:0 PC), 1,2-dipalmitoyl-sn-glycero-3-phosphocholine (16:0 PC), 1,2-diheptadecanoyl-sn-glycero-3-phosphocholine (17:0 PC), 1,2-distearoyl-sn-glycero-3-phosphocholine (18:0 PC or DSPC), 1,2-dinonadecanoyl-sn-glycero-3-phosphocholine (19:0 PC), 1,2-diarachidoyl-sn-glycero-3-phosphocholine (20:0 PC), 1,2-diheneicosanoyl-sn-glycero-3-phosphocholine (21:0 PC), 1,2-dibehenoyl-sn-glycero-3-phosphocholine (22:0 PC), 1,2-ditricosanoyl-sn-glycero-3-phosphocholine (23:0 PC), 1,2-dilignoceroyl-sn-glycero-3-phosphocholine (24:0 PC), 1,2-diheptadecanoyl-sn-glycero-3-phospho-L-serine (17:0 PS), 1,2-distearoyl-sn-glycero-3-phospho-L-serine (18:0 PS), 1,2-dipalmitoyl-sn-glycero-3-phosphate (16:0 PA), 1,2-diheptadecanoyl-sn-glycero-3-phosphate (17:0 PA), 1,2-distearoyl-sn-glycero-3-phosphate (18:0 PA), 1,2-dipalmitoyl-sn-glycero-3-phosphoethanolamine (16:0 PE), 1,2-distearoyl-sn-glycero-3-phosphoethanolamine (18:0 PE), 1,2-diarachidoyl-sn-glycero-3-phosphoethanolamine (20:0 PE), 1,2-distearoyl-sn-glycero-3-phosphoethanolamine-N-diethylenetriaminepentaacetic acid (gadolinium functionalized) (18:0 PE-DTPA (Gd)), or any combination thereof. For example, in some embodiments, the lipid nanoparticle comprises a high transition temperature lipid comprising 18:0 PC, 20:0 PC, 22:0 PC, or any combination thereof. In another embodiment, the lipid nanoparticle comprises 20:0 PC.

In one embodiment, the dye is an aggregate of the dye. In some embodiments, the dye is a J-aggregate of the dye, H-aggregate of the dye, or a combination thereof. For example, in one embodiment, the lipid nanoparticle comprises a J-aggregate of the dye.

In one embodiment, formation of J-aggregates results in narrowing, red-shifting and enhancement of the absorption band. In one embodiment, formation of J-aggregates results in a decreased Stokes shift and enhanced fluorescence.

In some embodiments, the dye has an absorbance in the near infrared (NIR) range between about 650 and 1400 nm. In some embodiments, the dye has an absorbance in the NIR range between about 680 and 1100 nm. In some embodiments, the dye has an absorbance in the NIR range between about 850 and 1100 nm. In some embodiments, the dye has an absorbance in the NIR range between about 700 and 950 nm. In some embodiments, the dye has an absorbance in the NIR range between about 715 and 950 nm. In some embodiments, the dye has an absorbance in the NIR range between about 790 and 895 nm. In some embodiments, the dye has an absorbance in the NIR range between about 885 and 895 nm.

In various embodiments, the dye comprises a polymethine dye. In some embodiments, the polymethine dye comprises a cyanine dye, hemicyanine dye, streptocyanine dye, mercocyanine dye, oxonol dye, styryl dye, diarylmethine dye, triarylmethine dye, rylenes, squaraines, and/or perylene bismides and aza-analogs thereof. Examples of such dyes include, but are not limited to, naphthalocyanine dyes, sulfonated indocyanines, indocyanine green (ICG), Cy3, Cy3.5, Cy5.5, Cy7, pseudoisocyanine chloride, merocyanine I, squarylium dye III, bismide, Congo Red, IR783, Briliant Blue G, rhodamine 6G, U3, F1, thiacarbocyanines, or any combination thereof. For example, in one embodiment, the lipid nanoparticle comprises an indocyanine green (ICG). In another embodiment, the lipid nanoparticle comprises an indocyanine green J-aggregate (ICGJ). In some embodiments, the ICGJ is encapsulated in the lipid nanoparticle. In some embodiments, the ICGJ is encapsulated in the liposome.

In some embodiments, the dye aggregate has a particle size (e.g., average hydrodynamic diameter of the particle) of about 10 nm to about 100 nm. In some embodiments, the dye aggregate has a particle size (e.g., average hydrodynamic diameter of the particle) of about 100 nm to about 150 nm. In some embodiments, the dye aggregate has a particle size (e.g., average hydrodynamic diameter of the particle) of about 200 nm to about 400 nm. In some embodiments, the dye aggregate has a particle size (e.g., average hydrodynamic diameter of the particle) of about 40 nm to about 60 nm.

In some embodiments, the lipid nanoparticle comprises the dye and the high transition temperature in a molar ratio of about 100:1. In some embodiments, the molar ratio of the dye to high transition temperature is about 90:1. In some embodiments, the molar ratio of the dye to high transition temperature is about 80:1. In some embodiments, the molar ratio of the dye to high transition temperature is about 70:1. In some embodiments, the molar ratio of the dye to high transition temperature is about 60:1. In some embodiments, the molar ratio of the dye to high transition temperature is about 50:1. In some embodiments, the molar ratio of the dye to high transition temperature is about 40:1. In some embodiments, the molar ratio of the dye to high transition temperature is about 30:1. In some embodiments, the molar ratio of the dye to high transition temperature is about 20:1. In some embodiments, the molar ratio of the dye to high transition temperature is about 10:1. In some embodiments, the molar ratio of the dye to high transition temperature is about 5:1. In some embodiments, the molar ratio of the dye to high transition temperature is about 2:1. In some embodiments, the molar ratio of the dye to high transition temperature is about 1:1. In some embodiments, the molar ratio of the dye to high transition temperature is about 1:100. In some embodiments, the molar ratio of the dye to high transition temperature is about 1:90. In some embodiments, the molar ratio of the dye to high transition temperature is about 1:80. In some embodiments, the molar ratio of the dye to high transition temperature is about 1:70. In some embodiments, the molar ratio of the dye to high transition temperature is about 1:60. In some embodiments, the molar ratio of the dye to high transition temperature is about 1:50. In some embodiments, the molar ratio of the dye to high transition temperature is about 1:40. In some embodiments, the molar ratio of the dye to high transition temperature is about 1:30. In some embodiments, the molar ratio of the dye to high transition temperature is about 1:20. In some embodiments, the molar ratio of the dye to high transition temperature is about 1:10. In some embodiments, the molar ratio of the dye to high transition temperature is about 1:5. In some embodiments, the molar ratio of the dye to high transition temperature is about 1:2.

In some embodiments, the lipid nanoparticle is amenable to irradiation at a wavelength of between about 800 nm to about 1100 nm. In some embodiments, the lipid nanoparticle is amenable to irradiation at a wavelength of between about 850 nm to about 1100 nm. In some embodiments, the lipid nanoparticle is amenable to irradiation at a wavelength of between about 800 nm to about 890 nm. In some embodiments, the lipid nanoparticle is amenable to irradiation at a wavelength of between about 885 nm to about 895 nm. In some embodiments, the lipid nanoparticle is amenable to irradiation at a wavelength of between about 850 nm to about 900 nm. In some embodiments, the lipid nanoparticle is amenable to irradiation at a wavelength of between about 870 nm to about 910 nm. In some embodiments, the lipid nanoparticle is amenable to irradiation at a wavelength of between about 850 nm to about 950 nm. In some embodiments, the lipid nanoparticle is amenable to irradiation at a wavelength of between about 850 nm to about 1000 nm. In some embodiments, the lipid nanoparticle is amenable to irradiation at a wavelength of between about 900 nm to about 1000 nm. In some embodiments, the lipid nanoparticle is amenable to irradiation at a wavelength of between about 950 nm to about 1000 nm. In some embodiments, the lipid nanoparticle is amenable to irradiation at a wavelength of between about 1000 nm to about 1050 nm. In some embodiments, the lipid nanoparticle is amenable to irradiation at a wavelength of between about 900 nm to about 1100 nm. In some embodiments, the lipid nanoparticle is amenable to irradiation at a wavelength of between about 1050 to 1100 nm.

In some embodiments, the lipid nanoparticle has an absorption wavelength peak from about 800 nm to about 1100 nm. In some embodiments, the lipid nanoparticle has an absorption wavelength peak from about 850 nm to about 1100 nm. In some embodiments, the lipid nanoparticle has an absorption wavelength peak from about 800 nm to about 890 nm. In some embodiments, the lipid nanoparticle has an absorption wavelength peak from about 885 nm to about 895 nm. In some embodiments, the lipid nanoparticle has an absorption wavelength peak from about 850 nm to about 900 nm. In some embodiments, the lipid nanoparticle has an absorption wavelength peak from about 870 nm to about 910 nm. In some embodiments, the lipid nanoparticle has an absorption wavelength peak from about 850 nm to about 950 nm. In some embodiments, the lipid nanoparticle has an absorption wavelength peak from about 850 nm to about 1000 nm. In some embodiments, the lipid nanoparticle has an absorption wavelength peak from about 900 nm to about 1000 nm. In some embodiments, the lipid nanoparticle has an absorption wavelength peak from about 950 nm to about 1000 nm. In some embodiments, the lipid nanoparticle has an absorption wavelength peak from about 1000 nm to about 1050 nm. In some embodiments, the lipid nanoparticle has an absorption wavelength peak from about 900 nm to about 1100 nm. In some embodiments, the lipid nanoparticle has an absorption wavelength peak from about 1050 to 1100 nm.

In various embodiments, the lipid nanoparticle has an average particle size (e.g., average hydrodynamic diameter of the lipid nanoparticle) below about 250 nm. In some embodiments, the lipid nanoparticle has a particle size (i.e., average hydrodynamic diameter of the lipid nanoparticle) of between about 50 nm to about 250 nm. In some embodiments, the lipid nanoparticle has a particle size (i.e., average hydrodynamic diameter of the lipid nanoparticle) of between about 50 nm to about 200 nm. In some embodiments, the lipid nanoparticle has a particle size (i.e., average hydrodynamic diameter of the lipid nanoparticle) of between about 100 nm to about 200 nm. In some embodiments, the lipid nanoparticle has a particle size (i.e., average hydrodynamic diameter of the lipid nanoparticle) of about 100 nm to about 160 nm. For example, in some embodiments, the lipid nanoparticle has a particle size (i.e., average hydrodynamic diameter of the lipid nanoparticle) of about 130 nm±30 nm.

In some embodiments, the lipid nanoparticle is any type of lipid nanoparticle, including, but not limited to, a lipid nanocapsule, lipid nanocluster, lipid nanovesicle, micelle, liposome, dendrimer, and nano-size particle of various other small fabrications that are known to those in the art. In one embodiment, the lipid nanoparticle is a liposome.

In one aspect of the invention, the lipid nanoparticle is a biodegradable lipid nanoparticle. In some embodiments, the lipid nanoparticle is a biodegradable lipid nanocapsule. In some embodiments, the lipid nanoparticle is a biodegradable lipid vesicle. In some embodiments, the lipid nanoparticle is a biodegradable liposome.

In another aspect of the invention, the lipid nanoparticle is a biocompatible lipid nanoparticle. In some embodiments, the lipid nanoparticle is a biocompatible lipid nanocapsule. In some embodiments, the lipid nanoparticle is a biocompatible lipid vesicle. In some embodiments, the lipid nanoparticle is a biocompatible liposome.

In some embodiments, the lipid nanoparticle is resistant to protein opsonization.

In some embodiments, the lipid nanoparticle is a lipid coated nanoparticle.

In some embodiments, the lipid nanoparticle further comprises a polymer. In some embodiments, the polymer is a biocompatible polymer. In some embodiments, the polymer is a biodegradable polymer.

In some embodiments, the polymer is a cationic polymer, anionic polymer, neutral polymer, or any combination thereof.

In some embodiments, the polymer has molecular weight of 5 kDa-3000 kDa. For example, in some embodiments, the polymer has a molecular weight of 5 kDa-2000 kDa, 5 kDa-1500 kDa, 5 kDa-1000 kDa, 5 kDa-800 kDa, 5 kDa-500 kDa, 5 kDa-300 kDa or 5 kDa-200 kDa or 800 kDa-3000 kDa.

The polymer may be a straight chain polymer (i.e., linear polymer) or a branched chain polymer (i.e., branched polymer), including hyperbranched polymers. In some embodiments, the polymer is cross-linked.

In some embodiments, the polymer is a homopolymer. In other embodiments, the polymer a copolymer. In some embodiments, the polymer a block copolymer that is a deblock, triblock, tetrablock, pentablock, or at least six block copolymer.

Examples of such polymers include, but are not limited to, polyethylene glycol (PEG), 1,2-distearoyl-sn-glycero-3-phosphoethanolamine (DSPE), 1,2-distearoyl-sn-glycero-3-phosphoethanolamine-polyethylene glycol (DSPE-PEG), 1,2-distearoyl-sn-glycero-3-phosphoethanolamine-N-[amino(polyethylene glycol)-2000] (DSPE-PEG(2000)-amine), polyethyleneimine (PEI), poly(lactide-co-glycolic acid) (PLGA), biodegradable PLGA, poly(lactide-co-glycolic acid)-polyethylene glycol (PLGA-PEG), biodegradable PLGA-PEG, poly(lactide-co-glycolic acid)-block-polyethylene glycol (PLGA-b-PEG), biodegradable PLGA-b-PEG, poly(ethylene oxide) (PEO), PEO block copolymer, poly(ethylethylene) (PEE), poly(butadiene) (PB or PBD), poly(styrene) (PS), poly(isoprene) (PI), polyanhydride, polyanhydride-block-PEG copolymers, zwitterionic poly(carbobetaine), zwitterionic poly(sulfobetaine)-containing, zwitterionic poly(carbobetaine) and zwitterionic poly(sulfobetaine)-containing copolymers, poly(acrylic acid-co-distearin acrylate), poly(trimethylene carbonate)-block-poly(L-gluatamic acid), poly(ethylene glycol-block-L-aspartic acid), poly(2-hydroxyethyl-co-octadecyl aspartamide), poly(ethylene glycol-co-trimethylene carbonate-co-caprolactone, polypropylene oxide block copolymers, polyethylene oxide-block-polypropylene oxide copolymers, poly(F-caprolactone) (PCL), PCL diblock co-polymer, poly(ethylene oxide)-block-poly(F-caprolactone) (PEO-b-PCL) based diblock copolymers, poly(lactic acid), poly(glycolide), poly(lactic-coglycolic acid), poly(3-hydroxybutyrate), polyamine, polyalkyleneimine (e.g., polyethyleneimine), polyallylamine, polyamidoamine, poly(amino-co-ester), chitosan, poly(2-N,N-dimethylaminoethylmethacrylate), poly-L-lysine, maleimide PEG (mPEG), DSPE-PEG-DBCO, 1-(monomethoxy-polyethyleneglycol)-2,3-dimyristoylglycerol (PEG-s-DMG), DOPE-PEG-Azide, DSPE-PEG-Azide, DPPE-PEG-Azide, DSPE-PEG-Carboxy-NHS, DOPE-PEG-Carboxylic Acid, DSPE-PEG-Carboxylic acid, PEG-modified phosphatidylethanolamine, PEG-modified phosphatidic acid, PEG-modified ceramides (e.g., PEG-CerC14 or PEG-CerC20), PEG-modified dialkylamines, PEG-modified diacylglycerols, PEG-modified dialkylglycerols, PEG-c-DOMG, PEG-c-DMA, PEG-s-DMG, polyethylene glycol-lipid is N-[(methoxy poly(ethylene glycol)₂₀₀₀)carbamyl]-1,2-dimyristyloxlpropyl-3-amine (PEG-c-DMA), polyethylene glycol-lipid is PEG-c-DOMG), pegylated diacylglycerol (PEG-DAG), 1-(monomethoxy-polyethyleneglycol)-2,3-dimyristoylglycerol (PEG-DMG), pegylated phosphatidylethanoloamine (PEG-PE), PEG succinate diacylglycerol (PEG-S-DAG) such as 4-O-(2′,3′-di(tetradecanoyloxy)propyl-1-O-(ω-methoxy(polyethoxy)ethyl)butanedioate (PEG-S-DMG), pegylated ceramide (PEG-cer), PEG dialkoxypropylcarbamate, 1,2-distearoyl-sn-glycero-3-phosphoethanolamine PEG maleimide (DSPE-PEG-maleimide), DSPE-PEG-carboxy-N-hydroxysuccinimide (NHS), DSPE-PEG-folate, DSPE-PEG-biotin, or any combination thereof. For example, in some embodiments, the polymer is PEG.

In some embodiments, the polymer stabilizes the lipid nanoparticle.

In some embodiments, the lipid nanoparticle comprises a high transition temperature lipid and a polymer that form the lipid nanoparticle, and a dye that is encapsulated in the lipid nanoparticle. For example, in some embodiments, the lipid nanoparticle comprises a high transition temperature lipid and PEG that form the lipid nanoparticle, and a J-aggregate of a dye (e.g., ICGJ) that is encapsulated in the lipid nanoparticle.

In some embodiments, the lipid nanoparticle further comprises a second lipid. In some embodiments, the second lipid is a biocompatible lipid. In some embodiments, the second lipid is a biodegradable lipid.

In some embodiments, the lipid nanoparticle comprises a high transition temperature lipid, a polymer, and a second lipid that form the lipid nanoparticle, and a dye that is encapsulated in the lipid nanoparticle. For example, in some embodiments, the lipid nanoparticle comprises a high transition temperature lipid, PEG, and cholesterol that form the lipid nanoparticle, and a J-aggregate of a dye (e.g., ICGJ) that is encapsulated in the lipid nanoparticle.

In some embodiments, the second lipid stabilizes the lipid nanoparticle. In other embodiments, the second lipid is removed from the lipid nanoparticle after the lipid nanoparticle is formed.

Examples of such second lipids include, but are not limited to, cholesterol, N,N-dioleyl-N,N-dimethylammonium chloride (DODAC), N-(2,3-dioleyloxy)propyl)-N,N,N-trimethylammonium chloride (DOTMA), N,N-distearyl-N,N-dimethylammonium bromide (DDAB), N-(2,3-dioleoyloxy)propyl)-N,N,N-trimethylammonium chloride (DOTAP); 3-(N—(N′,N′-dimethylaminoethane)-carbamoyl)cholesterol (DC-Chol), N-(1-(2,3-dioleoyloxy)propyl)-N-2-(sperminecarboxamido)ethyl)-N,N-dimethylammonium trifluoracetate (DOSPA), dioctadecylamidoglycyl carboxyspermine (DOGS), 1,2-dioleoyl-3-dimethylammonium propane (DODAP), N,N-dimethyl-2,3-dioleoyloxy)propylamine (DODMA), and N-(1,2-dimyristyloxyprop-3-yl)-N,N-dimethyl-N-hydroxyethyl ammonium bromide (DMRIE), TRANSFECTAM® (commercially available cationic lipids comprising dioctadecylamidoglycyl carboxyspermine (DOGS) in ethanol from Promega Corp., Madison, Wis.), DODAP, DODMA, DMDMA, 1,2-dilinoleyloxy-N,N-dimethylaminopropane (DLinDMA), 1,2-dilinolenyloxy-N,N-dimethylaminopropane (DLenDMA), 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), 2,2-dilinoleyl-4-dimethylaminomethyl-[1,3]-dioxolane (DLin-K-DMA), PEGylated lipid, such as DSPE-PEG-DBCO, DOPE-PEG-Azide, DSPE-PEG-Azide, DPPE-PEG-Azide, DSPE-PEG-Carboxy-NHS, DOPE-PEG-Carboxylic Acid, and/or DSPE-PEG-Carboxylic acid, diacylphosphatidylcholines, diacylphosphatidylethanolamines, ceramides, sphingomyelins, dihydro sphingomyelins, cephalins, cerebrosides, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), distearoyl-phosphatidylethanolamine (DSPE)-maleimide-PEG, distearoyl-phosphatidylethanolamine (DSPE)-maleimide-PEG2000, 16-O-monomethyl PE, 16-O-dimethyl PE, 18-1-trans PE, 1-stearioyl-2-oleoyl-phosphatidyethanol amine (SOPE), stearoyloleoylphosphatidylcholine (SOPC), 1,2-dielaidoyl-sn-glycero-3-phophoethanolamine (transDOPE), phosphatidylglycerol, cardiolipin, diacylphosphatidylserine, diacylphosphatidic acid, N-dodecanoylphosphatidylethanolamines, N-succinylphosphatidylethanolamines, N-glutarylphosphatidylethanolamines, lysylphosphatidylglycerols, palmitoyloleyolphosphatidylglycerol (POPG), ω-methoxy(polyethoxy)ethyl-N-(2,3-di(tetradecanoxy)propyl)carbamate, 2,3-di(tetradecanoxy)propyl-N-(ω-methoxy(polyethoxy)ethyl)carbamate, or any combination thereof.

In some embodiments, the lipid nanoparticle comprises the high transition temperature lipid, the polymer, and the second lipid in a molar ratio of between about 80 to about 95:between about 30 to about 50:between about 10 to about 15. In some embodiments, the molar ratio of the high transition temperature to polymer to second lipid is between about 80 to about 90:between about 30 to about 50:between about 10 to about 15. In some embodiments, the molar ratio of the high transition temperature to polymer to second lipid is between about 80 to about 90:between about 30 to about 50:between about 10 to about 20.

In some embodiments, the molar ratio of the high transition temperature to polymer to second lipid is about 80:30:10. In some embodiments, the molar ratio of the high transition temperature lipid to the polymer to the second lipid is from about 80:40:10. In some embodiments, the molar ratio of the high transition temperature lipid to the polymer to the second lipid is from about 80:50:10. In some embodiments, the molar ratio of the high transition temperature lipid to the polymer to the second lipid is from about 90:30:10. In some embodiments, the molar ratio of the high transition temperature lipid to the polymer to the second lipid is from about 90:40:10. In some embodiments, the molar ratio of the high transition temperature lipid to the polymer to the second lipid is from about 90:50:10. In one embodiments, the molar ratio of the high transition temperature lipid to the polymer to the second lipid is from about 80:30:15. In some embodiments, the molar ratio of the high transition temperature lipid to the polymer to the second lipid is from about 80:40:15. In some embodiments, the molar ratio of the high transition temperature lipid to the polymer to the second lipid is from about 80:50:15. In some embodiments, the molar ratio of the high transition temperature lipid to the polymer to the second lipid is from about 90:30:15. In some embodiments, the molar ratio of the high transition temperature lipid to the polymer to the second lipid is from about 90:40:15. In some embodiments, the molar ratio of the high transition temperature lipid to the polymer to the second lipid is from about 90:50:15. In one embodiments, the molar ratio of the high transition temperature lipid to the polymer to the second lipid is from about 80:30:20. In some embodiments, the molar ratio of the high transition temperature lipid to the polymer to the second lipid is from about 80:40:20. In some embodiments, the molar ratio of the high transition temperature lipid to the polymer to the second lipid is from about 80:50:20. In some embodiments, the molar ratio of the high transition temperature lipid to the polymer to the second lipid is from about 90:30:20. In some embodiments, the molar ratio of the high transition temperature lipid to the polymer to the second lipid is from about 90:40:20. In some embodiments, the molar ratio of the high transition temperature lipid to the polymer to the second lipid is from about 90:50:20.

In some embodiments, the lipid nanoparticle comprises one or more functional groups. In some embodiments, the functional group is an azide functional group, maleimide functional group, carboxyl functional group, amine functional group, hydrazine functional group, dibenzo-cyclooctyne functional group, or any combination thereof.

In some embodiments, the lipid nanoparticle comprises one or more linker molecules. Examples of linker molecules include, but are not limited to, azide functionalized molecules, maleimide functionalized molecules, carboxyl functionalized molecules, amine functionalized molecules, hydrazine functionalized molecules, dibenzo-cyclooctyne functionalized molecules, or any combination thereof.

In some embodiments, the lipid nanoparticle further comprises a targeting domain. In one aspect, the lipid nanoparticle further comprises a targeting domain attached to the surface of the lipid nanoparticle. In some embodiments, the targeting domain is bound to an exterior surface of the lipid nanoparticle and recognizes a particular site of interest in a subject. In some embodiments, the targeting domain binds to at least one associated with a disease or a disorder. In some embodiments, the targeting domain binds to at least one cancer cell. In some embodiments, the targeting domain binds to at least one cancer antigen. In some embodiments, the targeting domain binds to at least one tumor cell. In some embodiments, the targeting domain binds to at least one cancer biomarker. Examples of cancer biomarkers include, but are not limited to tumor antigens, tumor-specific antigens and tumor-associated antigens, tissue differentiation antigens, mutant protein antigens, epidermal-growth factor receptor (EGFR), oncogenic viral antigens (e.g., alphafetoprotein (AFP) and carcinoembryonic antigen (CEA)), cancer-testis antigens (e.g., CTAG1B and MAGEA1), vascular or stromal specific antigens, epithelial tumor antigen (ETA), tyrosinase, melanoma-associated antigen (MAGE), abnormal products of ras, p53, MUC-1; and tumor markers, such as AFP, carcinoma antigen (CA), CA15-3, CA27-29, CA19-9, CA-125, calcitonin, calretinin, CEA, CD34, CD99MIC 2, CD117, chromogranin, chromosomers 3, 7, 17, and 9p21, cytokeratin (e.g., TPA, TPS, Cyfra21-1, etc.), desmin, epithelial membrane antigen (EMA), factor VIII, CD31, FL1, glial fibrillary acidic protein (GFAP), gross cystic disease fluid protein (GCDFP-15), human melanoma black 45 (HMB-45), human chorionic gonadotropin (hCG), immunoglobulin, inhibin, keratin, Lactate dehydrogenase (LDH), lymphocyte marker, melanoma antigen recognized by T cells 1 (MART-1), Melan-A, myoblast determination protein 1 (Myo D1), muscle-specific actin (MSA), neurofilament, neuron-specific enolase (NSE), placental alkaline phosphatase (PLAP), prostate-specific antigen (PSA), prostatic acid phosphatase (PAP), protein tyrosine phosphatase receptor type C (PTPRC or CD45), S100 protein, smooth muscle actin (SMA), synaptophysin, thymidine kinase, thyroglobulin (Tg), thyroid transcription factor-1 (TTF-1), tumor M2-PK, and vimentin. In some embodiments, the targeting domain binds to at least one epidermal-growth factor receptor (EGFR).

In various embodiments, the targeting domain is an antibody, an antibody fragment, a peptide sequence, aptamer, a ligand, a gene component, or any combination thereof. Examples of targeting domains include, but are not limited to antibodies, lymphokines, cytokines, receptor proteins such as CD4 and CD8, solubilized receptor proteins such as soluble CD4, hormones, growth factors, peptidomimetics, synthetic ligands, and the like which specifically bind desired target cells, and nucleic acids which bind corresponding nucleic acids through base pair complementarity. Targeting domains of particular interest include peptidomimetics, peptides, aptamers, ligands, gene components, antibodies (e.g., monoclonal antibodies, polyclonal antibodies, recombinant antibodies, human antibodies, humanized antibodies, single domain antibodies (nanobodies), etc.) and antibody fragments (e.g., the Fab′ fragment).

In certain embodiments, the targeting domain specifically binds to a tumor-associated antigen (TAA) or tumor specific antigen (TSA). Cellular targets include tissue specific cell surface molecules, for targeting to specific sites of interest (e.g., neural cells, liver cells, bone marrow cells, kidney cells, pancreatic cells, muscle cells, and the like).

Methods of making and using antibodies are well known in the art. For example, polyclonal antibodies useful in the present invention are generated by immunizing rabbits according to standard immunological techniques well-known in the art (see, e.g., Harlow et al., 1988, In: Antibodies, A Laboratory Manual, Cold Spring Harbor, N.Y.). Such techniques include immunizing an animal with a chimeric protein comprising a portion of another protein such as a maltose binding protein or glutathione (GSH) tag polypeptide portion, and/or a moiety such that the antigenic protein of interest is rendered immunogenic (e.g., an antigen of interest conjugated with keyhole limpet hemocyanin, KLH) and a portion comprising the respective antigenic protein amino acid residues.

However, the invention should not be construed as being limited solely to methods and compositions including these antibodies or to these portions of the antigens. Rather, the invention should be construed to include other antibodies, as that term is defined elsewhere herein, to antigens, or portions thereof. Further, the present invention should be construed to encompass antibodies, inter alia, which bind to the specific antigens of interest.

One skilled in the art would appreciate, based upon the disclosure provided herein, that the antibody can specifically bind with any portion of an antigen target, which can be used to generate antibodies specific therefor. However, the present invention is not limited to using the full-length protein as an immunogen. Rather, the present invention includes using an immunogenic portion of the protein to produce an antibody that specifically binds with a specific antigen. That is, the invention includes immunizing an animal using an immunogenic portion, or antigenic determinant, of the antigen.

The antibodies can be produced by immunizing an animal such as, but not limited to, a rabbit, a mouse or a camel, with an antigenic protein of the invention, or a portion thereof, by immunizing an animal using a protein comprising at least a portion of the antigen, or a fusion protein including a tag polypeptide portion comprising, for example, a maltose binding protein tag polypeptide portion, covalently linked with a portion comprising the appropriate amino acid residues. One skilled in the art would appreciate, based upon the disclosure provided herein, that smaller fragments of these proteins can also be used to produce antibodies that specifically bind the antigen of interest.

Once armed with the sequence of a specific antigen of interest and the detailed analysis localizing the various conserved and non-conserved domains of the protein, the skilled artisan would understand, based upon the disclosure provided herein, how to obtain antibodies specific for the various portions of the antigen using methods well-known in the art or to be developed.

Further, the skilled artisan, based upon the disclosure provided herein, would appreciate that using a non-conserved immunogenic portion can produce antibodies specific for the non-conserved region thereby producing antibodies that do not cross-react with other proteins which can share one or more conserved portions. Thus, one skilled in the art would appreciate, based upon the disclosure provided herein, that the non-conserved regions of an antigen of interest can be used to produce antibodies that are specific only for that antigen and do not cross-react non-specifically with other proteins.

The invention encompasses monoclonal, synthetic antibodies, and the like. One skilled in the art would understand, based upon the disclosure provided herein, that the crucial feature of the antibody of the invention is that the antibody bind specifically with an antigen of interest. That is, the antibody of the invention recognizes an antigen of interest or a fragment thereof (e.g., an immunogenic portion or antigenic determinant thereof).

The skilled artisan would appreciate, based upon the disclosure provided herein, that present invention includes use of a single antibody recognizing a single antigenic epitope but that the invention is not limited to use of a single antibody. Instead, the invention encompasses use of at least one antibody where the antibodies can be directed to the same or different antigenic protein epitopes.

The generation of polyclonal antibodies is accomplished by inoculating the desired animal with the antigen and isolating antibodies which specifically bind the antigen therefrom using standard antibody production methods, such as those described in, for example, Harlow et al. (1988, In: Antibodies, A Laboratory Manual, Cold Spring Harbor, N.Y.).

Monoclonal antibodies directed against full length or peptide fragments of a protein or peptide may be prepared using any well-known monoclonal antibody preparation procedures, such as those described, for example, in Harlow et al. (1988, In: Antibodies, A Laboratory Manual, Cold Spring Harbor, N.Y.) and in Tuszynski et al. (1988, Blood, 72:109-115). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

Nucleic acid encoding the monoclonal antibody obtained using the procedures described herein may be cloned and sequenced using technology which is available in the art, and is described, for example, in Wright et al. (1992, Critical Rev. Immunol. 12:125-168), and the references cited therein. Further, the antibody of the invention may be “humanized” using the technology described in, for example, Wright et al., and in the references cited therein, and in Gu et al. (1997, Thrombosis and Hematocyst 77:755-759), and other methods of humanizing antibodies well-known in the art or to be developed.

In some embodiments, a non-human antibody is humanized, where specific sequences or regions of the antibody are modified to increase similarity to an antibody naturally produced in a human or fragment thereof. A humanized antibody can be produced using a variety of techniques known in the art, including but not limited to, CDR-grafting (see, e.g., European Patent No. EP 239,400; International Publication No. WO 91/09967; and U.S. Pat. Nos. 5,225,539, 5,530,101, and 5,585,089, each of which is incorporated herein in its entirety by reference), veneering or resurfacing (see, e.g., European Patent Nos. EP 592,106 and EP 519,596; Padlan, 1991, Molecular Immunology, 28(4/5):489-498; Studnicka et al., 1994, Protein Engineering, 7(6):805-814; and Roguska et al., 1994, PNAS, 91:969-973, each of which is incorporated herein by its entirety by reference), chain shuffling (see, e.g., U.S. Pat. No. 5,565,332, which is incorporated herein in its entirety by reference), and techniques disclosed in, e.g., U.S. Patent Application Publication No. US2005/0042664, U.S. Patent Application Publication No. US2005/0048617, U.S. Pat. Nos. 6,407,213, 5,766,886, International Publication No. WO 9317105, Tan et al., J. Immunol., 169:1119-25 (2002), Caldas et al., Protein Eng., 13(5):353-60 (2000), Morea et al., Methods, 20(3):267-79 (2000), Baca et al., J. Biol. Chem., 272(16):10678-84 (1997), Roguska et al., Protein Eng., 9(10):895-904 (1996), Couto et al., Cancer Res., 55 (23 Supp):5973s-5977s (1995), Couto et al., Cancer Res., 55(8):1717-22 (1995), Sandhu J S, Gene, 150(2):409-10 (1994), and Pedersen et al., J. Mol. Biol., 235(3):959-73 (1994), each of which is incorporated herein in its entirety by reference. Often, framework residues in the framework regions will be substituted with the corresponding residue from the CDR donor antibody to alter, for example improve, antigen binding. These framework substitutions are identified by methods well-known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature, 332:323, which are incorporated herein by reference in their entireties.)

In some embodiments, the antibody fragment provided herein is a single chain variable fragment (scFv). In various embodiments, the antibodies of the invention may exist in a variety of other forms including, for example, Fv, Fab, and (Fab′) 2, as well as bi-functional (i.e. bi-specific) hybrid antibodies (e.g., Lanzavecchia et al., Eur. J. Immunol. 17, 105 (1987)). In some embodiments, the antibodies and fragments thereof of the invention bind a cell bearing antigen, TCR, and/or BCR with wild-type or enhanced affinity. In some embodiments, the antibodies and fragments thereof of the invention bind a T cell bearing TCR with wild-type or enhanced affinity. In some embodiments, the antibodies and fragments thereof of the invention bind a B cell bearing BCR with wild-type or enhanced affinity. In various embodiments, a human scFv may also be derived from a yeast display library.

ScFvs can be prepared according to method known in the art (see, for example, Bird et al., (1988) Science 242:423-426 and Huston et al., (1988) Proc. Natl. Acad. Sci. USA 85:5879-5883). ScFv molecules can be produced by linking VH and VL regions together using flexible polypeptide linkers. The scFv molecules comprise flexible polypeptide linker (e.g., a Ser-Gly linker) with an optimized length and/or amino acid composition. The flexible polypeptide linker length can greatly affect how the variable regions of an scFv fold and interact. In fact, if a short polypeptide linker is employed (e.g., between 5-10 amino acids, intrachain folding is prevented. Interchain folding is also required to bring the two variable regions together to form a functional epitope binding site. For examples of linker orientation and size see, e.g., Hollinger et al. 1993 Proc Natl Acad. Sci. U.S.A. 90:6444-6448, U.S. Patent Application Publication Nos. 2005/0100543, 2005/0175606, 2007/0014794, and PCT publication Nos. WO2006/020258 and WO2007/024715, is incorporated herein by reference.

The scFv can comprise a polypeptide linker sequence of at least 1, 2, 3, 4, 5, 6, 7, 8, 9, 10, 11, 12, 13, 14, 15, 16, 17, 18, 19, 20, 25, 30, 35, 40, 45, 50, or more amino acid residues between its VL and VH regions. The flexible polypeptide linker sequence may comprise any naturally occurring amino acid. In some embodiments, the flexible polypeptide linker sequence comprises amino acids glycine and serine. In another embodiment, the flexible polypeptide linker sequence comprises sets of glycine and serine repeats such as (Gly4Ser)n, where n is a positive integer equal to or greater than 1. In some embodiments, the flexible polypeptide linkers include, but are not limited to, (Gly4Ser)4 or (Gly4Ser)3. Variation in the flexible polypeptide linker length may retain or enhance activity, giving rise to superior efficacy in activity studies.

In some embodiments, the targeting domain is bound directly to the lipid nanoparticle. In some embodiments, the targeting domain is bound directly to the surface of the lipid nanoparticle. In some embodiments, the targeting domain is bound directly to the cationic polymer. In some embodiments, the targeting domain is bound to the lipid nanoparticle using a linking molecule. In some embodiments, the targeting domain is bound to the surface of the lipid nanoparticle using a linking molecule. In some embodiments, the targeting domain is bound to the cationic polymer using a linking molecule.

The linking molecules useful in the compositions and methods of the present disclosure may be any molecule capable of binding to both the coating material used in the compositions and methods of the present disclosure and the targeting domains used in the compositions and methods of the present disclosure. In certain embodiments, the linking molecule may be a hydrophilic polymer. Examples of linking molecules include, but are not limited to, poly(ethylene glycol) and its derivatives, azide compounds, maleimide compounds, hydzrazine compounds, dibenzo-cyclooctyne (DBCO) compounds, dithiol compounds, dithiol compounds with hydrazide and/or carboxylic functionality, or single thiols and/or amines or their derivatives.

In certain embodiments, the linking molecule and the targeting domain may be bound by one or more covalent bonds. In certain embodiments, the linking molecule, in addition to linking the targeting domain and the coating material, may impart certain benefits upon the compositions of the present disclosure, including, but not limited to, improved hydrophilicity and stability in solution, reduced immunogenic responses upon introduction of the compositions of the present disclosure into a subject, increased circulation time of the compositions of the present disclosure when introduced into the bloodstream of a subject. The choice of a linking molecule may depend upon, among other things, the targeting domain chosen and the subject into which the compositions of the present invention are to be introduced. One of ordinary skill in the art, with the benefit of this disclosure, will recognize additional suitable linking molecules. Such linking molecules are considered to be within the spirit of the present disclosure.

In certain embodiments, the targeting domain may recognize a particular ligand or receptor present in a desired cell and/or tissue type when introduced into a subject. In certain embodiments, the targeting domain may be an antibody that recognizes such a particular ligand or receptor. The use of antibody fragments may also be suitable in the compositions of the present disclosure. The choice of a targeting domain may depend upon, among other things, the cell and/or tissue type into which an at least partial increase in uptake of the compositions of the present disclosure is desired, as well as particular ligand(s) present in such cell and/or tissue types.

In certain embodiments, the targeting domain may be chosen, among other things, to at least partially increase the uptake of the compositions of the present disclosure into a desired cell and/or tissue type when introduced into a subject. In certain embodiments, the targeting domain may be a moiety that recognizes a molecule which is present in higher amounts in an abnormal form of a tissue when compared to a normal form of the same tissue (i.e., the molecule is “up-regulated” in the abnormal form of the tissue).

In some embodiments, the suitable targeting domain may be a peptide sequence, DNA fragment, aptamer, RNA, polymer, etc. One of ordinary skill in the art, with the benefit of this disclosure, will recognize other targeting domains that may be useful in the compositions of the present disclosure. Such targeting domains are considered to be within the spirit of the present disclosure.

In one aspect, the lipid nanoparticle comprises one or more antibodies. In some embodiments, the lipid nanoparticle is bound to the antibody. In some embodiments, the high transition temperature lipid is conjugated with at least one antibody. In some embodiments, the encapsulated dye (e.g., ICGJ) is conjugated with at least one antibody. In some embodiments, the polymer (e.g., PEG) is conjugated with at least one antibody. In some embodiments, the second lipid (e.g., cholesterol) is conjugated with at least one antibody.

In some embodiments, the antibody selectively binds cells associated with a disease or a disorder. In some embodiments, the antibody selectively binds cancer cells. In some embodiments, the antibody selectively binds tumor cells. In some embodiments, the antibody is specific for EGFR. In various embodiments, the antibody is a monoclonal antibody (mAb). In some embodiments, the mAb is a grafted mAb.

In one aspect of the invention, the lipid nanoparticle further comprises one or more therapeutic agents. In some embodiments, the lipid nanoparticle is bound to the therapeutic agent. In one embodiment, the therapeutic agent is attached to the surface of the lipid nanoparticle. In some embodiments, the lipid nanoparticle encapsulates at least one dye and at least one therapeutic agent.

In one embodiment, the therapeutic agent is a chemotherapeutic agent.

In some embodiments, the therapeutic agent is a hydrophobic therapeutic agent. In some embodiments, the therapeutic agent is a hydrophilic therapeutic agent. Examples of such therapeutic agents include, but are not limited to, one or more drugs, proteins, amino acids, peptides, antibodies, antibiotics, small molecules, anti-cancer agents, chemotherapeutic agents, immunomodulatory agents, RNA molecules, siRNA molecules, DNA molecules, gene editing agents, gene-silencing agents, CRISPR-associated agents (e.g., guide RNA molecules, endonucleases, and variants thereof), medical imaging agents, therapeutic moieties, one or more non-therapeutic moieties or a combination to target cancer or atherosclerosis, selected from folic acid, peptides, proteins, aptamers, antibodies, siRNA, poorly water soluble drugs, anti-cancer drugs, antibiotics, analgesics, vaccines, anticonvulsants; anti-diabetic agents, antifungal agents, antineoplastic agents, anti-parkinsonian agents, anti-rheumatic agents, appetite suppressants, biological response modifiers, cardiovascular agents, central nervous system stimulants, contraceptive agents, dietary supplements, vitamins, minerals, lipids, saccharides, metals, amino acids (and precursors), nucleic acids and precursors, contrast agents, diagnostic agents, dopamine receptor agonists, erectile dysfunction agents, fertility agents, gastrointestinal agents, hormones, immunomodulators, antihypercalcemia agents, mast cell stabilizers, muscle relaxants, nutritional agents, ophthalmic agents, osteoporosis agents, psychotherapeutic agents, parasympathomimetic agents, parasympatholytic agents, respiratory agents, sedative hypnotic agents, skin and mucous membrane agents, smoking cessation agents, steroids, sympatholytic agents, urinary tract agents, uterine relaxants, vaginal agents, vasodilator, anti-hypertensive, hyperthyroids, anti-hyperthyroids, anti-asthmatics and vertigo agents, or any combinations thereof.

In some embodiments, the therapeutic agent is one or more non-therapeutic moieties. In some embodiments, the lipid nanoparticle comprises one or more therapeutic moieties, one or more non-therapeutic moieties, or any combination thereof. In some embodiments, the therapeutic moiety targets cancer. In some embodiments, the composition comprises folic acid, peptides, proteins, aptamers, antibodies, small RNA molecules, miRNA, shRNA, siRNA, poorly water-soluble therapeutic agents, anti-cancer agents, or any combinations thereof.

In some embodiments, the therapeutic agent may be an anti-cancer agent. Any suitable anti-cancer agent may be used in the compositions and methods of the present disclosure. The selection of a suitable anti-cancer agent may depend upon, among other things, the type of cancer to be treated and the lipid nanoparticle compositions of the present disclosure. In certain embodiments, the anti-cancer agent may be effective for treating one or more of pancreatic cancer, esophageal cancer, rectal cancer, colon cancer, prostate cancer, kidney cancer, liver cancer, breast cancer, ovarian cancer, and stomach cancer. Examples of anti-cancer agents include, but is not limited to, chemotherapeutic agents, antiproliferative agents, anti-tumor agents, checkpoint inhibitors, and anti-angiogenic agents. For example, in some embodiments, the anti-cancer agent is gemcitabine, doxorubicin, 5-Fu, tyrosine kinase inhibitors, sorafenib, trametinib, rapamycin, fulvestrant, ezalutamide, or paclitaxel.

Chemotherapeutic agents include cytotoxic agents (e.g., 5-fluorouracil, cisplatin, carboplatin, methotrexate, daunorubicin, doxorubicin, vincristine, vinblastine, oxorubicin, carmustine (BCNU), lomustine (CCNU), cytarabine USP, cyclophosphamide, estramucine phosphate sodium, altretamine, hydroxyurea, ifosfamide, procarbazine, mitomycin, busulfan, cyclophosphamide, mitoxantrone, carboplatin, cisplatin, interferon alfa-2a recombinant, paclitaxel, teniposide, and streptozoci), cytotoxic alkylating agents (e.g., busulfan, chlorambucil, cyclophosphamide, melphalan, or ethylesulfonic acid), alkylating agents (e.g., asaley, AZQ, BCNU, busulfan, bisulphan, carboxyphthalatoplatinum, CBDCA, CCNU, CHIP, chlorambucil, chlorozotocin, cis-platinum, clomesone, cyanomorpholinodoxorubicin, cyclodisone, cyclophosphamide, dianhydrogalactitol, fluorodopan, hepsulfam, hycanthone, iphosphamide, melphalan, methyl CCNU, mitomycin C, mitozolamide, nitrogen mustard, PCNU, piperazine, piperazinedione, pipobroman, porfiromycin, spirohydantoin mustard, streptozotocin, teroxirone, tetraplatin, thiotepa, triethylenemelamine, uracil nitrogen mustard, and Yoshi-864), antimitotic agents (e.g., allocolchicine, Halichondrin M, colchicine, colchicine derivatives, dolastatin 10, maytansine, rhizoxin, paclitaxel derivatives, paclitaxel, thiocolchicine, trityl cysteine, vinblastine sulfate, and vincristine sulfate), plant alkaloids (e.g., actinomycin D, bleomycin, L-asparaginase, idarubicin, vinblastine sulfate, vincristine sulfate, mitramycin, mitomycin, daunorubicin, VP-16-213, VM-26, navelbine and taxotere), biologicals (e.g., alpha interferon, BCG, G-CSF, GM-CSF, and interleukin-2), topoisomerase I inhibitors (e.g., camptothecin, camptothecin derivatives, and morpholinodoxorubicin), topoisomerase II inhibitors (e.g., mitoxantron, amonafide, m-AMSA, anthrapyrazole derivatives, pyrazoloacridine, bisantrene HCL, daunorubicin, deoxydoxorubicin, menogaril, N,N-dibenzyl daunomycin, oxanthrazole, rubidazone, VM-26 and VP-16), and synthetics (e.g., hydroxyurea, procarbazine, o,p′-DDD, dacarbazine, CCNU, BCNU, cis-diamminedichloroplatimun, mitoxantrone, CBDCA, levamisole, hexamethylmelamine, all-trans retinoic acid, gliadel and porfimer sodium).

Antiproliferative agents are compounds that decrease the proliferation of cells. Antiproliferative agents include alkylating agents, antimetabolites, enzymes, biological response modifiers, miscellaneous agents, hormones and antagonists, androgen inhibitors (e.g., flutamide and leuprolide acetate), antiestrogens (e.g., tamoxifen citrate and analogs thereof, toremifene, droloxifene and roloxifene), Additional examples of specific antiproliferative agents include, but are not limited to levamisole, gallium nitrate, granisetron, sargramostim strontium-89 chloride, filgrastim, pilocarpine, dexrazoxane, and ondansetron.

The inhibitors of the invention can be administered alone or in combination with other anti-tumor agents, including cytotoxic/antineoplastic agents and anti-angiogenic agents. Cytotoxic/anti-neoplastic agents are defined as agents which attack and kill cancer cells. Some cytotoxic/anti-neoplastic agents are alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents are antimetabolites for tumor cells, e.g., cytosine arabinoside, fluorouracil, methotrexate, mercaptopuirine, azathioprime, and procarbazine. Other cytotoxic/anti-neoplastic agents are antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents are mitotic inhibitors (vinca alkaloids). These include vincristine, vinblastine and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine.

Anti-angiogenic agents are well known to those of skill in the art. Suitable anti-angiogenic agents for use in the methods and compositions of the present disclosure include anti-VEGF antibodies, including humanized and chimeric antibodies, anti-VEGF aptamers and antisense oligonucleotides. Other known inhibitors of angiogenesis include angiostatin, endostatin, interferons, interleukin 1 (including alpha and beta) interleukin 12, retinoic acid, and tissue inhibitors of metalloproteinase-1 and -2. (TIMP-1 and -2). Small molecules, including topoisomerases such as razoxane, a topoisomerase II inhibitor with anti-angiogenic activity, can also be used.

Other anti-cancer agents that can be used in combination with the disclosed compounds include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. In some embodiments, the anti-cancer drug is 5-fluorouracil, taxol, or leucovorin.

In some embodiments, the anti-cancer agent may be a prodrug form of an anti-cancer agent. As used herein, the term “prodrug form” and its derivatives is used to refer to a drug that has been chemically modified to add and/or remove one or more substituents in such a manner that, upon introduction of the prodrug form into a subject, such a modification may be reversed by naturally occurring processes, thus reproducing the drug. The use of a prodrug form of an anti-cancer agent in the compositions, among other things, may increase the concentration of the anti-cancer agent in the compositions of the present disclosure. In certain embodiments, an anti-cancer agent may be chemically modified with an alkyl or acyl group or some form of lipid.

The selection of such a chemical modification, including the substituent(s) to add and/or remove to create the prodrug, may depend upon a number of factors including, but not limited to, the particular drug and the desired properties of the prodrug. One of ordinary skill in the art, with the benefit of this disclosure, will recognize suitable chemical modifications.

The present invention also provides various compositions comprising at least one lipid nanoparticle of the present invention. In some embodiments, the composition comprises a lipid nanoparticle and a dye. In some embodiments, the composition comprises the dye encapsulated in the lipid nanoparticle.

In some embodiments, the composition is a biodegradable composition. In some embodiments, the composition is a medical biodegradable composition.

In some embodiments, the composition is a biocompatible composition. In some embodiments, the composition is a medical biocompatible composition.

In some embodiments, the composition is a pharmaceutical composition.

In some embodiments, the composition further comprises at least one pharmaceutically acceptable carrier.

In various aspects, the composition comprises: one or more lipid nanoparticles of the present invention and one or more stabilizers. In various embodiments, the stabilizer to nanoparticle weight ratio is less than 50%. In some embodiments, the stabilizer comprises a biocompatible polymer. Examples of stabilizers include, but are not limited to, biocompatible polymer, a biodegradable polymer, a multifunctional linker, starch, modified starch, and starch derivatives, gums, including but not limited to polymers, polypeptides, albumin, amino acids, alcohols (e.g., PVA, ethyl alcohol, etc.), thiols, amines, carboxylic acid and combinations or derivatives thereof, citric acid, xanthan gum, alginic acid, other alginates, benitoniite, veegum, agar, guar, locust bean gum, gum arabic, quince psyllium, flax seed, okra gum, arabinoglactin, pectin, tragacanth, scleroglucan, dextran, amylose, amylopectin, dextrin, etc., cross-linked polyvinylpyrrolidone, ion-exchange resins, potassium polymethacrylate, carrageenan (and derivatives), gum karaya and biosynthetic gum, polycarbonates (linear polyesters of carbonic acid); microporous materials (bisphenol, a microporous poly(vinylchloride), micro-porous polyamides, microporous modacrylic copolymers, microporous styrene-acrylic and its copolymers); porous polysulfones, halogenated poly(vinylidene), polychloroethers, acetal polymers, polyesters prepared by esterification of a dicarboxylic acid or anhydride with an alkylene polyol, poly(alkylenesulfides), phenolics, polyesters, asymmetric porous polymers, cross-linked olefin polymers, hydrophilic microporous homopolymers, copolymers or interpolymers having a reduced bulk density, and other similar materials, poly(urethane), cross-linked chain-extended poly(urethane), poly(imides), poly(benzimidazoles), collodion, regenerated proteins, semi-solid cross-linked poly(vinylpyrrolidone), monomeric, dimeric, oligomeric or long-chain, copolymers, block polymers, block co-polymers, polymers, PEG, dextran, modified dextran, polyvinylalcohol, polyvinylpyrollidone, polyacrylates, polymethacrylates, polyanhydrides, polypeptides, albumin, alginates, amino acids, thiols, amines and carboxylic acids or combinations thereof.

In various aspects, the lipid nanoparticle of the present invention may be delivered to a cell or biological tissue of interest by a red-blood cell-hitchhiking methods that are well-known to those of skill in the art, and such methods are incorporated herein by reference. Examples of such red-blood cell-hitchhiking methods are described in Brenner et al., 2018, Nature Commun., 9:2684.

In various embodiments, the clearance, excretion rates, and pathways of the lipid nanoparticles can be predetermined by the size (e.g., average diameter) of the lipid nanoparticles and physiochemical properties of capping ligands and templating polymers. This approach provides a flexible platform for designing and validation of various types of lipid nanoparticles for safe clinical use. For example, different types of lipid nanomaterials can be clustered together providing multiplexing opportunities for synthesis of multifunctional/multimodal nanoparticles.

In some embodiments, the composition is an imaging agent.

In some embodiments, the composition is a contrast agent.

Imaging and/or Contrast Agents

The present invention also provides an imaging and/or contrast agent comprising at least one lipid nanoparticle of the present invention. In various aspects, the imaging and/or contrast agent is a photoacoustic imaging (PAI) contrast agent, ultrasound imaging contrast agent, optical imaging contrast agent, magnetic resonance imaging contrast agent, computed tomography contrast agent, thermal imaging contrast agent, nuclear imaging contrast agent, magnetomotive imaging enhancement contrast agent, fluorescence imaging contrast agent, and any combination thereof.

In various aspects, the composition of the present invention acts as an imaging and/or contrast agent for continuous wave photoacoustic imaging, combined photoacoustic and ultrasound imaging, magnetomotive imaging, optical coherent tomography, magnetic resonance imaging, computed tomography, nuclear imaging modalities, or any combination thereof. Furthermore, when the composition contains magnetic iron oxide and/or cobalt nanoparticles, it may be used in microwave ablation therapy and magnetomotive imaging enhancement.

In some embodiments, the lipid nanoparticle is used as contrast enhancement for optical imaging methods, such as optical coherence tomography, magnetic resonance imaging, computed tomography, fluorescence imaging, and photoacoustic imaging (for example, through mechanisms of vaporization and thermal expansion).

In various embodiments, the imaging and/or contrast agents comprise self-assembled aggregates comprising the lipid nanoparticles of the present invention. In some embodiments, the aggregates are cross-linked.

In some embodiments, biologically active agents may be added to the lipid nanoparticles for molecular specific optoacoustic imaging of cancer cells. In some embodiments, the composition of the present invention is combined with a contrast medium. Exemplary contrast mediums include, but are not limited to, polyethylene glycol (PEG), gadolinium, iodine, PEGylated gadolinium, and combinations thereof.

In one aspect, the imaging and/or contrast agent comprises the lipid nanoparticle of the present invention and a pharmaceutically acceptable excipient. In various embodiments, the imaging and/or contrast agent further comprises an additional organic component. In some embodiments, the organic component is a lipid composition. In some embodiments, the organic component comprises one or more targeting domains. In some embodiments, the lipid nanoparticle is complexed with a lipid. In some embodiments, the lipid comprises at least 50% diacetylene phospholipid. In some embodiments, the lipid further comprises at least one targeting lipid. In some embodiments, the targeting lipid comprises a targeting domain specific for a cancer cell. In some embodiments, the lipid nanoparticles of the present invention are bound to the surface of the organic component. Examples of such organic surface include, but are not limited to, a protein surface, lipid surface, or a combination thereof.

To obtain selectivity, the imaging and/or contrast agent may be passively or actively targeted to regions of diagnostic interest, such as organs, vessels, sites of disease, tumorous tissue, or a specific organism in a subject. In active targeting, the imaging and/or contrast agents may be attached to biological recognition agents to allow them to accumulate in or to be selectively retained by or to be slowly eliminated from certain parts of the body, such as specific organs, parts of organs, bodily structures and disease structures and lesions. Active targeting is defined as a modification of biodistribution using chemical groups that will associate with species present in the desired tissue or organism to effectively decrease the rate of loss of contrast agent from the specific tissue or organism.

Active targeting of an imaging and/or contrast agent can be considered as localization through modification of biodistribution of the imaging and/or contrast agent by means of a targeting domain that is attached to or incorporated into the imaging and/or contrast agent. The targeting domain can associate or bind with one or more receptor species present in the tissue or organism of diagnostic interest. This binding will effectively decrease the rate of loss of contrast agent from the specific tissue or organism of diagnostic interest. In such cases, the imaging and/or contrast agent can be modified synthetically to incorporate the targeting domain. Targeted contrast agents can localize because of binding between the ligand and the targeted receptor. Alternatively, contrast agents can distribute by passive biodistribution, i.e., by passive targeting, into diseased tissues of interest such as tumors. Thus, even without synthetic manipulation to incorporate a targeting domain that can bind to a receptor site, passively targeted contrast agents can accumulate in a diseased tissue or in specific locations in the subject, such as the liver. The present invention comprises use of an imaging and/or contrast agent that is linked to a targeting domain that has an affinity for binding to a receptor. Preferably the receptor is located on the surface of a diseased or disease-causing cell in a human or animal subject.

The imaging and/or contrast agents are formulated in a pharmaceutically acceptable excipient, such as wetting agents, buffers, disintegrants, binders, fillers, flavoring agents and liquid carrier media such as sterile water, water/ethanol etc. The imaging and/or contrast agent should be suitable for administration either by injection or inhalation or catheterization or instillation or transdermal introduction into any of the various body cavities including the alimentary canal, the vagina, the rectum, the bladder, the ureter, the urethra, the mouth, etc. For oral administration, the pH of the composition is preferably in the acid range (e.g., 2 to 7) and buffers or pH adjusting agents may be used. The contrast media may be formulated in conventional pharmaceutical administration forms, such as tablets, capsules, powders, solutions, dispersion, syrups, suppositories etc.

### Method of Preparation

The present invention relates, in part, to a novel method of generating lipid nanoparticles described herein. The present invention relates, in part, to a novel method of generating compositions described herein comprising the steps of: a) generating a lipid nanoparticle; b) adding a dye; c) encapsulating the dye in the lipid nanoparticle; and d) heating the dye encapsulated in the lipid nanoparticle to generate a J-aggregate of the dye encapsulated by the lipid nanoparticle. In some embodiments, the lipid nanoparticle comprises a high transition temperature lipid having a melting temperature of above about 60° C.

In some embodiments, the step of generating a lipid nanoparticle comprises mixing of a high transition temperature lipid.

In various embodiments, the high transition temperature lipid is any high transition temperature lipid described herein. For example, in some embodiments, the high transition temperature lipid has a melting temperature of about 56° C. In another embodiment, the high transition temperature lipid has a melting temperature of about 60° C. In some embodiments, the high transition temperature lipid comprises 1,2-distearoyl-sn-glycero-3-phosphocholine (18:0 PC or DSPC), 1,2-diarachidoyl-sn-glycero-3-phosphocholine (20:0 PC), 1,2-dibehenoyl-sn-glycero-3-phosphocholine (22:0 PC), or any combination thereof.

In some embodiments, the step of generating a lipid nanoparticle comprises mixing a high transition temperature lipid with a polymer and a second lipid.

In various embodiments, the polymer is any polymer described herein. For example, in some embodiments, the polymer is polyethylene glycol (PEG), 1,2-distearoyl-sn-glycero-3-phosphoethanolamine-polyethylene glycol (DSPE-PEG), 1,2-distearoyl-sn-glycero-3-phosphoethanolamine-N-[amino(polyethylene glycol)-2000] (DSPE-PEG(2000)-amine), or any combination thereof.

In various embodiments, the second lipid is any lipid described herein. For example, in some embodiments, the second lipid is cholesterol.

In some embodiments, the method comprises a step of removing the second lipid after the step of generating a lipid nanoparticle. In some embodiments, the step of removing the second lipid comprises evaporating the second lipid. For example, in some embodiments, the step of removing the second lipid comprises evaporating the second lipid under reduced pressure.

In some embodiments, the step of removing the second lipid comprises evaporating the second lipid under an inert gas (e.g., argon, nitrogen, etc.).

In some embodiments, the step of generating a lipid nanoparticle comprises mixing a high transition temperature lipid with a polymer and a second lipid at the molar ratio of between about 80 to about 95 of the high transition temperature lipid to between about 30 to about 50 of the polymer to between about 10 to about 15 of the second lipid. In some embodiments, the step of generating a lipid nanoparticle comprises mixing a high transition temperature lipid with a polymer and a second lipid at the molar ratio of between about 80 to about 90 of the high transition temperature lipid to between about 30 to about 50 of the polymer to between about 10 to about 15 of the second lipid.

For example, in some embodiments, the step of generating a lipid nanoparticle comprises mixing a high transition temperature lipid with a polymer and a second lipid at the molar ratio of about 80:about 30:about 10 of the high transition temperature lipid to the polymer to the second lipid. In some embodiments, the molar ratio of the high transition temperature lipid to the polymer to the second lipid is from about 80:40:10. In some embodiments, the molar ratio of the high transition temperature lipid to the polymer to the second lipid is from about 80:50:10. In some embodiments, the molar ratio of the high transition temperature lipid to the polymer to the second lipid is from about 90:30:10. In some embodiments, the molar ratio of the high transition temperature lipid to the polymer to the second lipid is from about 90:40:10. In some embodiments, the molar ratio of the high transition temperature lipid to the polymer to the second lipid is from about 90:50:10. In one embodiments, the molar ratio of the high transition temperature lipid to the polymer to the second lipid is from about 80:30:15. In some embodiments, the molar ratio of the high transition temperature lipid to the polymer to the second lipid is from about 80:40:15. In some embodiments, the molar ratio of the high transition temperature lipid to the polymer to the second lipid is from about 80:50:15. In some embodiments, the molar ratio of the high transition temperature lipid to the polymer to the second lipid is from about 90:30:15. In some embodiments, the molar ratio of the high transition temperature lipid to the polymer to the second lipid is from about 90:40:15. In some embodiments, the molar ratio of the high transition temperature lipid to the polymer to the second lipid is from about 90:50:15.

In some embodiments, the step of adding a dye comprises mixing the dye and lipid nanoparticle at a temperature of between about 60° C. to about 85° C. In some embodiments, the step of adding a dye comprises mixing the dye and lipid nanoparticle at a temperature of between about 60° C. to about 80° C. In some embodiments, the step of adding a dye comprises mixing the dye and lipid nanoparticle at a temperature of between about 65° C. to about 85° C. In some embodiments, the step of adding a dye comprises mixing the dye and lipid nanoparticle at a temperature of between about 70° C. to about 80° C. In some embodiments, the step of adding a dye comprises mixing the dye and lipid nanoparticle at a temperature of between about 70° C. to about 85° C. For example, in one embodiment, the step of adding a dye comprises mixing the dye and lipid nanoparticle at a temperature of about 80° C.

In some embodiments, the step of heating the dye encapsulated in the lipid nanoparticle to generate a J-aggregate of the dye encapsulated by the lipid nanoparticle comprises heating the dye encapsulated in the lipid nanoparticle at a temperature of about 60° C. for between about 14 hr to about 30 hr. In some embodiments, the dye encapsulated in the lipid nanoparticle is heated at a temperature of about 60° C. for between about 15 hr to about 30 hr. In some embodiments, the dye encapsulated in the lipid nanoparticle is heated at a temperature of about 60° C. for between about 16 hr to about 30 hr. In some embodiments, the dye encapsulated in the lipid nanoparticle is heated at a temperature of about 60° C. for between about 17 hr to about 30 hr. In some embodiments, the dye encapsulated in the lipid nanoparticle is heated at a temperature of about 60° C. for between about 18 hr to about 30 hr. In some embodiments, the dye encapsulated in the lipid nanoparticle is heated at a temperature of about 60° C. for between about 19 hr to about 30 hr. In some embodiments, the dye encapsulated in the lipid nanoparticle is heated at a temperature of about 60° C. for between about 20 hr to about 30 hr. In some embodiments, the dye encapsulated in the lipid nanoparticle is heated at a temperature of about 60° C. for between about 21 hr to about 30 hr. In some embodiments, the dye encapsulated in the lipid nanoparticle is heated at a temperature of about 60° C. for between about 22 hr to about 30 hr. In some embodiments, the dye encapsulated in the lipid nanoparticle is heated at a temperature of about 60° C. for between about 23 hr to about 30 hr. In some embodiments, the dye encapsulated in the lipid nanoparticle is heated at a temperature of about 65° C. for between about 14 hr to about 30 hr. In some embodiments, the dye encapsulated in the lipid nanoparticle is heated at a temperature of about 65° C. for between about 15 hr to about 30 hr. In some embodiments, the dye encapsulated in the lipid nanoparticle is heated at a temperature of about 65° C. for between about 16 hr to about 30 hr. In some embodiments, the dye encapsulated in the lipid nanoparticle is heated at a temperature of about 65° C. for between about 17 hr to about 30 hr. In some embodiments, the dye encapsulated in the lipid nanoparticle is heated at a temperature of about 65° C. for between about 18 hr to about 30 hr. In some embodiments, the dye encapsulated in the lipid nanoparticle is heated at a temperature of about 65° C. for between about 19 hr to about 30 hr. In some embodiments, the dye encapsulated in the lipid nanoparticle is heated at a temperature of about 65° C. for between about 20 hr to about 30 hr. In some embodiments, the dye encapsulated in the lipid nanoparticle is heated at a temperature of about 65° C. for between about 21 hr to about 30 hr. In some embodiments, the dye encapsulated in the lipid nanoparticle is heated at a temperature of about 65° C. for between about 22 hr to about 30 hr. In some embodiments, the dye encapsulated in the lipid nanoparticle is heated at a temperature of about 65° C. for between about 23 hr to about 30 hr. In some embodiments, the dye encapsulated in the lipid nanoparticle is heated at a temperature of about 70° C. for between about 14 hr to about 30 hr. In some embodiments, the dye encapsulated in the lipid nanoparticle is heated at a temperature of about 70° C. for between about 15 hr to about 30 hr. In some embodiments, the dye encapsulated in the lipid nanoparticle is heated at a temperature of about 70° C. for between about 16 hr to about 30 hr. In some embodiments, the dye encapsulated in the lipid nanoparticle is heated at a temperature of about 70° C. for between about 17 hr to about 30 hr. In some embodiments, the dye encapsulated in the lipid nanoparticle is heated at a temperature of about 70° C. for between about 18 hr to about 30 hr. In some embodiments, the dye encapsulated in the lipid nanoparticle is heated at a temperature of about 70° C. for between about 19 hr to about 30 hr. In some embodiments, the dye encapsulated in the lipid nanoparticle is heated at a temperature of about 70° C. for between about 20 hr to about 30 hr. In some embodiments, the dye encapsulated in the lipid nanoparticle is heated at a temperature of about 70° C. for between about 21 hr to about 30 hr. In some embodiments, the dye encapsulated in the lipid nanoparticle is heated at a temperature of about 70° C. for between about 22 hr to about 30 hr. In some embodiments, the dye encapsulated in the lipid nanoparticle is heated at a temperature of about 70° C. for between about 23 hr to about 30 hr. In some embodiments, the dye encapsulated in the lipid nanoparticle is heated at a temperature of about 75° C. for between about 14 hr to about 30 hr. In some embodiments, the dye encapsulated in the lipid nanoparticle is heated at a temperature of about 75° C. for between about 15 hr to about 30 hr. In some embodiments, the dye encapsulated in the lipid nanoparticle is heated at a temperature of about 75° C. for between about 16 hr to about 30 hr. In some embodiments, the dye encapsulated in the lipid nanoparticle is heated at a temperature of about 75° C. for between about 17 hr to about 30 hr. In some embodiments, the dye encapsulated in the lipid nanoparticle is heated at a temperature of about 75° C. for between about 18 hr to about 30 hr. In some embodiments, the dye encapsulated in the lipid nanoparticle is heated at a temperature of about 75° C. for between about 19 hr to about 30 hr. In some embodiments, the dye encapsulated in the lipid nanoparticle is heated at a temperature of about 75° C. for between about 20 hr to about 30 hr. In some embodiments, the dye encapsulated in the lipid nanoparticle is heated at a temperature of about 75° C. for between about 21 hr to about 30 hr. In some embodiments, the dye encapsulated in the lipid nanoparticle is heated at a temperature of about 75° C. for between about 22 hr to about 30 hr. In some embodiments, the dye encapsulated in the lipid nanoparticle is heated at a temperature of about 75° C. for between about 23 hr to about 30 hr.

In some embodiments, the method further comprises the step of adding a therapeutic agent. In some embodiments, the method comprises the step of encapsulating a therapeutic agent in the lipid nanoparticle. In some embodiments, the method comprises the step of attaching a therapeutic agent to the surface of the lipid nanoparticle. In various embodiments, the therapeutic agent is any therapeutic agent described herein.

In some embodiments, the method further comprises the step of adding a targeting domain. In some embodiments, the method further comprises the step of attaching a targeting domain to the surface of the lipid nanoparticle. In various embodiments, the targeting domain is any targeting domain described herein.

In some embodiments, the method further comprises the step of adding a pharmaceutically acceptable excipient.

### Method of Imaging

The present invention is further drawn to, in part, an imaging method comprising the steps of contacting a biological tissue with the compositions of the present invention, applying energy to a biological tissue comprising the composition, and imaging the biological tissue comprising the composition. In some embodiments, the composition further comprises at least one therapeutic agent.

In various embodiments, the method of imaging a biological tissue comprises application of at least one imaging technique. Examples of imaging techniques include, but are not limited to: photoacoustic imaging, ultrasound imaging, optical imaging, magnetic resonance imaging, computed tomography, thermal imaging, nuclear imaging, magnetomotive imaging enhancement, and any combination thereof.

In some embodiments, the biological tissue is a cancer cell. In some embodiments, the biological tissue is a tumor. In some embodiments, the biological tissue is tissue surrounding a tumor. In some embodiments, the biological tissue is a tumor and the tissue surrounding the tumor. In some embodiments, the biological tissue has high blood flow.

In some embodiments, the biological tissue experiences hypoxia. In some embodiments, the biological tissue is experiencing a stroke. In some embodiments, the tissue is experiencing diabetic ischemia. In some embodiments, the tissue is experiencing deep vein thrombosis. In some embodiments, the tissue is experiencing prenatal hypoxic-ischemic encephalopathy. In some embodiments, the tissue is experiencing preeclampsia. In some embodiments, the tissue is experiencing chronic obstructive pulmonary disease (COPD). In some embodiments, the tissue is in a cardiovascular disease state. In some embodiments, the tissue is in a neurological disease state. In some embodiments, the tissue is in a pulmonary disease state.

In some embodiments, the biological tissue is present in a mammal. In one embodiment, the biological tissue is present in mice. In one embodiment, the biological tissue is present in rats. In one embodiment, the biological tissue is present in humans.

In some embodiments, applying energy to a biological tissue comprises irradiating at least a portion of the biological tissue with a light source. In some embodiments, applying energy to a biological tissue comprises applying a radio frequency field. In some embodiments, applying energy to a biological tissue comprises exposing the biological tissue to irradiation at a wavelength between about 800 nm to about 1100 nm. In some embodiments, applying energy to a biological tissue comprises exposing the biological tissue to irradiation at a wavelength between about 850 nm to about 1100 nm. In some embodiments, applying energy to a biological tissue comprises exposing the biological tissue to irradiation at a wavelength between about 800 nm to about 890 nm. In some embodiments, applying energy to a biological tissue comprises exposing the biological tissue to irradiation at a wavelength between about 850 nm to about 900 nm. In some embodiments, applying energy to a biological tissue comprises exposing the biological tissue to irradiation at a wavelength between about 885 nm to about 995 nm. In some embodiments, applying energy to a biological tissue comprises exposing the biological tissue to irradiation at a wavelength between about 950 nm to about 1000 nm. In some embodiments, applying energy to a biological tissue comprises exposing the biological tissue to irradiation at a wavelength between about 1000 nm to about 1050 nm. In some embodiments, applying energy to a biological tissue comprises exposing the biological tissue to irradiation at a wavelength between about 1050 to 1100 nm. In some embodiments, applying energy to a biological tissue comprises exposing the biological tissue to irradiation at a wavelength between about 850 nm to about 1000 nm. In some embodiments, applying energy to a biological tissue comprises exposing the biological tissue to irradiation at a wavelength between about 850 nm to about 950 nm. In some embodiments, applying energy to a biological tissue comprises exposing the biological tissue to irradiation at a wavelength between about 900 nm to about 1100 nm. In some embodiments, applying energy to a biological tissue comprises exposing the biological tissue to irradiation at a wavelength between about 900 nm to about 1000 nm. In some embodiments, applying energy to a biological tissue comprises any combination of exposing the biological tissue to irradiation at any of the wavelength ranges thereof, irradiating at least a portion of the biological tissue with a light source, and applying a radio frequency field.

Ultrasound or cMUT transducers are used to detect the mechanically generated acoustic wave signals at the sample surface. The pressure field generated by the laser pulses and subsequently detected after interacting with heterogeneously absorbing and scattering tissue provides information about the spatial distribution of the absorbed electromagnetic energy. This permits mapping of the absorbed energy distribution within the tissue by its acoustic profile. The generation of sound waves by incident radiation is known as the “photoacoustic” or “optoacoustic” effect and is reviewed by Tam (Reviews of Modern Physics, 1986, 58(2), p381-431).

The incident radiation may be any type of energetic radiation, including electromagnetic radiation from radiofrequency to X-ray, electrons, protons, ions, and other particles. For simplicity, all of the above will be referred to herein as “radiation”. The word “light” will be used specifically to denote electromagnetic radiation of any wavelength or frequency. Preferred radiation is in the near IR spectrum, and may be generated by laser, microwave, etc.

Photoacoustic depth profiling can be performed when the measured sound wave is analyzed in terms of transit time from the site of light absorption back to the detector. Signals from deep within a sample take longer to reach the detector than those from regions near the surface. For pulsed irradiation the longer transit time translates into a larger separation between the time of arrival of the pulse and the arrival of the signal at the detector. For amplitude-modulated irradiation, the longer transit time translates into a phase change in the detected sound wave. Together photoacoustic microscopy and photoacoustic depth profiling constitute photoacoustic imaging.

The use of short bursts of light rather than continuously applied light may be helpful for photoacoustic depth profiling. In this case, the absorption of each light pulse and subsequent heating of the various regions of the sample produces one or more positive or negative pressure waves that propagate radially from the site of absorption after each pulse. For very short light pulses, the shape of the pressure pulses generated by the light pulses is determined by the optical and thermal properties, sizes and shapes of the different regions of the sample, as well as by the speed of sound within the sites and the surrounding medium (see for example, Karabutov et al., 1996, Appl. Phys., 63, p 545-563; Hutchins, 1986, Can. J. Phys., 64, p 1247-1264).

Contrast agents permit light absorption and sound generation in regions not otherwise possible. Contrast agents may also improve signal to noise ratio by increasing the amplitude of the sound wave. Increasing the sound wave amplitude allows an increase in the possible maximum depth of detection and thereby allows imaging of objects further below the surface of the body.

The present invention is drawn to, in part, a contrast agent that has a peak absorbance at a longer Near-IR wavelength (890 nm) than other similar agents. Longer wavelengths in the Near-IR penetrate deeper into biological tissue because molecules in these biological tissues absorb/scatter these wavelengths to a lesser extent, so not only do they increase to signal to noise, but they can also do it deeper in biological tissues. Thus these particles also enable more effective photoacoustic imaging at longer NIR wavelengths.

The use of contrast media provides significant amplification of the signal strength, and thus permits improved imaging. Such a contrast agent for photoacoustic imaging works by either (i) enhancing the pre-existing photoacoustic effect or (ii) creating a photoacoustic effect where this was previously not possible. This may be achieved by selectively absorbing radiation in certain organs or healthy or diseased bodily structures or parts thereof, and/or by efficiently converting the radiation into heat, and/or by facilitating or improving heat-pressure conversion, and/or by scattering and diffusing the incident light so that it more uniformly illuminates the target organs.

Tissue of particular interest for imaging include, without limitation, tissues not shielded by bone, e.g., breast tissue, liver tissue: etc.; and blood vessels, which have been found to provide for unexpected amplification of signal. Subjects of interest for imaging include those suspected or know to have liver cancer, breast cancer, atherosclerosis, soft tissue sarcomas, and the like.

The preferred dosage of the contrast media will vary according to a number of factors, such as the administration route, the age, weight and species of the subject, but in general containing in the order of from 1 μmol/kg to 1 mmol/kg bodyweight of the contrast agent.

Administration may be parenteral (e.g., intravenously, intraarterially, intramuscularly, interstitially, subcutaneously, transdermally, or intrasternally) or into an externally voiding body cavity (e.g., the gastrointestinal tract, bladder, uterus, vagina, nose, ears or lungs), in an animate human or non-human (e.g., mammalian, reptilian or avian) body. Usually administration is accomplished by intravenous or intratumor injection.

Imaging of the desired area is performed by detection and appropriate analysis of the sound waves resulting from irradiation. Detection may be performed at the same surface of the sample as the source of incident radiation (reflection) or alternatively at another surface such as the surface diametrically opposed to the incident light, i.e., the sample's back surface (transmission). Suitable methods of detection include the use of a microphone, piezoelectric transducer, capacitance transducer, fiber-optic sensor, cMUT, or alternatively non-contact methods (see Tam, 1986, supra for a review). Techniques and equipment used in ultrasound imaging may be used.

The methods and uses described herein are especially useful for imaging blood-containing structures (e.g., blood vessels), which may be present in tumors, diseased tissue or particular organs, by the use of contrast agents with specificity for that region/structure, e.g., by use of biological recognition agents with the desired specificity.

Continuous wave radiation may be used with its amplitude or frequency modulated. When continuous wave radiation is used, the photoacoustic effects may be analyzed in the frequency domain by measuring amplitude and phase of one or several Fourier components. Alternatively, and preferably, short pulses (impulses) of radiation are employed which allow stress confinement. When pulses are used, analysis may be made in the time domain, i.e., on the basis of the time taken for the sound wave to reach the detector, thus simplifying analysis and aiding depth profiling.

Information obtained from the methods of the invention described herein can be used alone, or in combination with other information (e.g., age, family history, disease status, disease history, vital signs, blood chemistry, PSA level, Gleason score, lymph node staging, metastasis staging, expression of other gene signatures relevant to outcomes of a disease or disorder, such as cancer, etc.) from the subject or from the biological sample obtained from the subject. In some embodiments, the imaging data is combined or correlated with other data or test results that include, but are not limited to measurements or results from serologic testing methods, enzyme immunoassay (EIA), complement fixation (CF), immunodiffusion, clinical presentation, serology, radiography, histology, culture, and clinical parameters or other algorithms for developing or having a disease or disorder, such as cancer. In some embodiments, data include, but are not limited to age, ethnicity, PSA level, Gleason score, lymph node staging, metastasis staging, and other genomic data, and specific expression values of other gene signatures relevant to infection outcomes. In some embodiments, the data comprises subject information, such as medical history, travel history, and/or any relevant family history. Several serology techniques that can be used in combination with the compositions and methods of the present invention. Examples of serology techniques include, but are not limited to: ELISA, agglutination, precipitation, complement-fixation, fluorescent antibodies, and chemiluminescence.

In some embodiments, imaging the biological tissue comprises transducing the resulting ultrasound signal from the biological tissue and producing an image in a data processor from the transduced ultrasound signal.

In some embodiments, the imaging method further comprises the step of allowing the composition to accumulate in a region of the biological tissue. In some embodiments, the imaging method further comprises the step of allowing the composition to accumulate in a region of the biological tissue wherein the targeting domain facilitates accumulation of the composition in the region. In one embodiment, the composition accumulates around a region of biological tissue.

In various aspects, the composition of the present invention may be delivered to a cell or biological tissue of interest by a red-blood cell-hitchhiking methods that are well-known to those of skill in the art, and such methods are incorporated herein by reference. Examples of such red-blood cell-hitchhiking methods are described in Brenner et al., 2018, Nature Commun., 9:2684.

In various embodiments, the clearance, excretion rates, and pathways of the lipid nanoparticles can be predetermined by the size (e.g., average diameter) of the lipid nanoparticles and physiochemical properties of capping ligands and templating polymers. This approach provides a flexible platform for designing and validation of various types of nanoparticles for safe clinical use. For example, different types of nanomaterials can be clustered together providing multiplexing opportunities for synthesis of multifunctional/multimodal nanoparticles.

In some embodiments, further applications include therapeutic agent encapsulation inside the lipid nanoparticles with controlled release that can be triggered by one of the following stimulus. For example, in some embodiments, a pH change is applied to a lipid nanoparticle comprising a therapeutic agent, wherein the pH change causes the release of the therapeutic agent from the lipid nanoparticle. In some embodiments, this may provide a clinician the ability to control and visualize drug therapy noninvasively.

### Method of Treatment and Delivery

The present invention is further drawn to, in part, a method of treating a disease or disorder in a subject in need thereof, wherein the method comprises administering a therapeutically effective amount of the composition of the present invention to the subject.

In various aspects, the present invention is further drawn to, in part, a method of administering at least one composition of the present invention to a subject. In some embodiments, the subject was diagnosed with cancer.

In various aspects, the present invention relates to, in part, a phototherapy method, wherein the method comprises administering a therapeutically effective amount of the composition of the present invention to the subject and irradiating the subject at a wavelength of at or above about 800 nm. In some embodiments, the method comprises administering a therapeutically effective amount of the composition of the present invention to the subject and irradiating the subject at a wavelength of at or above about 850 nm.

In some embodiments, the method comprises irradiating the subject at a wavelength of between about 800 nm to about 1100 nm. In some embodiments, the method comprises irradiating the subject at a wavelength of between about 850 nm to about 1100 nm. In some embodiments, the method comprises irradiating the subject at a wavelength of between about 800 nm to about 850 nm. In some embodiments, the method comprises irradiating the subject at a wavelength of between about 800 nm to about 890 nm. In some embodiments, the method comprises irradiating the subject at a wavelength of between about 850 nm to about 900 nm. In some embodiments, the method comprises irradiating the subject at a wavelength of between about 880 nm to about 900 nm. In some embodiments, the method comprises irradiating the subject at a wavelength of between about 885 nm to about 895 nm. In some embodiments, the method comprises irradiating the subject at a wavelength of between about 950 nm to about 1000 nm.

In some embodiments, the subject is irradiated at a wavelength of at or above 800 nm. In some embodiments, the subject is irradiated at a wavelength of at or above 810 nm. In some embodiments, the subject is irradiated at a wavelength of at or above 820 nm. In some embodiments, the subject is irradiated at a wavelength of at or above 830 nm. In some embodiments, the subject is irradiated at a wavelength of at or above 840 nm. In some embodiments, the subject is irradiated at a wavelength of at or above 860 nm. In some embodiments, the subject is irradiated at a wavelength of at or above 870 nm. In some embodiments, the subject is irradiated at a wavelength of at or above 880 nm. In some embodiments, the subject is irradiated at a wavelength of at or above 885 nm. In some embodiments, the subject is irradiated at a wavelength of at or above 886 nm. In some embodiments, the subject is irradiated at a wavelength of at or above 887 nm. In some embodiments, the subject is irradiated at a wavelength of at or above 888 nm. In some embodiments, the subject is irradiated at a wavelength of at or above 889 nm. In some embodiments, the subject is irradiated at a wavelength of at or above 890 nm. In some embodiments, the subject is irradiated at a wavelength of at or above 891 nm. In some embodiments, the subject is irradiated at a wavelength of at or above 892 nm. In some embodiments, the subject is irradiated at a wavelength of at or above 893 nm. In some embodiments, the subject is irradiated at a wavelength of at or above 894 nm. In some embodiments, the subject is irradiated at a wavelength of at or above 895 nm. In some embodiments, the subject is irradiated at a wavelength of at or above 900 nm.

In some embodiments, the composition further comprises a targeting domain.

In some embodiments, the composition further comprises at least one therapeutic agent.

In some embodiments, the composition or the lipid nanoparticle of the present invention selectively targets at least one cell of interest. In some embodiments, the composition or the lipid nanoparticle of the present invention selectively binds to at least one cell of interest.

In one embodiment, the composition or the lipid nanoparticle induces cell death of at least one cell of interest.

In some embodiment, the irradiating the subject at a wavelength of at or above about 850 nm selectively heats up at least one cell of interest. In some embodiment, the irradiating the subject at a wavelength of at or above about 850 nm selectively heats up at least one cell of interest that is selectively targeted by and/or bound to the composition or the lipid nanoparticle.

In some embodiments, the irradiation selectively induces cell death of at least one cell of interest. In some embodiments, the irradiation selectively kills at least one cell of interest. In some embodiments, the irradiation selectively kills at least one cell of interest that is selectively targeted by and/or bound to the composition or the lipid nanoparticle. In some embodiments, the irradiation selectively kills at least one cell of interest that is selectively targeted by and/or bound to the composition or the lipid nanoparticle by heating the composition or the lipid nanoparticle.

In some embodiments, the irradiation selectively inhibits at least one cell of interest. In some embodiments, the irradiation selectively inhibits at least one cell of interest that is selectively targeted by and/or bound to the composition or the lipid nanoparticle. In some embodiments, the irradiation selectively inhibits at least one cell of interest that is not selectively targeted by the composition or the lipid nanoparticle. In some embodiments, the irradiation selectively causes at least one cell of interest that is selectively targeted by and/or bound to the composition or the lipid nanoparticle to release cell messengers that begin at least one signaling pathway. In some embodiments, the irradiation selectively causes at least one cell of interest that is selectively targeted by and/or bound to the composition or the lipid nanoparticle to cause at least one anti-tumor cellular response. In some embodiments, the irradiation selectively causes at least one cell of interest that is selectively targeted by and/or bound to the composition or the lipid nanoparticle to cause at least one other cell to go through apoptosis. In some embodiments, the irradiation selectively causes at least one cell of interest that is selectively targeted by and/or bound to the composition or the lipid nanoparticle to cause at least one other cell to experience increased immune action. In some embodiments, the irradiation selectively causes at least one cell of interest that is selectively targeted by and/or bound to the composition or the lipid nanoparticle to cause at least one other cell to experience decreased immune action. In some embodiments, the irradiation selectively causes at least one cell of interest that is selectively targeted by and/or bound to the composition or the lipid nanoparticle to cause at least one other cell to elicit an anti-angiogenic response. In some embodiments, the irradiation selectively inhibits at least one cell of interest that is selectively targeted by and/or bound to the composition or the lipid nanoparticle by heating the composition or the lipid nanoparticle.

In some embodiments, the cell of interest is a cancer cell. In some embodiments, the cell of interest is a cancer associated fibroblast (CAF). In some embodiments, the cell of interest is a tumor associated microphage (TAM). In some embodiments, the cell of interest is a dendritic cell. In some embodiments, the cell of interest is a cell which exists in a tumor microenvironment.

The following are non-limiting examples of cancers that can be imaged, detected, and/or treated by the disclosed methods and compositions: acute lymphoblastic; acute myeloid leukemia; adrenocortical carcinoma; adrenocortical carcinoma, childhood; appendix cancer; basal cell carcinoma; bile duct cancer, extrahepatic; bladder cancer; bone cancer; osteosarcoma and malignant fibrous histiocytoma; liposarcoma and anaplastic liposarcoma; brain stem glioma, childhood; brain tumor, adult; brain tumor, brain stem glioma, childhood; brain tumor, central nervous system atypical teratoid/rhabdoid tumor, childhood; central nervous system embryonal tumors; cerebellar astrocytoma; cerebral astrocytotna/malignant glioma; craniopharyngioma; ependymoblastoma; ependymoma; medulloblastoma; medulloepithelioma; pineal parenchymal tumors of intermediate differentiation; supratentorial primitive neuroectodermal tumors and pineoblastoma; visual pathway and hypothalamic glioma; brain and spinal cord tumors; breast cancer; bronchial tumors; Burkitt lymphoma; carcinoid tumor; carcinoid tumor, gastrointestinal; central nervous system atypical teratoid/rhabdoid tumor; central nervous system embryonal tumors; central nervous system lymphoma; cerebellar astrocytoma cerebral astrocytoma/malignant glioma, childhood; cervical cancer; chordoma, childhood; chronic lymphocytic leukemia; chronic myelogenous leukemia; chronic myeloproliferative disorders; colon cancer; colorectal cancer; craniopharyngioma; cutaneous T-cell lymphoma; esophageal cancer; Ewing family of tumors; extragonadal germ cell tumor; extrahepatic bile duct cancer; eye cancer, intraocular melanoma; eye cancer, retinoblastoma; biliary track cancer, cholangiocarcinoma, anal cancer, neuroendocrine tumors, small bowel cancer, gallbladder cancer; gastric (stomach) cancer; gastrointestinal carcinoid tumor; gastrointestinal stromal tumor (gist); germ cell tumor, extracranial; germ cell tumor, extragonadal; germ cell tumor, ovarian; gestational trophoblastic tumor; glioma; glioma, childhood brain stem; glioma, childhood cerebral astrocytoma; glioma, childhood visual pathway and hypothalamic; hairy cell leukemia; head and neck cancer; hepatocellular (liver) cancer; histiocytosis, langerhans cell; Hodgkin lymphoma; hypopharyngeal cancer; hypothalamic and visual pathway glioma; intraocular melanoma; islet cell tumors; kidney (renal cell) cancer; Langerhans cell histiocytosis; laryngeal cancer; leukemia, acute lymphoblastic; leukemia, acute myeloid; leukemia, chronic lymphocytic; leukemia, chronic myelogenous; leukemia, hairy cell; lip and oral cavity cancer; liver cancer; lung cancer, non-small cell; lung cancer, small cell; lymphoma, aids-related; lymphoma, burkitt; lymphoma, cutaneous T-cell; lymphoma, non-Hodgkin lymphoma; lymphoma, primary central nervous system; macroglobulinemia, Waldenstrom; malignant fibrous histiocvtoma of bone and osteosarcoma; medulloblastoma; melanoma; melanoma, intraocular (eye); Merkel cell carcinoma; mesothelioma; metastatic squamous neck cancer with occult primary; mouth cancer; multiple endocrine neoplasia syndrome, (childhood); multiple myeloma/plasma cell neoplasm; mycosis; fungoides; myelodysplastic syndromes; myelodysplastic/myeloproliferative diseases; myelogenous leukemia, chronic; myeloid leukemia, adult acute; myeloid leukemia, childhood acute; myeloma, multiple; myeloproliferative disorders, chronic; nasal cavity and paranasal sinus cancer; nasopharyngeal cancer; neuroblastoma; non-small cell lung cancer; oral cancer; oral cavity cancer; oropharyngeal cancer; osteosarcoma and malignant fibrous histiocytoma of bone; ovarian cancer; ovarian epithelial cancer; ovarian germ cell tumor; ovarian low malignant potential tumor; pancreatic cancer, islet cell tumors; papillomatosis; parathyroid cancer; penile cancer; pharyngeal cancer; pheochromocytoma; pineal parenchymal tumors of intermediate differentiation; pineoblastoma and supratentorial primitive neuroectodermal tumors; pituitary tumor; plasma celt neoplasm/multiple myeloma; pleuropulmonary blastoma; primary central nervous system lymphoma; prostate cancer; rectal cancer; renal cell (kidney) cancer; renal pelvis and ureter, transitional cell cancer; respiratory tract carcinoma involving the nut gene on chromosome 15; retinoblastoma; rhabdomyosarcoma; salivary gland cancer; sarcoma, ewing family of tumors; sarcoma, Kaposi; sarcoma, soft tissue; sarcoma, uterine; Sezary syndrome; skin cancer (nonmelanoma); skin cancer (melanoma); skin carcinoma, Merkel cell; small cell lung cancer; small intestine cancer; soft tissue sarcoma; squamous cell carcinoma, squamous neck cancer with occult primary, metastatic; stomach (gastric) cancer; supratentorial primitive neuroectodermal tumors; T-cell lymphoma, cutaneous; testicular cancer; throat cancer; thymoma and thymic carcinoma; thyroid cancer; transitional cell cancer of the renal pelvis and ureter; trophoblastic tumor, gestational; urethral cancer; uterine cancer, endometrial; uterine sarcoma; vaginal cancer; vulvar cancer; Waldenstrom macroglobulinemia; and Wilms tumor.

In one aspect, the amount of cells associated with cancer are used to monitor subjects undergoing treatments and therapies for a cancer, subjects who have had a cancer, and subjects who are in remission of a previously diagnosed and treated cancer. In some embodiments, the amount of cells associated with a cancer are used to select or modify treatments in subjects having a cancer, subjects who have had a cancer, and subjects who are in remission of a previously diagnosed and treated cancer.

In some embodiments, the method of treating further comprises the step of allowing the composition to accumulate in at least one cell of interest, wherein the targeting domain facilitates accumulation of the composition in the at least one cell of interest. In one embodiment, the composition accumulates around the at least one cell of interest. In one embodiment, the composition accumulates in the blood stream around the at least one cell of interest.

In one aspect, the present invention also provides methods for identifying agents for treating a disease or disorder that are appropriate or otherwise customized for a specific subject. In some embodiments, a test sample from a subject, exposed to a therapeutic agent or a drug, can be taken and the amount of cells associated with a disease or disorder can be determined. In some embodiments, the amount of cells associated with a disease or disorder can be compared to a sample derived from the subject before and after treatment, or can be compared to samples derived from one or more subjects who have shown improvements or alleviation of a disease or disorder as a result of such treatment or exposure.

To identify therapeutic agents that are appropriate for a specific subject, a test sample from the subject can also be exposed to a therapeutic agent or a drug, and the amount of cells associated with a disease or disorder can be determined. The amount of cells associated with a disease or disorder can be compared to a sample derived from the subject before and after treatment or exposure to a therapeutic agent or a drug, or can be compared to samples derived from one or more subjects who have shown improvements relative to a disease as a result of such treatment or exposure. Thus, in one aspect, the invention provides a method of assessing the efficacy of a therapy with respect to a subject comprising a step of taking a first measurement of the amount of cells associated with a disease or disorder in a first sample from the subject; a step of effecting the therapy with respect to the subject; a step of taking a second measurement of the amount of cells associated with a disease or disorder in a second sample from the subject; and a step of comparing the first and second measurements to assess the efficacy of the therapy.

In various exemplary embodiments, the methods of the invention include effecting a therapy for the treatment of a diagnosed disease. In some embodiments, effecting a therapy comprises administering a disease-modulating therapeutic agent to the subject. In various embodiments, effecting a therapy comprises treatment of one or more symptoms of the disease or disorder. For example, in some embodiments, effecting a therapy comprises administration of a non-disease-modulating drug to the subject. Exemplary non-disease-modulating drugs that may be administered include, but are not limited to, pain relievers, anti-inflammatory drugs, NSAIDs, decongestants, cough suppressants, including topical cough suppressants, or other agents that may function to reduce the severity of at least one symptom of the disease or disorder.

Any therapeutic agent or any combination of therapeutic agents disclosed herein may be administered to a subject to treat a disease or disorder. The therapeutic agents herein can be formulated in any number of ways, often according to various known formulations in the art or as disclosed or referenced herein.

In various embodiments, one or more additional drugs may be optionally administered in addition to those that are recommended or have been administered. An additional drug will typically not be any drug that is not recommended or that should be avoided.

In various aspects, the lipid nanoparticle or compositions thereof of the present invention can be used alone or in combination with a therapeutic agent to deliver a therapeutic agent payload to a target cell. Often, the therapeutic agent may be released based on the degradation of, e.g., a controlled biodegradation of the lipid nanoparticle.

In one aspect, the present invention discloses a method by which a therapeutic agent can be delivered to a subject in need of a therapeutic agent. In some embodiments, the biodegradable nanoparticle composition of the present invention that are administered to the subject comprises one or more therapeutic agents. In various embodiments, the therapeutic agent is released upon biodegradation of the lipid nanoparticles.

Thus, in various aspects, the present invention also provides a method for delivering a therapeutic agent comprising the steps of: identifying a subject in need of the therapeutic agent; administering the therapeutic agent; wherein the therapeutic agent is associated with a biodegradable lipid nanoparticle comprising one or more lipid nanoparticles and one or more stabilizers; and releasing the therapeutic agent by biodegrading the lipid nanoparticles. In certain aspects, the biodegradable nanoparticle comprises a hydrodynamic diameter smaller than 200 nm and has an absorbance in the NIR window between 850 nm and 1100 nm.

The lipid nanoparticles or compositions thereof of the present invention can be adapted for administration using a wide variety of methods of delivery, including, but not limited to, e.g., subcutaneous, intravenous, peritoneally, orally, intramuscular, topical, nasally, intradermal, ocular, rectal, vaginal and combinations thereof.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

Liposomal J-Aggregates (L-JA) of Indocyanine Green (IcG) can serve as a biocompatible and biodegradable nanoparticle for photoacoustic imaging and photothermal therapy. When compared to monomeric IcG, L-JA are characterized by longer circulation, improved photostability, elevated absorption at longer wavelengths, and increased photoacoustic signal generation. However, the documented methods for production of L-JA vary widely. The present invention was developed as an approach to efficiently form IcG J-aggregates (IcG-JA) directly in liposomes at elevated temperatures. Aggregating within fully formed liposomes ensured particle uniformity and allowed for control of J-aggregate size. L-JA had unique properties compared to IcG. L-JA provided significant contrast enhancement in photoacoustic images for up to 24 hours after injection, while IcG and unencapsulated IcG-JA were cleared within an hour. L-JA allowed for more accurate photoacoustic-based sO₂ estimation and particle tracking compared to IcG. Furthermore, photothermal heating of L-JA with an 852 nm laser was demonstrated to be more effective than conventional 808 nm lasers for the first time. The present technique offers an avenue for formulating a multi-faceted contrast agent for photoacoustic imaging and photothermal therapy that offers significant advantages over other conventional agents.

IcG-JA alone are promising contrast agents (Liu, R. et al., 2017, Nanotheranostics, 1:430; Vincy, A. et al., 2022, ACS Biomater. Sci. Eng., 8, 5119), and combining the aggregates with biocompatible lipid, polymer, or inorganic nanoparticles can broaden their utility (Changalvaie, B. et al., 2019, ACS Appl. Mater. Interfaces, 11:46437; Wood, C. A. et al., 2021, Nat. Commun., 12, 5410; Cheung, C. C. L. et al., 2020, Nanotheranostics, 4:91; Xu, W. et al., 2023, Photoacoustics, 33:100552). Nanoparticles can assist with particle homogeneity as it is difficult to control the size of IcG-JA when they are allowed to grow without constraints. When not restricted, IcG-JA can grow to sizes that span hundreds of nanometers (Singh, S. et al., 2022, Photoacoustics, 29, 100437) making them less attractive for in vivo applications. Moreover, nanoparticles offer a variety of advantages with regard to biocompatibility, circulation time, co-encapsulation with a therapeutic agent, and the ability to add targeting motifs. With a peak absorbance near 890 nm, IcG-JA are relatively unique as few nanoparticle-based contrast agents in the first NIR window have peaks above 850 nm. Reaching these peak wavelengths typically requires metallic nanoparticles such as gold, silver, zinc, and copper (Min, K. H. et al., 2017, Theranostics, 7:4240; Choi, H. et al., 2019, Biomacromolecules, 20:3767; Marek, M. R. J. et al., 2022, ACS Omega, 7:36653). While promising, metallic contrast agents are often characterized by high levels of photodegradation (Cavigli, L. et al., 2021, Nanomaterials (Basel), 11:116; Zhang, Q. et al., 2018, Nat. Commun., 9:1183; Ungureanu, C. et al., 2011, Nano Lett., 11:1887; Soltani, R. D. C. et al., 2015, Desalination and Water Treatment, 56:2551) and there are mixed reports on long-term biocompatibility and biodegradability (Balfourier, A. et al., 2020, Proc. Natl. Acad. Sci. USA, 117:103; Kolosnjaj-Tabi, J. et al., 2015, ACS Nano, 9:7925; Yang, Y. et al., 2022, J. Nanobiotechnology, 20, 37; Vimercati, L. et al., 2020, Frontiers in Public Health, 8; Xiong, P. et al., 2022, Advanced Science, 9:2106049; Sani, A. et al., 2021, Biochem. Biophys. Rep., 26:100991). Beyond diagnostic applications, an 890 nm peak suggests PTT at longer wavelengths may be possible, yet most documented studies on J-aggregates are limited to 808 nm (Liu, R. et al., 2017, Nanotheranostics, 1:430; Cheung, C. C. L. et al., 2020, Nanotheranostics, 4:91; Xu, W. et al., 2023, Photoacoustics, 33:100552; Kwon, N. et al., 2023, Angewandte Chemie International Edition, 62:e202305564; Millard, M. et al., 2023, Colloids and Surfaces B: Biointerfaces, 230:113516). The advantages of increased depth and limited tissue attenuation offered by longer wavelengths also apply for therapeutic applications. By limiting IcG-JA based photothermal therapies to 808 nm, the full potential of these particles for effective therapy can not be achieved. As such, IcG-JA based nanoparticles are not only promising for imaging applications, but also may be an excellent contrast agent for therapy at much longer wavelengths.

Methods for encapsulating pre-formed IcG-JA have been moderately successful; however, batch-to-batch variability, low encapsulation efficiencies, and potentially months-long synthesis times hinder translation. Thus, there exists a continuing need to optimize the encapsulation of IcG-JA within nanoparticles and investigate their use for multimodal imaging and therapy. The invention described herein is a straightforward procedure that utilizes IcG to rapidly produce consistent liposome-encapsulated J-Aggregates of IcG (L-JA) with the use of readily available high-transition temperature lipids. IcG is first encapsulated in liposomes using conventional techniques followed by extrusion just above the transition temperature of the lipids. The encapsulated liposomes are then subjected to heating at an optimal temperature to allow IcG-JA to form within liposomes over a relatively short period of time. With this synthesis as the focus, the objective of this study was to develop this time-efficient method for the generation of L-JA and to characterize the optical, photoacoustic, and photothermal properties of these L-JA.

### Example 1: A Facile Approach to Producing Liposomal J-Aggregates of Indocyanine Green for Photoacoustic Imaging and Molecular Imaging

Nanoparticles can assist with particle homogeneity as it is difficult to control the size of IcG-JA when they are allowed to grow unhindered and nanoparticles offer a variety of advantages with regards to biocompatibility, circulation time, co-encapsulation with a therapeutic agent, and the ability to add targeting motifs.

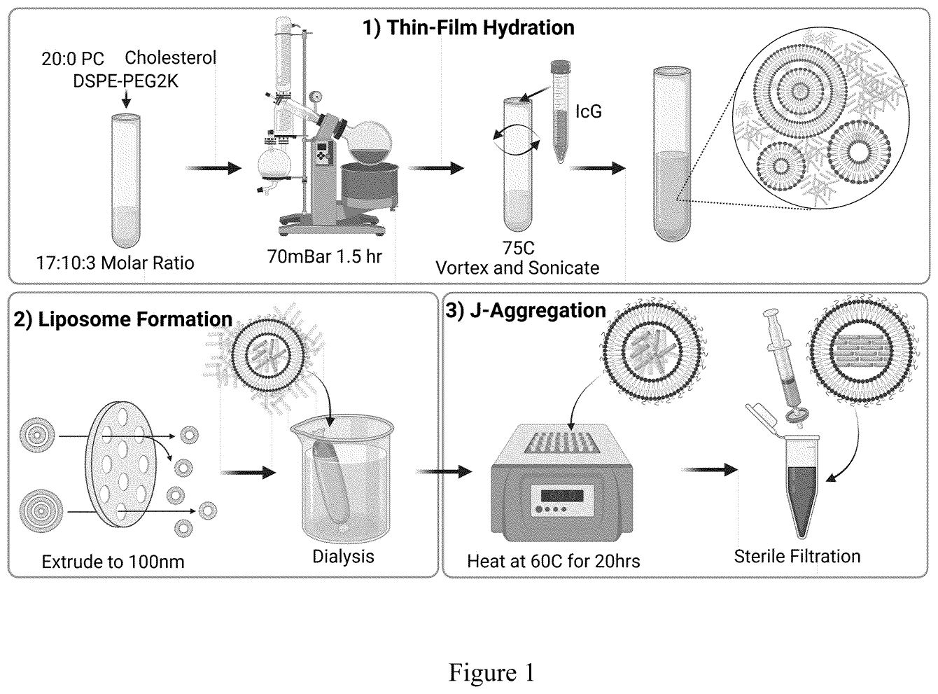

The present invention provides a straightforward procedure that used FDA-approved materials and techniques to rapidly produce consistent liposome-encapsulated J-Aggregates of IcG (L-JA) with the use of readily available high-transition temperature lipids as the bulk of the liposomal shell (FIG. 1). High-transition temperature lipids allowed the herein synthesized particles to withstand the high heat required to rapidly produce IcG-JA, but otherwise had very little difference to other commonly used lipids. High transition temperatures are essential as they ensure that particles will maintain stability at elevated temperatures during extrusion and most importantly, that the particles can withstand the 60° C. temperature required for efficient J-aggregation. Substituting for more conventional lower transition temperature lipids increases the risk of IcG leakage during extrusion and complete liposomal collapse during the day-long heating process. With no apparent change to biocompatibility and stability, and only minor adjustments to synthesis procedures, higher transition temperature lipids are ideally suited for this process and are the main reason L-JA can consistently be produced in as little as 3 days. Using conventional liposome formation techniques followed by extrusion at high temperatures and heating at an optimal temperature, high concentrations of monomeric IcG were first encapsulated and then forced to aggregate in the confined space of the liposome. The goal of the investigation was to demonstrate this technique produces viable liposomal nanoparticles that are not only comparable to other liposomes but are also superior to IcG and non-encapsulated free IcG-JA. The liposomal J-Aggregates can be used in photoacoustic functional imaging and molecular imaging.

The present method is faster and more reproducible than previously developed methods to produce liposomal J-Aggregates. The most well-known formulation for Liposomal J-Aggregates of IcG involves encapsulating IcG first and allowing the aggregation to occur slowly over at least 60 days. The present process relies on high transition temperature lipids (20:0 PC) in which IcG is first encapsulated in a similar manner to other procedures, but then heated to rapidly cause J-Aggregation on the pre-encapsulated IcG without destabilizing the liposome. In the end, what was previously a 60+ day procedure can be done in 3 days. This specific particle formulation also allows for improved photothermal therapy efficacy.

Photothermal therapy, which relies on high power laser exposures, is a minimally invasive technique that has promising tumor treatment applications. When irradiated with a continuous laser, these particles have the potential to locally heat tissue to temperatures that can ablate cells. Generally, photothermal therapy is performed using a single wavelength near-infrared laser. For IcG applications, laser wavelengths are typically either 780 nm or 808 nm to capitalize on the peak absorption of IcG in this range. However, hemoglobin is a strong absorber at these wavelengths. Consequently, blood attenuates the transmitted light and limits the depth at which photothermal therapy is useful. The J-Aggregates of IcG disclosed herein have peak absorption at 890 nm where blood is less attenuative. Thus, Liposomal J-aggregates of IcG disclosed in the present invention enable photothermal therapy at deeper depths in tissue and solid tumors.

Overall, this novel approach to creating photothermally active particles combined with a novel laser irradiation strategy is a promising cancer treatment technique. Applications exist for a wide variety of cancers, including but not limited to brain, head and neck, breast, and prostate. While designed with cancer treatment as the main goal, treatment with this novel technique could be applied to any disease where photothermal therapy is viable.

The present invention involves the use of high transition temperature lipids, such as 20:0 PC which has a transition temperature above the 60° C. temperature needed for rapid and efficient IcG J-Aggregate formation. Metallic nanoparticles, particularly gold nanoparticles, are typically used for photothermal therapy. While gold is biologically inert, gold nanoparticles are not biodegradable and may reside in the body indefinitely. The IcG J-aggregates of the present invention are biodegradable and thus can be cleared from the body through established metabolic pathways. For photothermal therapy, using laser wavelengths higher than 850 nm allows for photothermal therapy with lower optical intensities and deeper in tissue.

High transition temperature lipids such as 20:0 PC allow for aggregation of IcG dispersed in the liposomal liquid core at elevated temperatures. Once the liposomes are formed and extruded to a desired average diameter, they can withstand the 60° C. temperature needed to initiate efficient IcG J-aggregation. Lipids that are commonly used in liposomes, such as such as DSPC or DOPC, have a melting temperature below 60° C.; consequently, IcG would leak out of these liposomes when heated to 60° C. A higher transition temperature is the key element that allows for significant reduction of the time for J-aggregate formation from 60+ days to approximately 3 days.

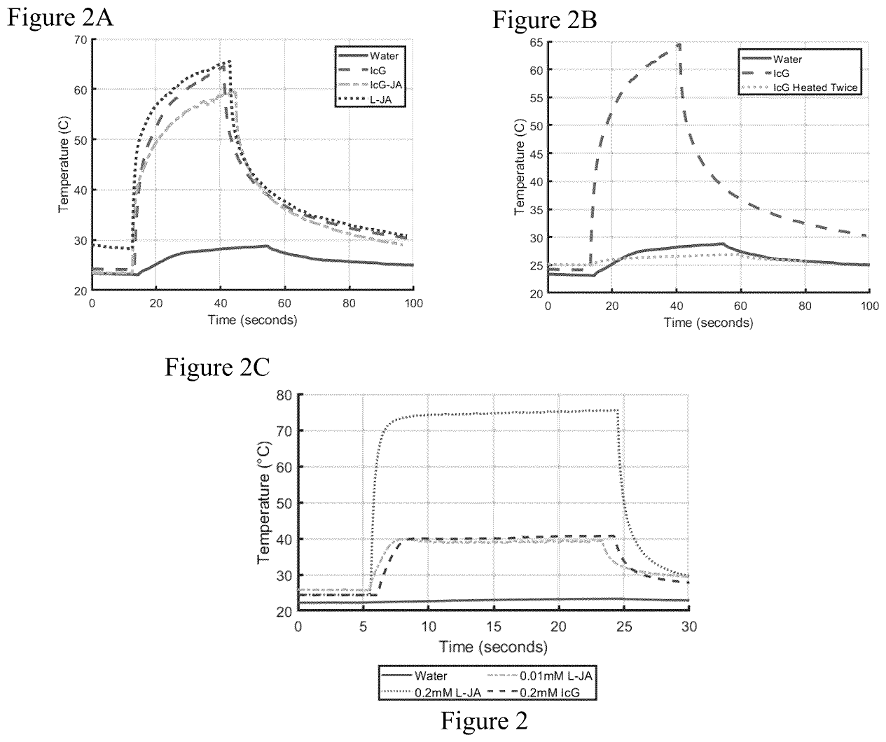

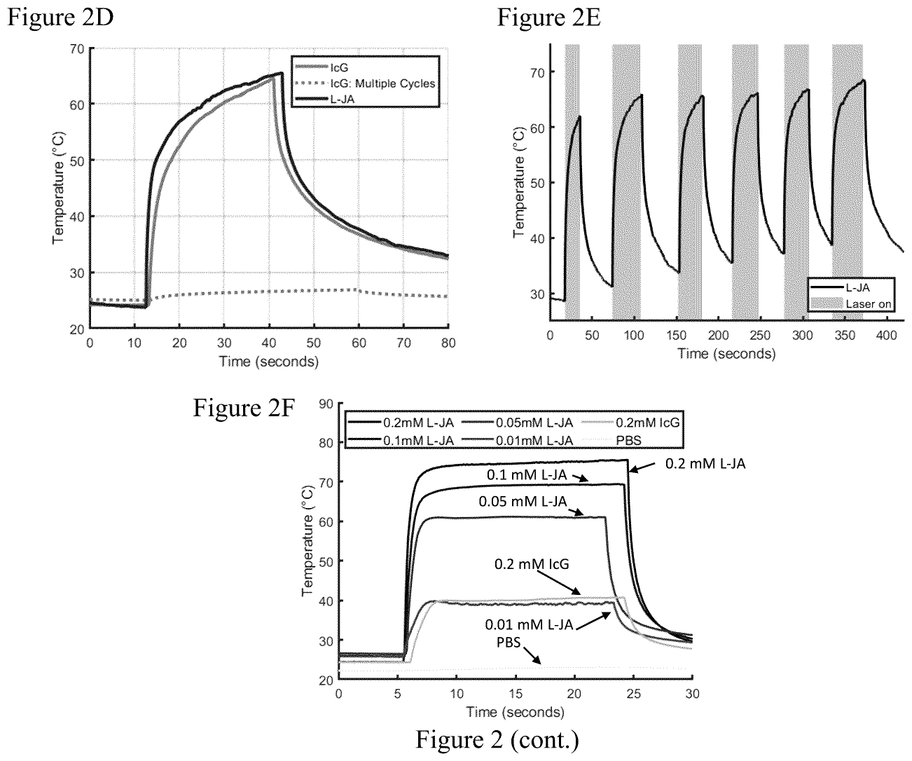

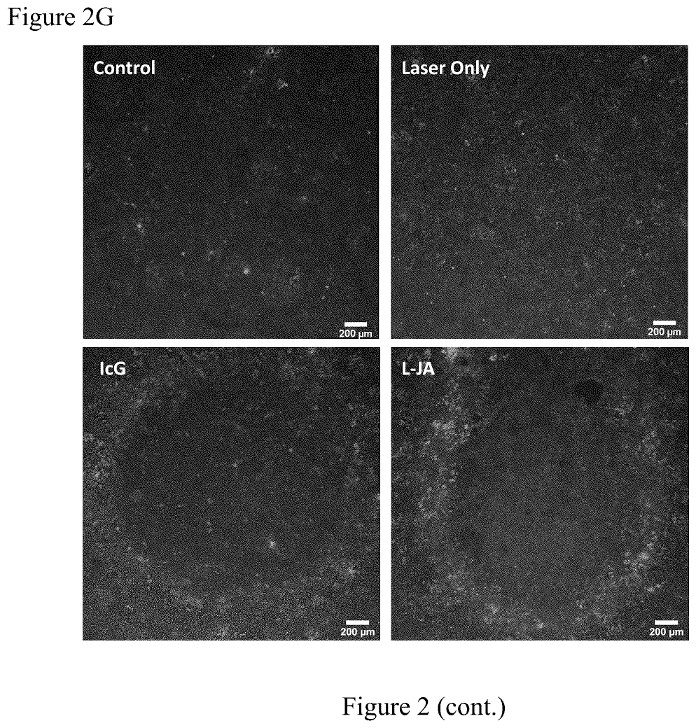

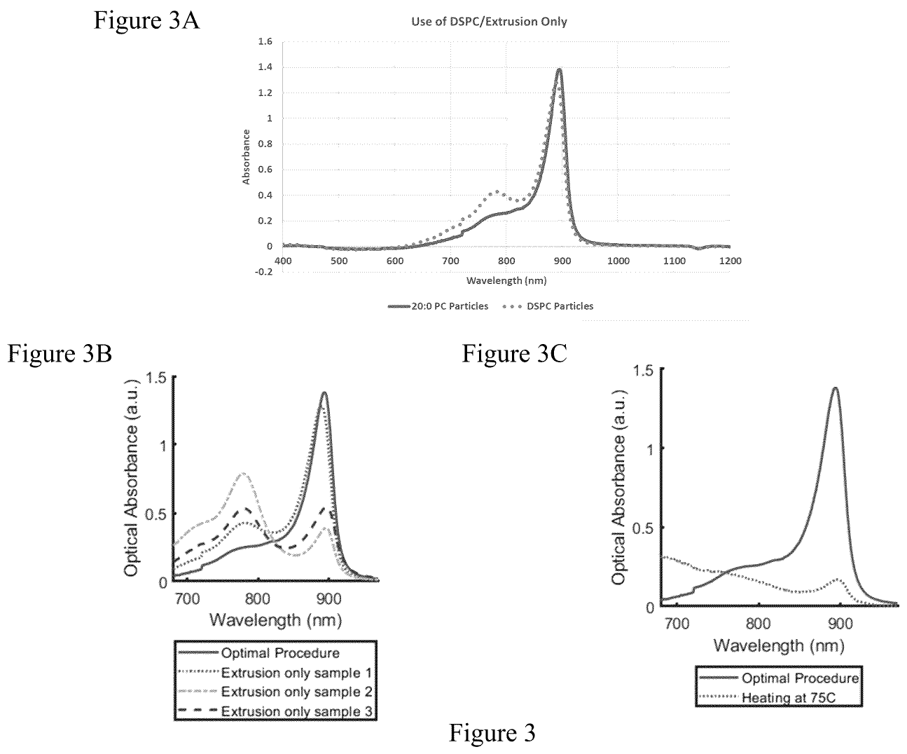

The use of longer wavelength lasers with wavelengths at or above 850 nm, provides a host of advantages over the more conventional 808 nm laser. First, as wavelengths increase towards the J-Aggregate peak absorption of 890 nm, photon absorption and heat generation increase dramatically. This means that a lower concentration of particles can achieve the same level of heating that nanoparticles that absorb at 808 nm would provide. Notably, J-Aggregates of IcG and their liposomal form can repeatedly reach temperatures of 60° C. and above, while IcG can only be heated once (FIG. 2A, FIG. 2B). Unlike heating with a 808 nm laser, at equal concentrations the L-JA solution reaches significantly higher temperatures than IcG. Like with the 808 nm laser, when heated with the 852 nm laser, L-JA solutions can reach temperatures in excess of 60° C. rapidly and repeatedly (FIG. 2C, FIG. 2E). A lower concentration allows for a lower overall initial dose administered and extends the therapeutic window in which photothermal therapy is effective. Peak absorptions at wavelengths less than 890 nm indicate incomplete aggregation, such as in the optical absorbance spectrum of DSPC. Similar data is observed when DSPC particles are heated at the optimal temperature (60 to 65° C.) for the 16+ hours required to complete aggregation, which may be caused by complete lipid breakdown which obscures IcG in solution (FIG. 3A, FIG. 3B, FIG. 3C). Second, as wavelengths increase from 850 nm to about 1100 nm, there is reduced laser attenuation in biological tissues. Over this wavelength range, there is very little absorption from endogenous agents allowing laser signals to penetrate deeper into tissue and consequently effective illumination of deeper tumors. Third, as there is lower attenuation from endogenous agents and a lower concentration of particles is equally effective, it is possible to use lower power lasers. When there is high attenuation or low signal attenuation from administered contrast agents, a higher laser power is necessary to achieve the same effect. High power lasers are typically more expensive and less safe for the patient. Thus, the liposomal IcG J-aggregates can serve as the basis for a safer and more affordable system for photothermal therapy.

Furthermore, systemically administered IcG is cleared from circulation within an hour whereas liposomal IcG J-aggregates can circulate for hours. Liposomal IcG J-aggregates are more photostable than IcG and thus can be illuminated multiple times without a change in absorption or heating. Liposomal IcG J-aggregates are biodegradable unlike gold nanoparticles. Thus, systemically administered liposomal IcG J-aggregates can be cleared through natural biological processes whereas gold nanoparticles may remain in the body indefinitely. Additionally, the synthesis of liposomal IcG J-aggregates requires less resources and time than gold nanoparticles.

### Example 2: Optimization of the Synthesis of Liposomal J-Aggregates of IcG

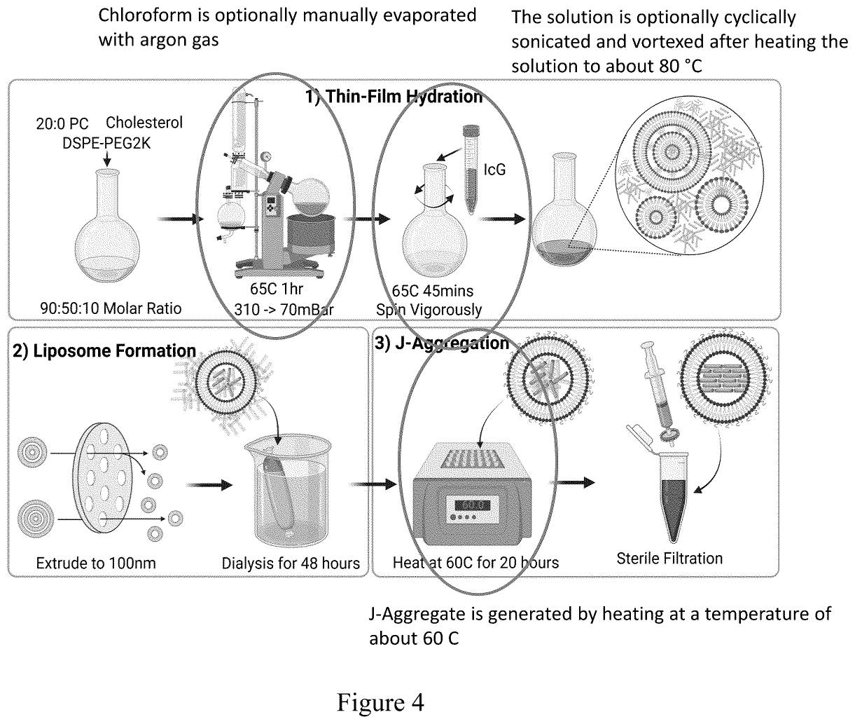

The synthesis of liposomal J-Aggregates is performed in three distinct steps, each of which were empirically adjusted (FIG. 4).

The ratio of PC to cholesterol to PEG underwent a series of trial and error where it was found that the optimal experimental ratio of PC:cholesterol:PEG was 85:50:15. Cholesterol assists with fluidity and heat response of the liposomes and it was determined that a fairly high amount was needed to create a stable liposome under high heat conditions. PEG adjusts the overall surface potential of the particle and improves circulation time and thus a higher amount than previously used was needed.

An additional step in the thin-film hydration stage was developed in which the lipids are manually dried in a small glass vial using a stream of argon gas. Manual drying followed by rotary evaporation allowed for more consistent and viable thin films of lipids which were not accessible using rotary evaporation, though in larger, more commercially viable batches, use of a rotary evaporator would be more practical. Drying also occurs at room temperature, instead of at 65° C.

Liposome formation was further optimized from the original procedure which involved heating at 65° C. and spinning for 45 minutes. The lipid films were hydrated with IcG, briefly heated to 80° C., then cyclically sonicated and vortexed. It was found that heating above the transition temperature was essential for film hydration and particle formation and limits the amount of time the lipids are under high heat. The sonication and vortex also assisted in dislodging any lipids stuck to the glass vial.

Lastly, it was found that maintaining the temperature of the stir plate at 60° C. was essential in synthetic optimization. Temperatures below 60° C. were not sufficient to induce aggregation while temperatures above 60° C. are unstable. The transition temperature of 20:0 PC is about 66° C. which therefore makes it important to stay below 66° C. during heating to prevent leakage of IcG. The heating step should occur in the dark and covered without outside light.

### Example 3: Characterization of Liposomal J-Aggregates

The key aspect of this L-JA synthesis method is the use of high transition temperature lipids (20:0 PC) as the bulk of the liposomal shell. High transition temperature lipids are essential for maintaining particle stability at elevated temperatures (up to 60° C.) during extrusion and the J-aggregation process. Substituting for more conventional lower transition temperature lipids increases the risk of IcG leakage, incomplete aggregation during extrusion and total liposomal collapse during the day-long heating. With no apparent change to biocompatibility and stability, and only minor adjustments to synthesis procedures, higher transition temperature lipids are ideally suited for this process and are the primary reason L-JA can consistently be produced in as little as 3 days.

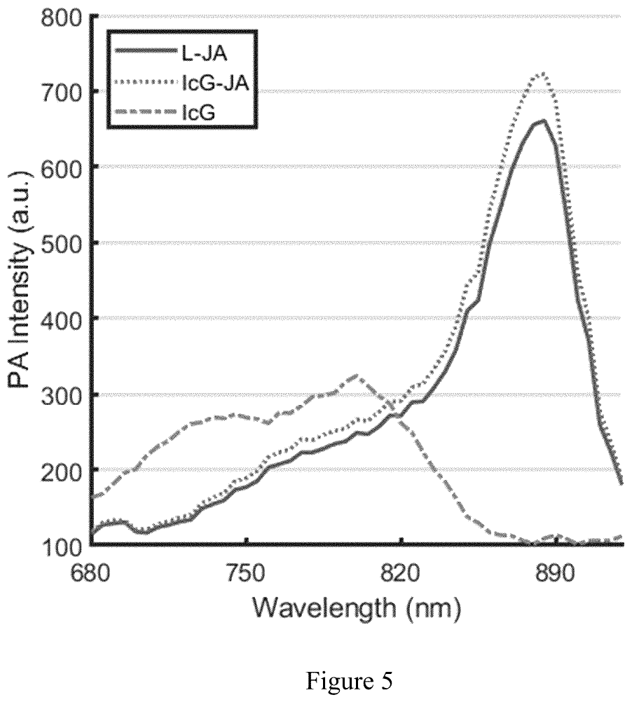

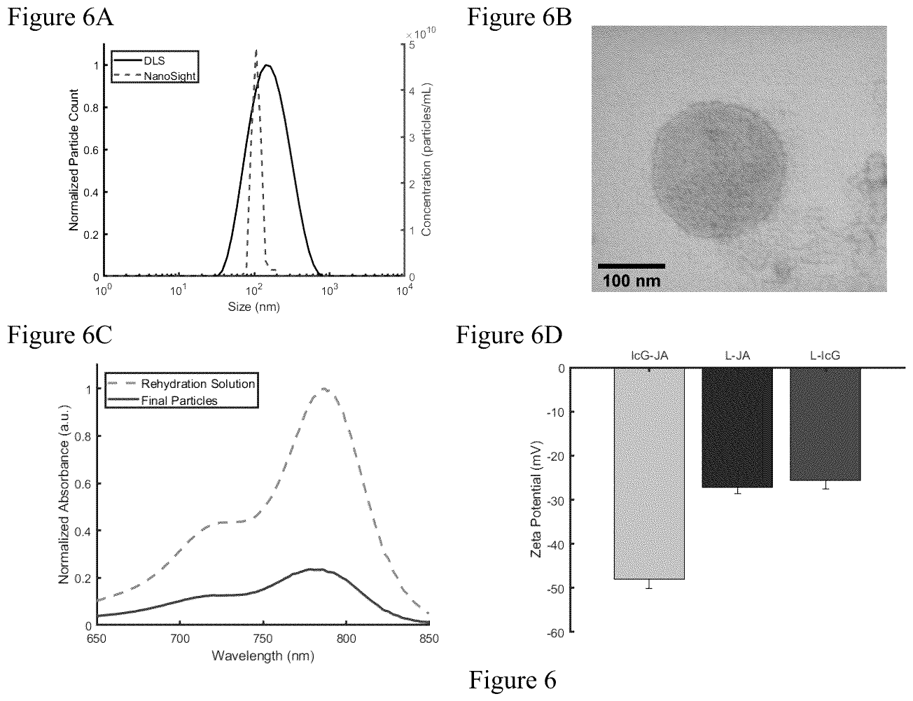

L-JA particles were analyzed to determine their physical and photoacoustic properties (FIG. 5). L-JA particles were characterized using dynamic light scattering (DLS), NanoSight, and Scanning Electron Microscopy (SEM). Through DLS measurement average particle diameter was found to be 129.26±28.54 nm (FIG. 6A) which was further confirmed with NanoSight measurement and SEM imaging (FIG. 6A and FIG. 6B). A consistent, monodisperse size range in the 100 s of nanometers is ideal for a contrast agent designed for lengthy circulation in the blood stream and potential extravasation to tumor tissue (Farooq, A et al., 2022, Nanomaterials (Basel), 12:393; Xu, L et al., 2022, Advanced NanoBiomed Research 2, 2100109). The encapsulation efficiency of IcG was calculated to be 28.12±4.27%. The zeta potentials of L-JA, liposomal IcG (L-IcG), and IcG-JA are −27.12±1.56 mV, −25.58±1.94 mV, and −48.05±2.15 mV respectively (FIG. 6D). The similar zeta potential value for L-JA and L-IcG can be attributed to the similarity in their liposomal formulation and shows the high transition temperature liposomal shell can withstand the heat of the aggregation process. The liposomal formulation had a considerably more neutral potential than the unencapsulated IcG-JA. This decreased zeta potential can be attributed to successful encapsulation and PEG coating. Moving to a more neutral potential without dropping below a 10 mV magnitude will ideally prevent long-term aggregation and improve stability while maintaining low toxicity. L-JA surface potential was found to be −27.12±1.56 mV which was no different than liposomal IcG (L-IcG) at −25.58±1.94 mV. The liposomal formulation had a considerably more neutral potential than the unencapsulated IcG-JA at −48.05±2.15 mV (FIG. 6D). This decreased surface potential can be attributed to successful encapsulation and PEG coating. Moving to a more neutral potential without dropping below a 10 mV magnitude prevents long-term aggregation and improve stability (Heurtault, B et al., 2003, Biomaterials, 24:4283-4300) while maintaining low toxicity (Schwegmann, H et al., 2010, Journal of Colloid and Interface Science, 347:43-48).

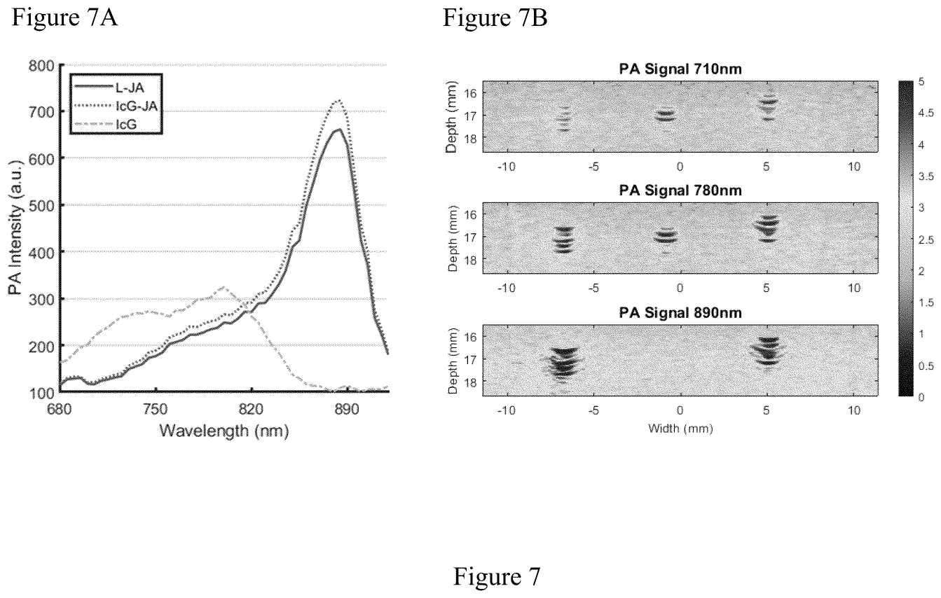

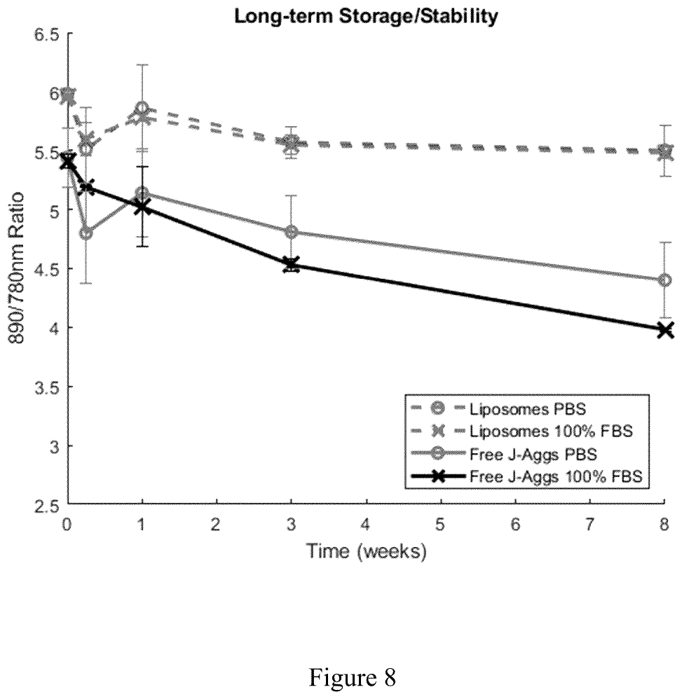

Following optimization of the physical properties of the particles, the photoacoustic properties of the L-JA particles were compared to unencapsulated and non-aggregated forms of IcG. Particles were measured in 100% FBS at 37° C. to more closely mimic the in vivo environment. At equal concentrations the L-JA peak signal intensity was nearly 3 times larger than that of IcG (FIG. 7A). Notably, the spectral profile of the encapsulated form, L-JA, was not different than that of unencapsulated IcG-JA, meaning the lipid coating did not interfere with laser absorption and signal generation. In tubes embedded within tissue-mimicking phantoms, a similar increase could be seen at peak wavelengths (FIG. 7B). Unlike IcG, which is known to rapidly clear due to binding with serum proteins and has different properties depending on concentration and solvent medium (Landsman, M. L. et al., 1976, J Appl Physiol, 40:575-583; Hironaka, K et al., 1973, Clinica Chimica Acta, 47:39-43), this relationship held true when the lipid particles were dispersed in water or a buffer solution like phosphate-buffered saline (PBS). Incubation in PBS, fetal bovine serum (FBS), and cell culture media at 4° C. and 37° C. showed that L-JA also have superior storage stability when compared to IcG. IcG is known to undergo degradation by serum proteins. IcG J-Aggregates will also experience some degradation when exposed to serum and the liposomal shell prevents degradation as the serum proteins are unable to bind to the IcG, degrading its signal (FIG. 8). PEGylation and overall lipid coating appeared to mitigate the spectral changes caused by media change and serum protein binding.

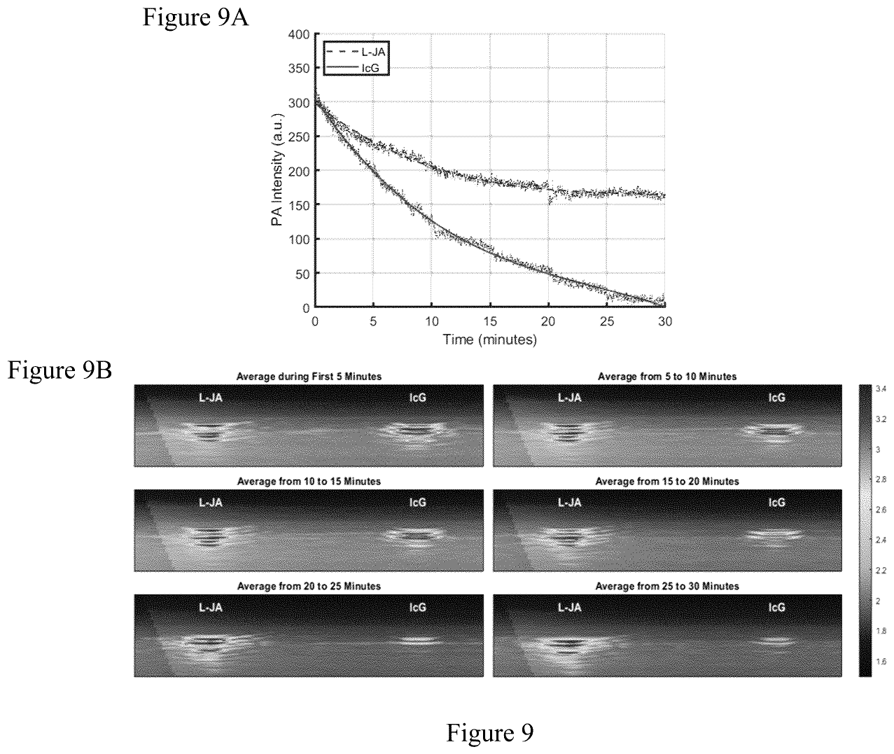

The intensity of IcG photoacoustic signal is known to degrade when irradiated with a pulsed laser repeatedly over time. This was confirmed through 30 minutes of imaging at 800 nm with just under 5 laser pulses per second (FIG. 9). IcG completely photodegraded during this time period with a signal drop of more than 50% between 5 and 10 minutes. L-JA is much more photostable than IcG. Under the same conditions, L-JA did not completely degrade and maintained a majority of its signal over the 30-minute imaging session. The same relationship was seen when imaging was performed at 890 nm. L-JA appeared to degrade slightly during initial imaging before maintaining a near constant signal with time. These results tracked well with other reports that J-aggregates of cyanine dyes appear to be more photostable than their non-aggregated forms (Cao, Y. M., 2018, Photostabilization of J-aggregate cyanine dyes for exciton-polariton based devices, Massachusetts Institute of Technology); Bricks, J. L. et al., 2017, Methods Appl Fluoresc, 6:012001). Higher photostability make these particles a much more attractive option for potentially lengthy and repeated imaging in a clinical environment.

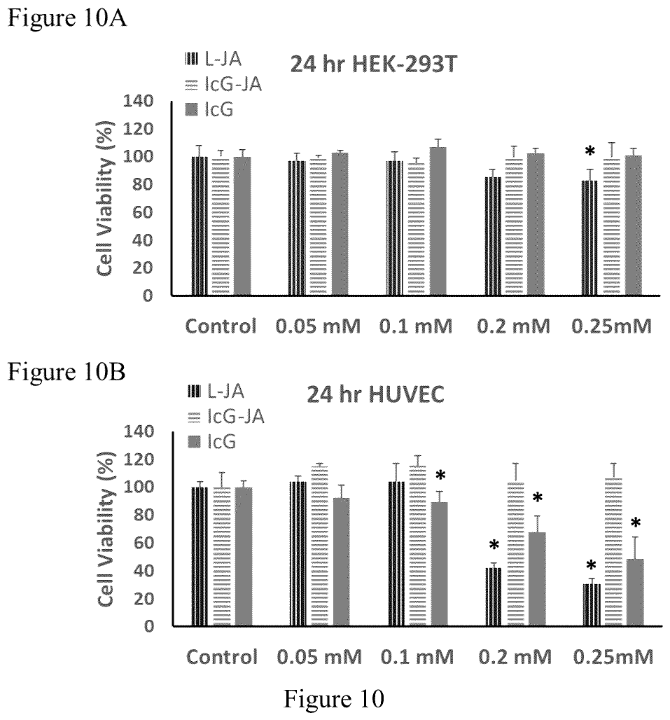

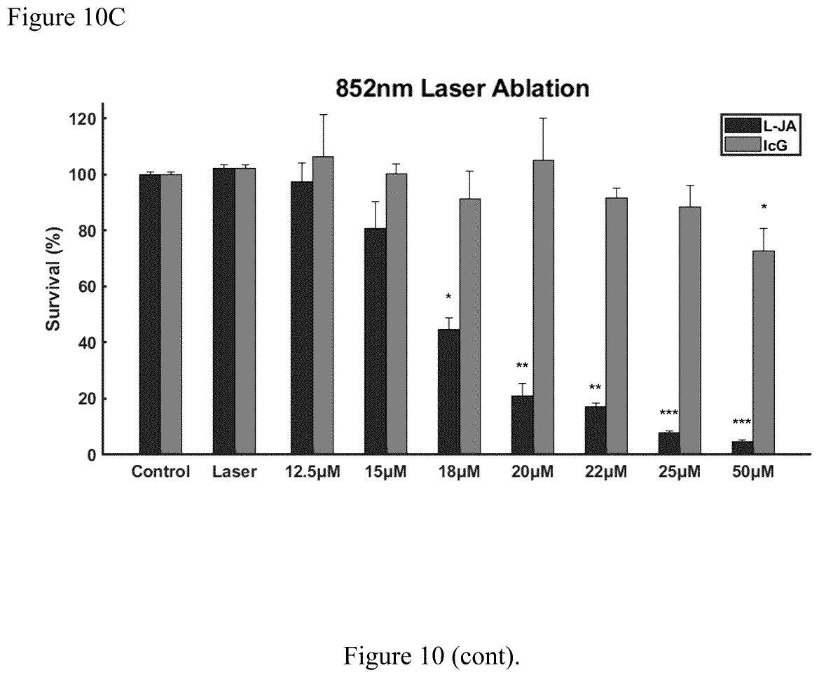

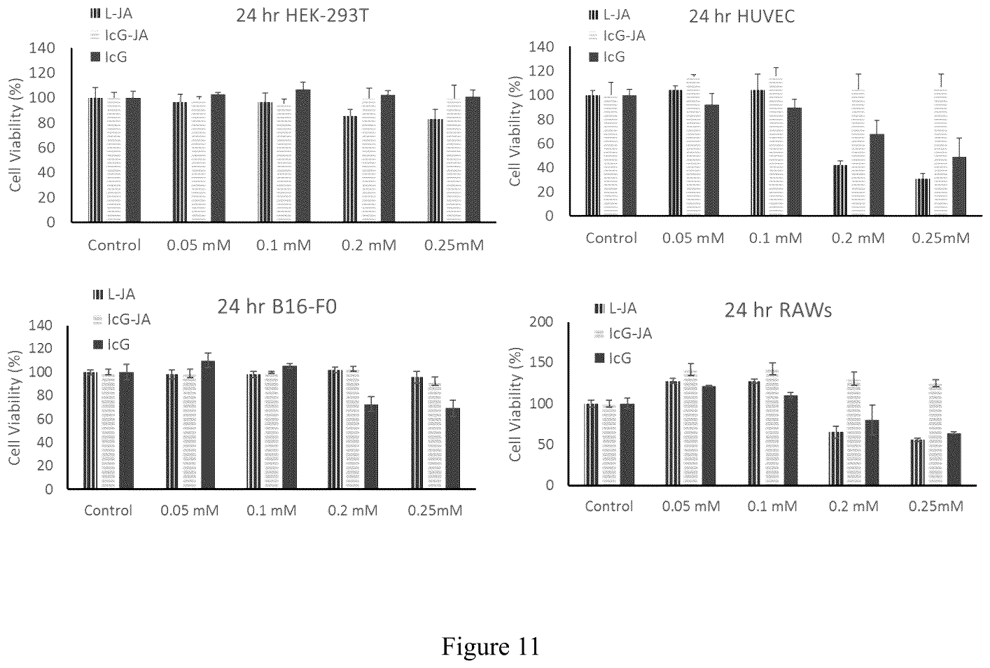

Before in vivo injections, potential toxicity was assessed with an MTS assay. When compared to control groups, minimal cytotoxicity was observed for L-JA, IcG-JA, and IcG in HUVECs and HEK-293T cell lines below 0.2 mM following 24 hours of incubation (FIG. 10 and FIG. 11). Similar results were observed after 48 hours and in B16-F0 and RAW cell lines. 0.2 mM is well above potential particle concentrations in vivo following injections, as a 5 mg/kg dose would be diluted far below this concentration. The consistency of these results across cell lines and time points supported the biocompatibility of both the FDA-approved IcG and the liposomal form presented here. This is a considerable advantage over other NIR exogenous agents that have mixed results for toxicity and overall biocompatibility (Xiong, P et al., 2022, Advanced Science, 9:2106049; Sani, A et al., 2021, Biochem Biophys Rep, 26:100991).

### Example 4: Photothermal Heating at 808 nm and 852 nm

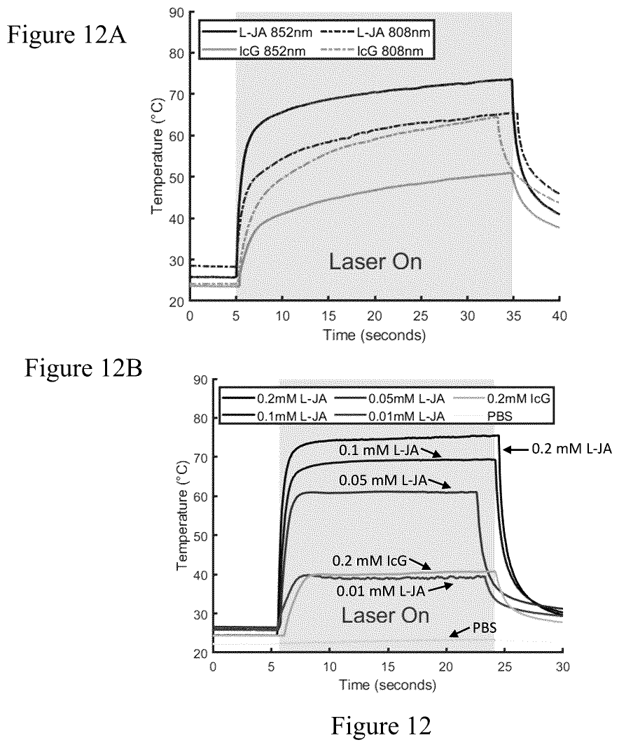

Beyond use as a contrast agent for imaging, the unique characteristics of IcG and IcG-JA have made them a promising candidate for PTT. Typically, 808 nm lasers are employed due to low cost and widespread availability. As shown previously, 808 nm lasers are very effective for IcG as this wavelength is very near the maximum absorbance of IcG in plasma. IcG-JA and L-JA also perform well at 808 nm, showing a similar amount of heating as IcG. Additionally, the same improvements seen for photostability in imaging carry over to PTT applications. Maintaining the ability to respond favorably to laser illumination following multiple exposures makes IcG-JA and L-JA much more attractive for therapeutic applications where cyclic or lengthy heating would likely be required. While IcG-JA serve as good heat sources when illuminated at 808 nm, the redshifted peak absorbance of IcG-JA and L-JA suggest that they would perform even better at longer wavelengths. For the first time, that can be seen here with the use of an 852 nm laser (FIG. 12). While solutions of IcG and L-JA reach similar temperatures when illuminated at 1 W/cm² at 808 nm, L-JA reach a much higher temperature (FIG. 12A) compared to IcG when illuminated with 0.4 W/cm² at 852 nm. With the ability to reach temperatures in excess of 70° C., the performance of L-JA at 852 nm is nearly 50% more effective than IcG at 852 nm and is nearly 25% more effective than both L-JA and IcG at 808 nm. Heating of L-JA at 852 nm is considerably more efficient as a solution of L-JA reaches the same temperature as an IcG solution at a 20× higher concentration (FIG. 12B). Coupled with potentially deeper penetration of light, heating at 852 nm is more efficient for L-JA so it is likely that fewer particles would need to reach the target site or lower laser powers would be needed to achieve the same level of therapeutic effect. These improvements suggest a longer wavelength closer to the 890 nm peak absorbance of L-JA could be even more effective. As an organic agent, L-JA offer a simple, biocompatible, biodegradable and photostable alternative to the metallic and inorganic particles previously necessary for PAI and PTT between 850 and 1000 nm.

### Example 5: In Vivo Characterization of Liposomal J-Aggregates as Contrast Agents

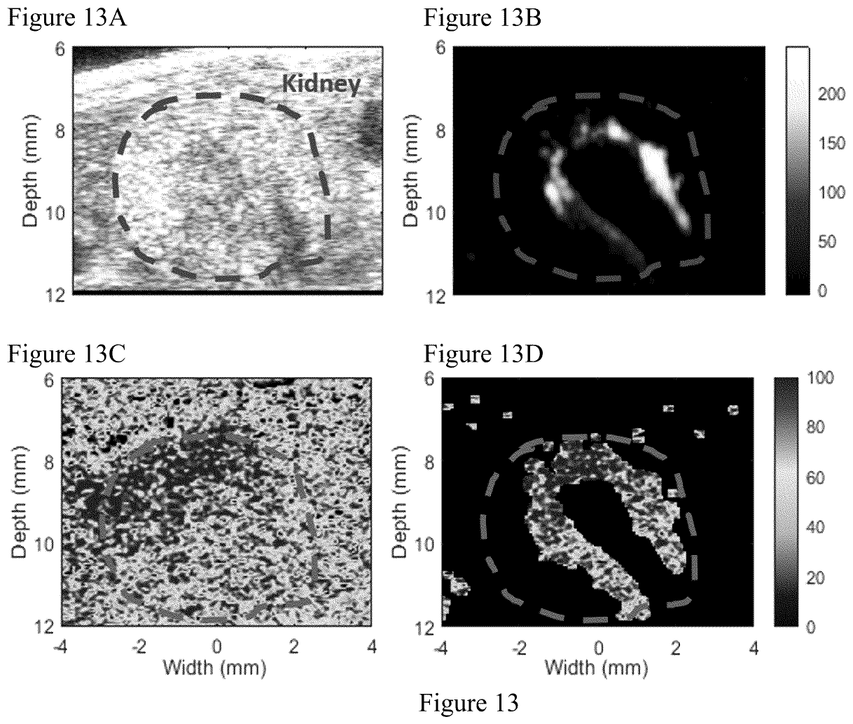

Particle circulation was monitored by imaging the mouse kidney and surrounding tissue. The kidney is a high blood flow organ that has the distinct advantage of being easily and rapidly identifiable using ultrasound imaging. Simple setup and identification decreases the amount of time animals are under anesthesia and limits any potential artificial lowering of SO₂ due to prolonged exposure to anesthetic agents. Once prepped, mice placed in the imaging system were imaged with power-doppler to confirm blood flow and baseline values for SO₂ and contrast agent signals (FIG. 13). Following a bolus injection of a 5 mg/kg dose of contrast agent via the tail vein, the same region was monitored at varying time points. After processing with FUJIFILM VisualSonics' proprietary spectral unmixing software the images were analyzed using in-house MATLAB software. Power-doppler images guide the placement of ROIs around areas of high blood flow allowing isolation and calculation of SO₂ and contrast agent signal over the course of imaging.

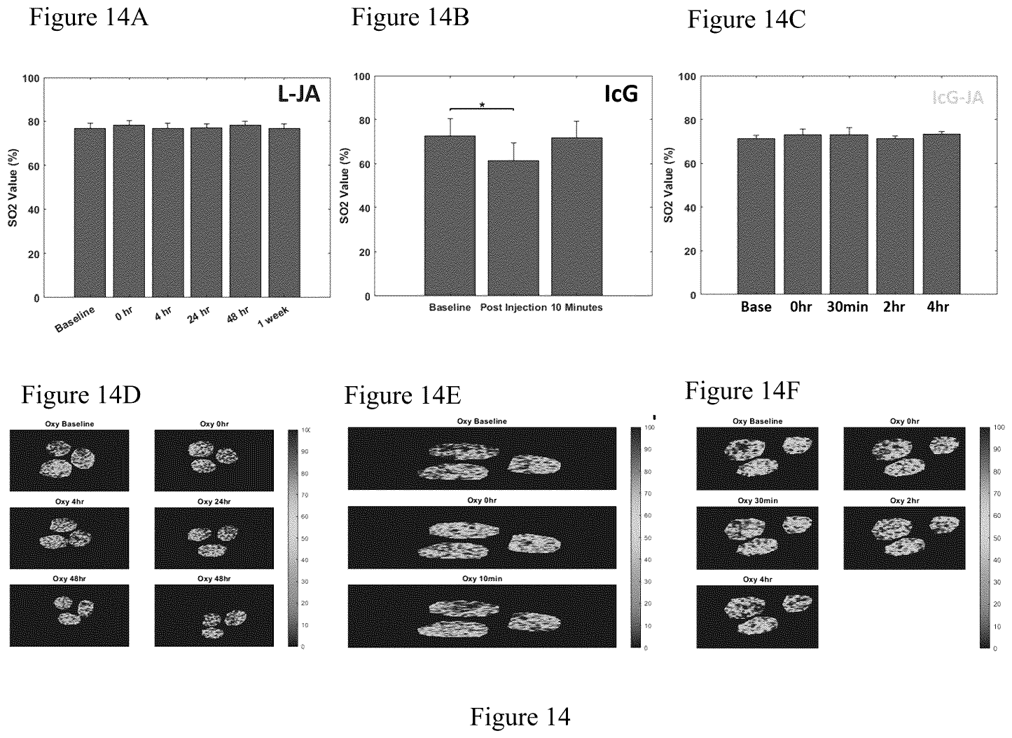

For all injections, “0 hr” refers to data collected immediately after bolus injection of contrast agent. Neither L-JA or IcG-JA caused a significant change in measured SO₂ levels (FIG. 14). Even at its highest concentration immediately following injection, the variation in SO₂ value was minimal to none. IcG, however, caused a significant drop in measured SO₂ levels through the duration of its brief circulation time in the body. During imaging, SO₂ varied by less than 2 to 3% in mice receiving L-JA and IcG-JA while SO₂ dropped by as much as 15 to 20% in mice receiving IcG. IcG inhibited accurate SO₂ determination while J-aggregated forms of IcG did not. This characteristic of L-JA is very important, particularly in functional PAI, as it enables more reliable oxygen saturation measurement in the presence of an exogenous contrast agent. L-JA therefore may have significant potential as a diagnostic and functional PA contrast agent.

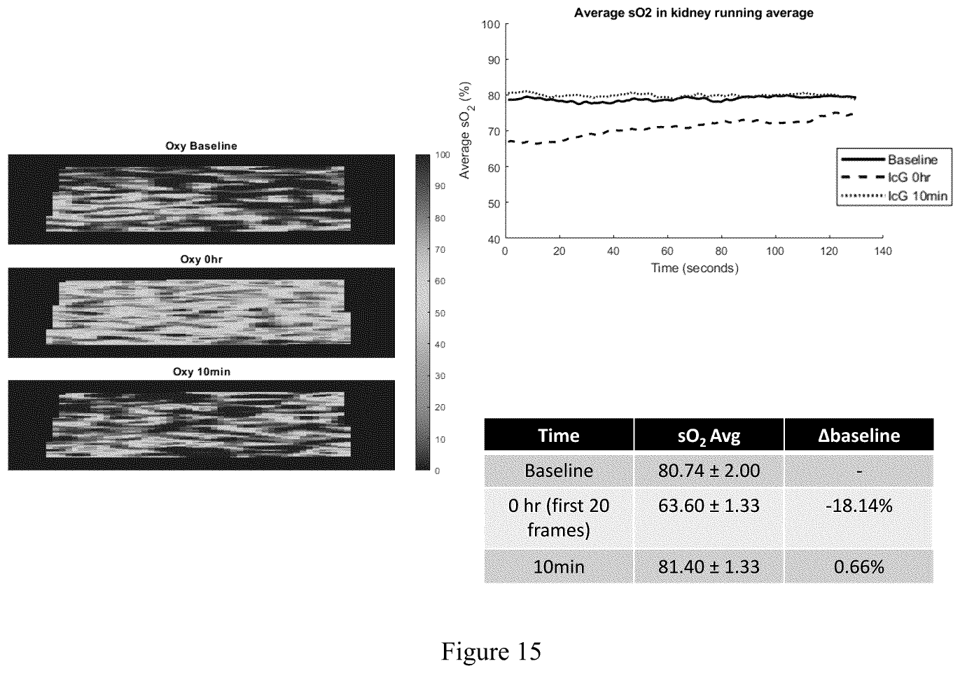

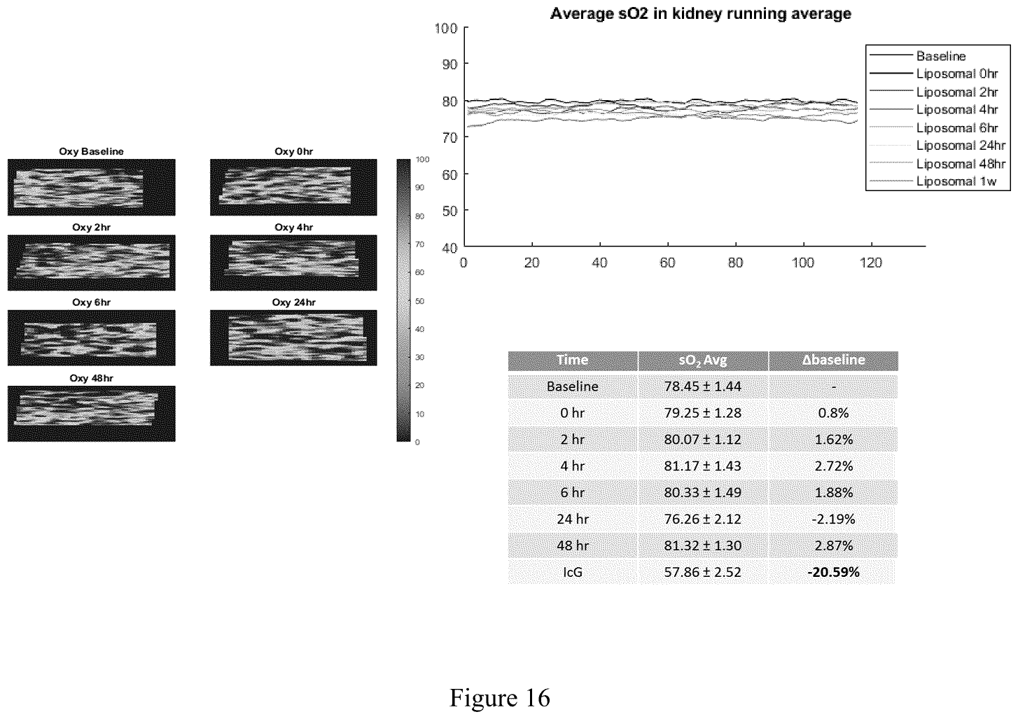

From injection to full clearance, 1 week for L-JA and 1 hour for IcG-JA, J-aggregate particles did not cause a significant change in measured SO₂ levels. Even at its highest concentration immediately following injection, SO₂ showed no discernible change from baseline. IcG, however, caused a significant drop in measured SO₂ levels through the duration of its brief circulation time in the body. IcG inhibited accurate SO₂ determination while J-aggregated forms of IcG did not. A large drop in SO₂ occurs following injection and a slow climb back to baseline level occurs as IcG clears, which does not occur with IcG-JA or L-JA (FIG. 15 and FIG. 16).

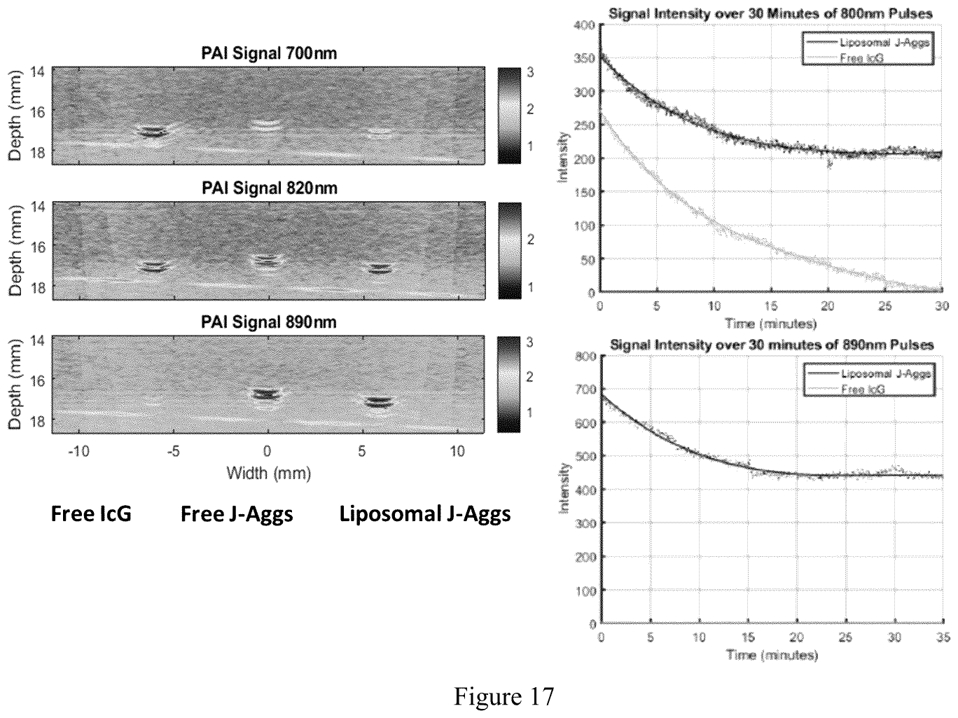

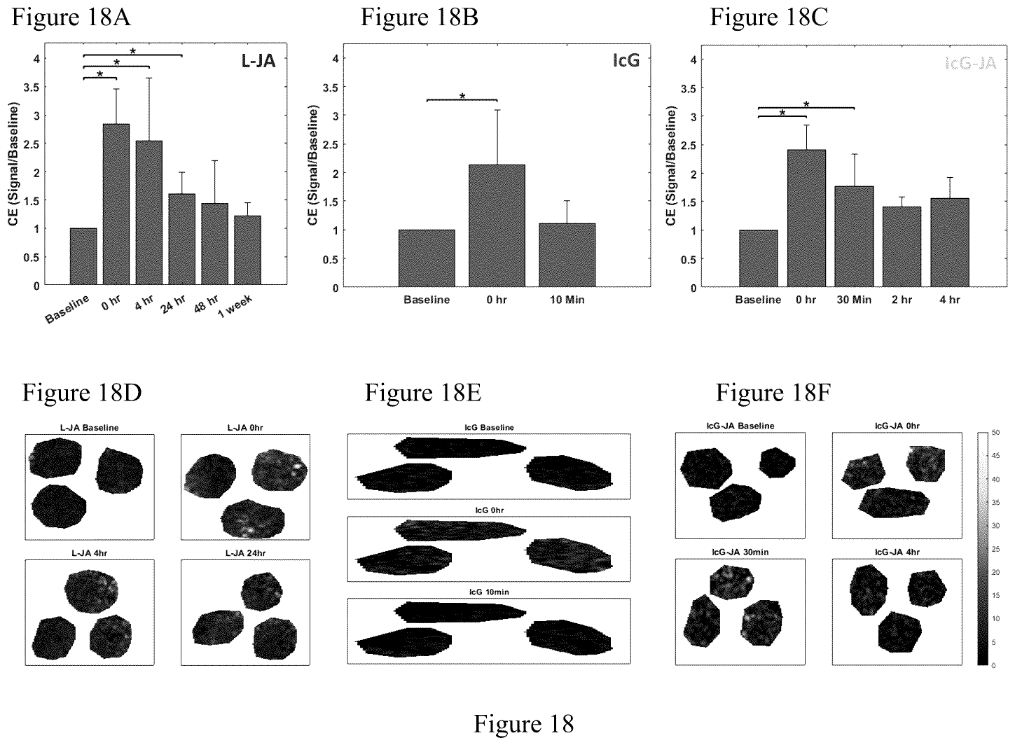

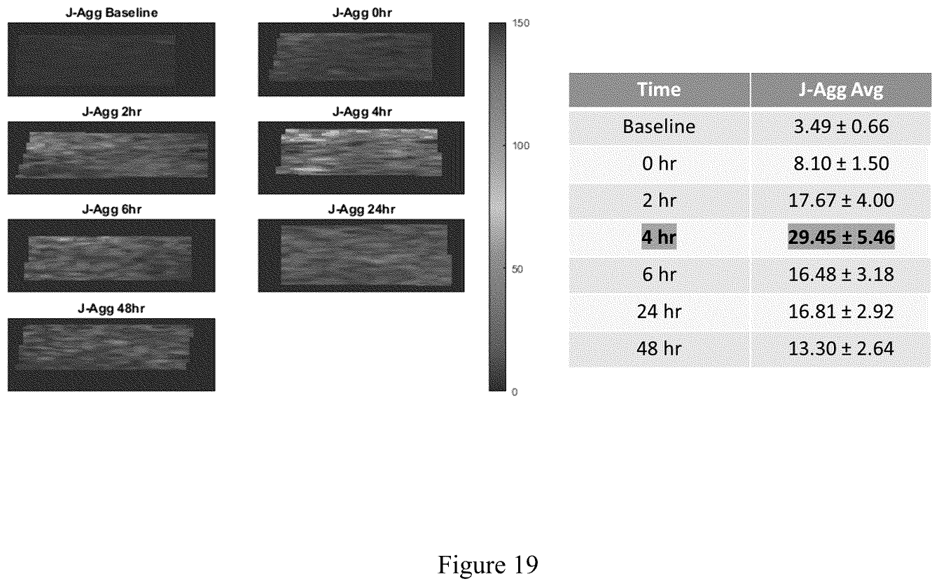

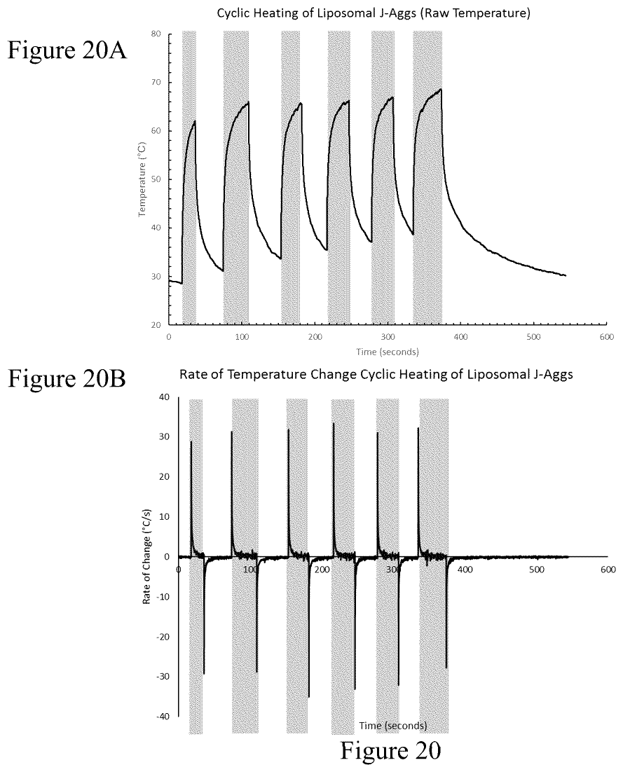

In vivo contrast enhancement (signal at time point/baseline signal) from the exogenous agents closely followed in vitro characteristics (FIG. 17). L-JA provided a significant contrast enhancement for up to 24 hours following injection (FIG. 18A, FIG. 18B, and FIG. 18C). Unmixed L-JA signal was more than 2.5 times the baseline up to 4 hours after injection. IcG-JA also provided significant enhancement. IcG provided a significant contrast enhancement, however this was at the cost of blocking sO₂ determination and was limited to the very brief circulation time of the molecule. In addition, the increased contrast it provided was considerably lower than the signal enhancement provided by an equal concentration of J-aggregate particles (FIG. 18D, FIG. 18E, FIG. 18F, FIG. 19, and FIG. 20). Interestingly, IcG-JA circulated significantly longer than the non-aggregated form but not nearly as long as its encapsulated form. Moreover, cyclic heating of liposomal J-Aggregates using a 808 nm laser resulted in temperature changes of liposomal J-Aggregates during cyclic heating. Cyclic heating of liposomal J-Aggregates did not degrade the liposomal J-Aggregates highlighting their utility in phototherapy (FIG. 20).

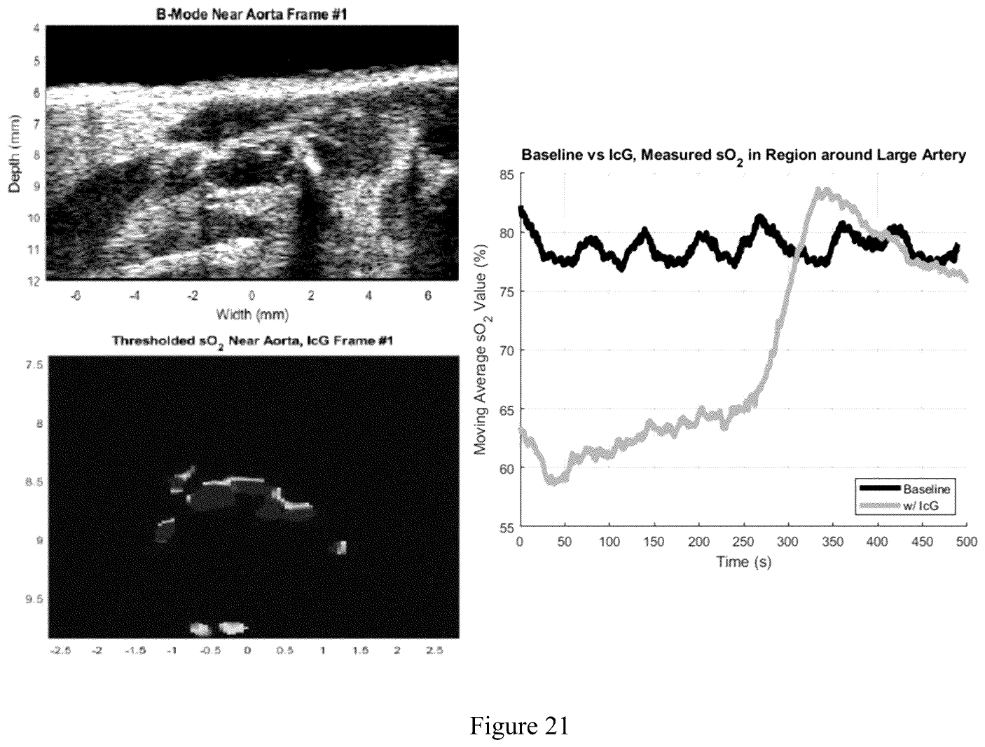

In agreement with other groups that have successfully worked with IcG-JA and its liposomal encapsulations (Liu, R et al., 2017, Nanotheranostics, 1:430-439; Wood, C. A. et al., 2021, Nat Commun, 12:5410; Cheung, C. C. L. et al., 2020, Nanotheranostics, 4:91-106), these particles offer distinct advantages over monomeric IcG and other NIR contrast agents for photoacoustic imaging (FIG. 21). Non-encapsulated IcG-JA offer similar improvements in terms of signal generation and sO₂ determination, but L-JA are superior in terms of their homogeneity, longer circulation time, and versatility. When not encapsulated, IcG-JA grow to a wide range of sizes with many as large as several hundred nanometers in diameter. For safe injection and consistent imaging, a vast majority of these particles needed to be filtered out through a tedious process that wastes IcG precursor. The presented procedure for liposomal encapsulation provides a dramatic improvement over other methods due to its increased speed, overall reproducibility, and potential scalability, while maintaining distinct advantages in peak wavelength, signal intensity, and circulation time. The high-transition temperature lipids used in this procedure allow for heating and aggregation of pre-encapsulated IcG which dramatically decreases aggregation time in comparison to the weeks-long process of aggregation at room or refrigerated temperatures. Heating the IcG at optimal temperatures also alleviates potential inconsistencies that result from self-initiated-aggregation or through extrusion alone. While it appears extrusion can initiate the aggregation process, especially at high temperatures, it is inconsistent in its final results and requires supplementary heating to ensure complete aggregation. Using conventional liposome formulation methods also makes this process easily scalable. With large rotary evaporators and thermal-barrel pressurized extruders, the process can be upscaled and almost entirely automated.

### Example 6: Oxygen Saturation Measurement In Vivo

To assess their performance in vivo, particles were injected intravenously via the tail vein. Particle circulation was monitored by imaging the mouse kidney and surrounding tissue. The kidney is a highly vascularized organ that has the distinct advantage of being easily identifiable using ultrasound imaging. Simple setup and identification decrease the amount of time animals are under anesthesia and limit any potential artificial lowering of SO₂ due to prolonged exposure to anesthesia. Mice positioned in the multimodality imaging system had kidneys located and imaged first with Power Doppler to confirm blood flow. The photoacoustic modality was then used to measure and establish a baseline value for SO₂ before injecting contrast agents. Following a bolus injection of contrast agent via the tail vein, the same region was monitored at multiple time points. The PA images were spectrally unmixed followed by image analysis using in-house MATLAB software. Power Doppler images were used as a guide for selecting ROIs around areas of high blood flow allowing isolation and calculation of SO₂ and contrast agent signal.

For all injections, “0 hr” refers to data collected immediately after bolus injection of contrast agent. Neither L-JA nor IcG-JA caused a significant change in measured SO₂ levels. The variation in SO₂ value was negligible even at the highest concentration of each agent. IcG, however, caused a significant drop in measured SO₂ levels through the duration of its brief circulation time in the body. During imaging, SO₂ varied by less than 2 to 3% in mice receiving L-JA and IcG-JA while sO₂ dropped by as much as 15 to 20% in mice receiving IcG. IcG in the blood impaired accurate sO₂ determination while J-aggregated forms of IcG had a negligible effect. This attribute of L-JA is very important, particularly in functional PAI, as it enables reliable measurement of blood oxygen saturation in the presence of an exogenous contrast agent. Therefore, L-JA may have significant potential as a biocompatible diagnostic, functional, and therapeutic PA contrast agent.

### Example 7: Contrast Enhancement and Circulation In Vivo

In vivo contrast enhancement (signal at time point/baseline signal) from the exogenous agents closely follows in vitro characteristics. L-JA provides a significant contrast enhancement for up to 24 hours following injection (FIG. 18). Unmixed L-JA signal is more than 2.5 times the baseline up to 4 hours after injection. IcG-JA also provides significant enhancement for up to 30 minutes after injection, but like IcG it is also rapidly cleared. As expected IcG provides a significant contrast enhancement; however, this comes at the cost of blocking sO₂ measurement and is limited to the very brief circulation time of the fluorophore (FIG. 21). In addition, the increased contrast provided by IcG is considerably lower than the signal enhancement provided by an equal concentration of J-aggregate particles (FIG. 18). Interestingly, IcG-JA circulate significantly longer than the non-aggregated form but not nearly as long as its encapsulated form.

In agreement with other groups that have successfully worked with IcG-JA and its liposomal encapsulations, these particles offer distinct advantages over IcG and other NIR contrast agents for photoacoustic imaging. Non-encapsulated IcG-JA offer similar improvements in terms of signal generation and sO₂ determination, but L-JA are superior in terms of their homogeneity, longer circulation time, and versatility. When not encapsulated, during the J-aggregation process IcG-JA can grow to a wide range of sizes, varying from several hundred nanometers to microns in average diameter. Moreover, unencapsulated IcG-JA can have a strong negative surface charge which may lead to increased macrophage uptake and faster clearance from circulation (Duan, X., 2013, Small, 9:1521). For safe injection and consistent imaging, a vast majority of these particles need to be filtered out through a tedious process that reduces yield. The procedure for liposomal encapsulation reported in this article provides a dramatic improvement over other methods due to its increased speed, overall reproducibility, and potential scalability, while maintaining distinct advantages in peak absorption wavelength, photoacoustic signal intensity, and circulation time. The high-transition temperature lipids used in this procedure allowed for heating and aggregation of pre-encapsulated IcG, which dramatically decreased aggregation time from weeks to a few days. Heating the IcG at this optimal temperature also alleviated potential inconsistencies that result from self-initiated-aggregation or through extrusion alone. While it has been reported that extrusion can initiate the aggregation process, especially at high temperatures, it is inconsistent in its final results and requires supplementary heating to ensure complete aggregation. Using conventional liposome formulation methods further makes this process easily scalable. With large rotary evaporators and thermal-barrel pressurized extruders, the process can be upscaled and potentially automated.

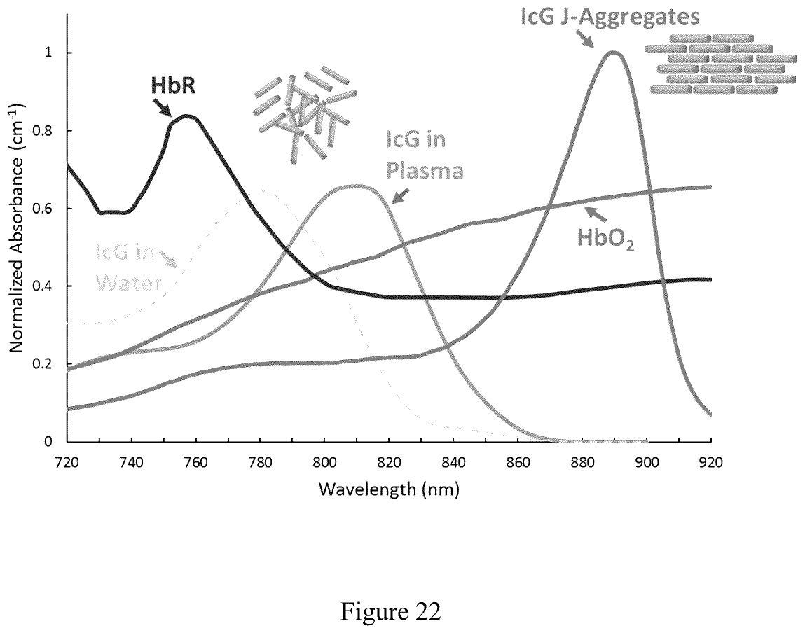

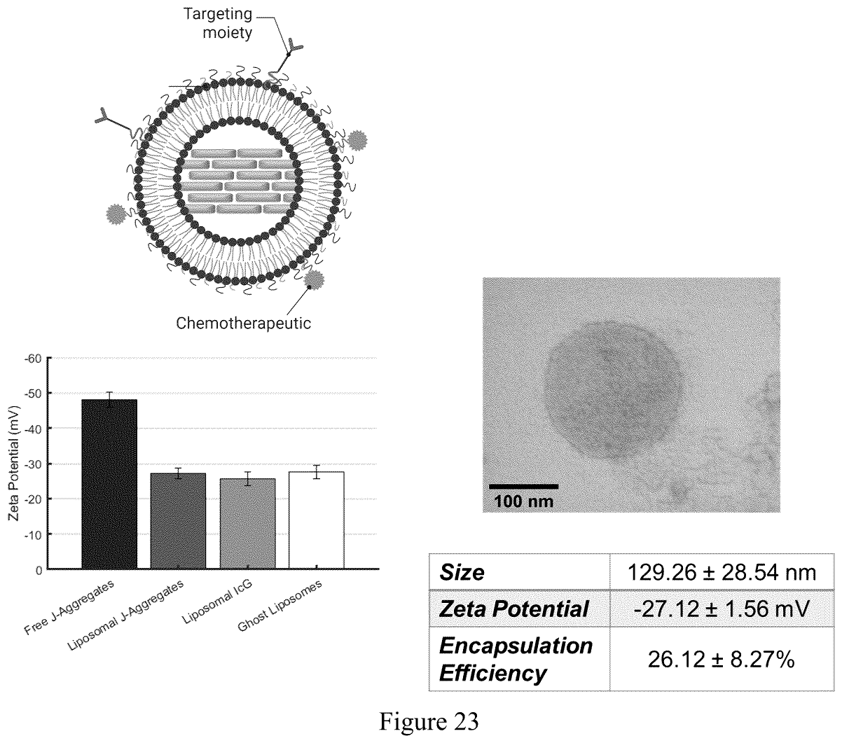

The present invention relates to, in part, a novel procedure for developing Liposomal J-Aggregates of IcG and a novel methodology for performing photothermal therapy with laser wavelengths higher than 850 nm. IcG, a widely available and federally approved near-infrared contrast agent, has profound photoacoustic and photothermal characteristics. When an IcG solution is prepared and heated using the method disclosed herein, the particles begin to form aggregates called J-Aggregates, which offer improved optical properties, namely a significant redshift deeper into the near-infrared range and an increase in peak signal magnitude at 890 nm (FIG. 22). Liposomes are lipid-based nanoparticles that are highly modifiable and confer various advantages for particle delivery. The use of liposomes allows for control of particle size, monodispersity, prolonged circulation time with PEGylation, the potential for adding targeting motifs, and co-treatments with therapeutic agents. Incorporating IcG J-Aggregates inside liposomes limits their size to hundreds of micrometers, dramatically improving their circulation time and increasing the overall delivery of these particles to anatomical areas of interest (FIG. 6, FIG. 23).

The presented procedure for liposomal encapsulation provides a dramatic improvement over other methods due to its increased speed, overall reproducibility, and potential scalability, while maintaining the distinct advantages in peak wavelength, signal intensity, and circulation time. Using high-transition temperature lipids allows for heating and aggregation of already encapsulated IcG which dramatically decreases aggregation time in comparison to the weeks long process of aggregation at room or refrigerated temperatures. Heating the IcG at optimal temperatures also alleviates potential inconsistencies in aggregation that can occur when relying on extrusion alone. Using conventional liposome formulation methods also makes this process easily scalable. With larger scale rotary evaporators and thermal-barrel pressurized extruders, the process can also be almost entirely automated.

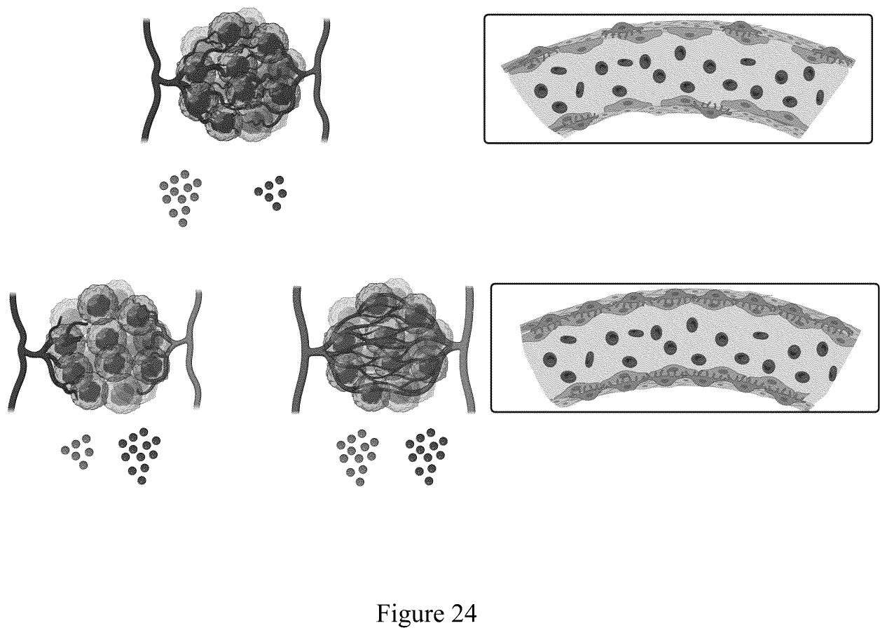

Vascular normalization is a promising avenue for cancer therapy. Tumor vasculature is characterized by immature, tortuous vessels and depleted and ineffective endothelial cells and pericytes, and overall has imbalanced angiogenic factors. Conversely, normalized vasculature is characterized by mature, complete vessels and restored endothelial cell and pericyte coverage, and overall has balanced angiogenic factors. When a balance of pro- and anti-angiogenic factors is maintained, small leaky vessels may be pruned allowing for improved delivery of oxygen and potentially therapeutics. Finding this normalization “therapeutic dose” is important as too low a dose will be ineffective and too high a does may prune vasculature too much leading to a more hypoxic and aggressive tumor (FIG. 24).

L-JA offer a unique combination of biocompatibility, biodegradability, photostability, and photoacoustic signal generation. With one injection in vivo, they are a multimodal contrast agent allowing for diagnostic, functional, and therapeutic applications. Herein, we have detailed a process that consistently produces these L-JA at higher yields in a considerably shorter time period than previously reported. Reliant on the use of higher transition temperature lipids, IcG-JA are formed within fully formed liposomes. Using lipids with a higher gel-to-liquid expanded phase transition temperature allows for rapid heat-induced aggregation of IcG and reduces potential leakage from the liposome during heating. Once formed, L-JA are not only safe at physiologically relevant concentrations, but are brighter and more photostable than IcG. Unlike IcG, the substantial redshift in peak signal generation directly resulting from J-aggregation allows for more accurate real-time SO₂ estimation while L-JA are circulating. In addition, the PEGylated-liposomal shell ensures a monodisperse and regulated distribution of particles that will circulate significantly longer than unencapsulated IcG and IcG-JA. L-JA also show robust photothermal effects, and as previously reported may be a more viable candidate for therapeutic applications. As shown here for the first time, L-JA respond well to laser wavelengths closer to its peak absorbance. Here an 852 nm laser was significantly more effective than the conventionally used 808 nm laser, and suggests an 890 nm laser would be optimal. Furthermore, L-JA are somewhat unique as an organic agent that responds to longer wavelengths for both imaging and therapy without the need for complex metallic nanoparticles. Utilizing biocompatible and biodegradable materials in this manner may hasten the clinical translation of L-JA for PAI and PTT.

This facile approach to L-JA formations lends itself well to being a viable starting point for further specialization and functionalization. As they are, these particles could easily be utilized as contrast agents for photothermal therapy. If desired, a targeting motif could be readily added via slight adjustment to PEG content or inclusion of an active targeting agent. Or, as liposomes are commonly used as a drug delivery vessel, a drug could easily be conjugated to the liposome surface (Shin, D. H. et al., 2015, J Biomed Nanotechnol, 11:1989-2002; Kuesters, G. M. et al., 2010, Nanomedicine (Lond) 5:181-192) or included via passive or active loading (Alyane, M et al., 2016, Saudi Pharm J, 24:165-175; Wehbe, M et al., 2017, Journal of Controlled Release, 252:50-61). Together, this robust protocol allows for the rapid and reproducible formulation of a powerful clinically viable photoacoustic contrast agent with both diagnostic and therapeutic potential.

The materials and methods employed in the present experimental examples are now described.

### Materials

Indocyanine Green (IcG) was purchased from Chem-Impex (Wood Dale, IL). 1,2-diarachidoyl-sn-glycero-3-phosphocholine (20:0 PC), 1,2-distearoyl-sn-glycero-3-phosphoethanolamine-N-[methoxy(polyethylene glycol)-2000] (DSPE-PEG2000), and cholesterol were purchased from Avanti Polar Lipids (Birmingham, AL). Chloroform, ethanol, ammonium persulfate (APS), N,N,N′,N′-Tetramethylethylenediamine (TEMED), and titanium oxide (TiO₂), were purchased from Sigma-Aldrich (St. Louis, MO). 40% 19:1 acrylamide/bisacrylamide was obtained from Research Products International (Mt. Prospect, IL). Black India ink was purchased from Chartpak (Leeds, MA). MTS Proliferation Assay was purchased from Promega (Madison, WI). Copper grids and uranyl acetate solution were purchased from Electron Microscopy Sciences (Hatfield, PA).

### IcG Calibration Curve

Indocyanine Green was dissolved in a 1:1 (v/v) solution of ethanol and ultra-pure deionized water to achieve an initial concentration of 10 mM. Solutions were vortexed and allowed to rest for 30 minutes to ensure that all indocyanine green powder was fully dissolved. The initial solution was serially diluted 9 times down to a concentration of 0.02 mM. Using a NanoDrop Spectrophotometer (Thermo Scientific, Waltham, MA), UV-VIS spectra for each solution were collected. This was repeated a total of four times. Raw values for the 780 nm absorbance peak, specific to monomeric IcG, were averaged and used to generate plots of absorbance intensity versus concentration. A linear relationship was observed up to a concentration of 2 mM, allowing for a simple curve to be generated for this region. To measure IcG content, all particle solutions were combined in a 1:1 ratio with ethanol and compared to this curve.

### J-Aggregate Liposome Synthesis

20:0 PC, cholesterol, and DSPE-PEG2000 dissolved in chloroform were combined in a 6/3/1 molar ratio inside a 4 mL glass vial. Chloroform was evaporated under a steady stream of argon gas until a thin film developed at the bottom of the vial. The vial was then placed inside a larger glass vessel connected to a Rotavapor R-300 rotary evaporator (Buchi, Berlin, Germany). The vial was left in the rotary evaporator set to 80 mBar for at least 2 hours to ensure all solvent was removed. 2 mL of a 2.5 mM IcG solution in ultra-pure deionized water was added to the vial to rehydrate the lipid film. The solution is heated to 75° C. and cyclically vortexed and sonicated in a bath sonicator (Fischer Scientific, Hampton, NH) for about an hour to complete the formation of liposomal vesicles.

To reduce the particles to their final more uniform size, this solution was then passed through 100 nm pore size polycarbonate membranes (Avanti Polar Lipids) 11 times using an extruder (Avanti Polar Lipids) and two glass syringes (Hamilton, Reno, NV). To remove unencapsulated IcG, the liposome solution was then dialyzed against deionized water for 48 hours using 300 kDa dialysis tubing (Fischer Scientific). Water was replaced every 8 to 12 hours. J-Aggregation of encapsulated IcG was completed by heating the liposomal solution in a heating block (Fischer Scientific) set to 60° C. for 24 hours. To ensure aggregation had completed, the UV-VIS spectra of the solution was obtained using a UV-VIS-NIR Spectrophotometer (Shimadzu, Kyoto, Japan). Aggregation was considered successful if the 890 nm signal was at least 3 times the signal at 780 nm. Most formulations were found to have 890 nm signals as much as 5 times the 780 nm signal. After J-Aggregation the particles were concentrated via centrifugation with 100 kDA filter units (Sigma-Aldrich) and resuspended in 1× phosphate-buffered saline for storage at 4° C. Particles were used within 1 week of synthesis.

### Particle Characterization

Size and zeta potential were measured using a Zetasizer (Malvern Panalytical, Malvern, UK). Particles were diluted by 1000× in 1×PBS for measurement. Polystyrene cuvettes were used for size measurements whereas standard zeta cells were used for zeta potential measurements. Particle density was determined by Nanosight (Malvern Panalytical). Starting with a solution of nanoparticles with a known concentration of J-aggregates, particles were again diluted by 1000×. The initial known concentration of J-aggregates is combined with the measured particle concentration to back-calculate the number of particles associated with each treatment concentration. Particle stability measurements were performed using a BioTek (Winooski, VT, USA) plate reader. Particles were incubated with PBS, 10% FBS, cell culture media, and 100% FBS in covered well plates either at 4° C. or in an incubator at 37° C. STEM imaging was performed using the Hitachi S-5500 SEM/STEM (Ibaraki, Japan). Particles were diluted and then drop-casted on ozone-cleaned copper grids. Particles were then stained with a 2% uranyl acetate solution and imaged at 30 keV.

Particle concentration was determined using an IcG calibration curve. To determine a sample concentration, 20 μL of J-aggregate solution would be added to 20 μL of 100% ethanol for a 1:1 final concentration that follows the pre-generated calibration curve. Ethanol is used because it can permeabilize the liposomal shell and importantly breaks the J-aggregates back into monomeric IcG. UV-VIS spectra of these ethanol diluted samples are obtained and used to determine the concentration of the samples in terms of IcG. The 780 nm peak value is used to determine how much to dilute or concentrate the sample solution to achieve the desired concentration.

### DLS, Zeta Potential, NanoSight, Plate Reader Stability Measurements, STEM

Size and surface potential were measured using a Zetasizer (Malvern Panalytical, Malvern, UK). Particles were diluted by 1000× in 1×PBS inside polystyrene cuvettes for size measurements and inside standard zeta cells for surface potential measurements. Each sample was run three times. Particle density was determined with the use of a NanoSight (Malvern Panalytical). Starting with a solution of nanoparticles with a known concentration of J-aggregates, particles were again diluted by 1000×. The initial known concentration of J-aggregates is combined with the measured particle concentration to back calculate the number of particles associated with each treatment concentration. Particle stability measurements were performed using a plate reader from BioTek (Winooski, VT). Particles were incubated with PBS, 10% FBS, cell culture media, and 100% FBS in covered well-plates either at 4° C. or in an incubator at 37° C. STEM imaging was performed using the Hitachi S-5500 SEM/STEM (Ibaraki, Japan). Particles were diluted then drop-casted on ozone-cleaned copper grids. Particles were then stained with a 2% uranyl acetate solution and imaged at 30 keV.

### In Vitro Photoacoustic Imaging

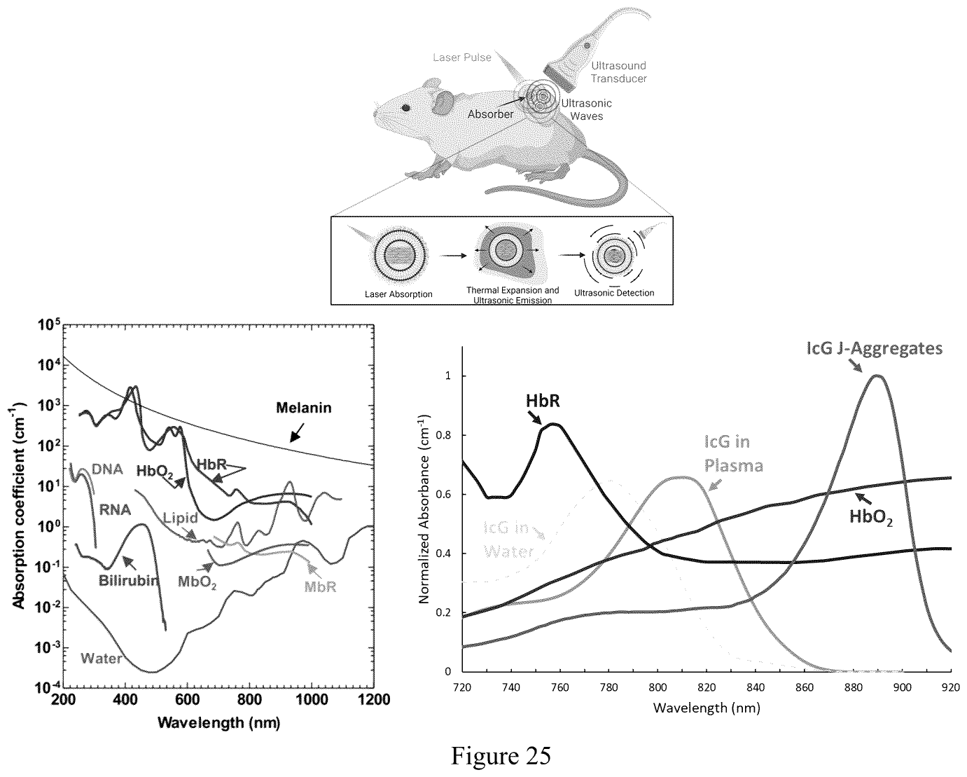

In vitro photoacoustic tests were performed using clear polyethylene tubing (Roboz Surgical Instruments, Gaithersburg, MD) embedded within tissue-mimicking polyacrylamide phantoms. Phantoms are created by following an approach similar to Hariri (Hariri, A. et al. Photoacoustics 22, 100245 (2021)). Briefly, a 12% w/v acrylamide solution is made by diluting a stock 19:1 acrylamide/bisacrylamide solution with deionized water and APS (0.08% w/v). Black ink (0.004% v/v) and TiO2 (0.08% w/v) are also added in to adjust optical properties (FIG. 25). This solution is degassed for 1 hour before mixing in TEMED (0.2% v/v). Once the TEMED is mixed in, the solution is rapidly poured into a plastic mold containing polyethylene tubing. The mold is lightly agitated as the gel is allowed to solidify and cool over the course of an hour.

All photoacoustic imaging was performed using the VEVO LAZR-X and F2 Systems (FUJIFILM VisualSonics, Toronto, Canada). The system was calibrated per manufacturer instructions prior to all experiments. Equal concentrations of IcG, IcG-JA, and L-JA were loaded into the plastic tubes before coupling the imaging phantom to the transducer with ultrasound gel. For base characterization, a full spectral scan from 680 nm to 970 nm was performed. Photostability measurements were obtained by imaging at either 800 nm or 890 nm for 30 minutes. At five-minute intervals, which equated to nearly every 1400 pulses (˜280 pulses per minute), the scan was paused to allow for a full spectral scan to be performed. RAW data files from the system were exported and were analyzed using custom software written in MATLAB.

### In Vitro Photothermal Heating

To assess the heating capacity of IcG and L-JA solutions, two different laser setups were utilized. All laser components including diodes, mounts, fiber optic cables, lens, and adapters were acquired from ThorLabs (Thorlabs Inc., Newton, NJ, USA) Two different laser diodes, one 808 nm the other 852 nm, were mounted, aligned, and focused in view of a high-resolution IR camera and tracked using accompanying software (Teledyne FLIR, Wilsonville, OR, USA). The 852 nm laser system included an optical isolator (ThorLabs) in the light path as recommended by the manufacturer, but otherwise the systems were identical. Laser power was calibrated to 1 W/cm² for both lasers and across all experiments. All experiments were conducted in 24- or 96-well plates filled with corresponding contrast agent solutions placed at the focus of the laser and IR camera.

### Cytotoxicity Assay

The cytotoxicity of L-JA, IcG-JA, and IcG was evaluated on HUVECs and HEK-293T cell lines. The HUVECs were cultured in MCDB-131 medium supplemented with 10% (v/v) fetal bovine serum, 1% L-glutamine, 1% penicillin-streptomycin, and 1% endothelial cell growth supplement. The HEK-293T cells were cultured in Dulbecco's modified Eagle medium (DMEM) supplemented with 10% (v/v) fetal bovine serum and 1% penicillin-streptomycin. Both cell lines were incubated at 37° C. with 5% CO₂ in a humidified atmosphere. The HUVEC and HEK-293T cells were seeded at the density of 10,000 cells per well and 5,000 cells per well in 96 well plates. After 24 hours, the culture media was replaced with fresh media containing L-JA, IcG-JA, and IcG at the concentration of 0.25 mM, 0.2 mM, 0.1 mM, and 0.05 mM. Cells incubated without particles were used as a control group. After 24 hours, the culture media was replaced with 1001 of fresh media. 20 μl of a mixture of [3-(4,5-dimethylthiazol-2-yl)-5-(3-carboxymethoxyphenyl)-2-(4-sulfophenyl)-2H-tetrazolium] (MTS) and phenazine ethosulfate (PES) was then added to each well. The cells were further incubated for 4 hours at 37° C. The absorbance signal at 490 nm was measured using a BioTek plate reader.

### In Vivo Imaging

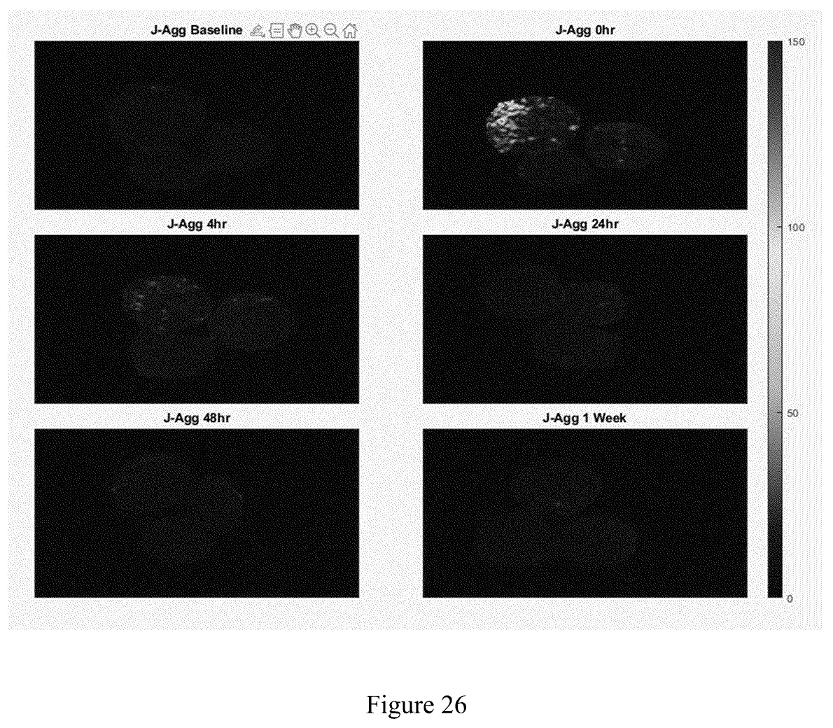

All in vivo experiments were conducted using protocols approved by the Institutional Animal Care and Use Committee at the University of Texas at Austin. All experiments were performed on albino 6-week old mice from Jackson Laboratory (Bar Harbor, ME). Prior to administration of anesthesia, all mice were treated with 100% oxygen for 2 minutes to ensure all mice had a similar baseline oxygenation. Mice were shaved and placed in a lateral recumbent position in the imaging system such that the transducer was positioned over the right kidney and renal artery. Isoflurane was maintained at 2% with 0.8 L/min oxygen flow rate through the duration of imaging. Before injection, baseline values for oxygenation and contrast agent signal are obtained. Ultrasound power-doppler imaging is also performed to ensure high blood flow and assist with the placement of ROIs. Oxygenation values were obtained using the VEVO system's “Oxyhemo” setting which alternates imaging at 750 nm and 850 nm and unmixes for HHb and HbO₂. Contrast agent signal was obtained by linear unmixing the signal from 710, 780, 830, 890, 900, and 920 nm imaging based on the PA spectra of IcG-JA (or IcG), HHb, and HbO₂. Unmixed signals for HHb and HbO₂ were also collected and compared with values obtained from “Oxyhemo” scans to ensure consistency. All presented values are the average of 130 frames for “Oxyhemo” imaging and 20 frames for multi-wavelength imaging. Following baseline imaging, a 5 mg/kg dose of L-JA (N=4 mice), IcG-JA (N=4 mice), or IcG (N=4 mice) were injected intravenously via the tail vein. All mice were imaged immediately after injection, with mice receiving liposomes also imaged at 4 hours, 24 hours, 48 hours, and 1 week after injection (FIG. 26). Mice receiving IcG-JA or IcG were imaged continuously for up to 30 minutes after injection. RAW data files from the system were exported and were analyzed using custom software written in MATLAB. Briefly, the scripts aggregate the data, calculate averages based on 3 drawn ROIs taken through all the frames, and plot the values obtained.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

We claim:

1. A composition comprising a lipid nanoparticle and a dye,
wherein the dye is a J-aggregate of the dye encapsulated in the lipid nanoparticle;
wherein the lipid nanoparticle:
a) comprises a high transition temperature lipid having a melting temperature of above about 60° C.;
b) has an absorption peak from about 850 nm to about 1100 nm wavelength; and
c) is amenable to an irradiation at a wavelength of at or above about 850 nm; and
wherein the composition is a transition metal-free composition.

2. The composition of claim 1, wherein the lipid nanoparticle has an absorption peak from about 885 nm to about 895 nm wavelength or is amenable to an irradiation at a wavelength of between about 885 nm to about 895 nm wavelength.

3. The composition of claim 1, wherein the high transition temperature lipid is selected from the group consisting of 1,2-diarachidoyl-sn-glycero-3-phosphocholine (20:0 PC), 1,2-dilauroyl-sn-glycero-3-phosphocholine (12:0 PC), 1,2-ditridecanoyl-sn-glycero-3-phosphocholine (13:0 PC), 1,2-dimyristoyl-sn-glycero-3-phosphocholine (14:0 PC), 1,2-dipentadecanoyl-sn-glycero-3-phosphocholine (15:0 PC), 1,2-dipalmitoyl-sn-glycero-3-phosphocholine (16:0 PC), 1,2-diheptadecanoyl-sn-glycero-3-phosphocholine (17:0 PC), 1,2-distearoyl-sn-glycero-3-phosphocholine (18:0 PC or DSPC), 1,2-dinonadecanoyl-sn-glycero-3-phosphocholine (19:0 PC), 1,2-diheneicosanoyl-sn-glycero-3-phosphocholine (21:0 PC), 1,2-dibehenoyl-sn-glycero-3-phosphocholine (22:0 PC), 1,2-ditricosanoyl-sn-glycero-3-phosphocholine (23:0 PC), 1,2-dilignoceroyl-sn-glycero-3-phosphocholine (24:0 PC), 1,2-diheptadecanoyl-sn-glycero-3-phospho-L-serine (17:0 PS), 1,2-distearoyl-sn-glycero-3-phospho-L-serine (18:0 PS), 1,2-dipalmitoyl-sn-glycero-3-phosphate (16:0 PA), 1,2-diheptadecanoyl-sn-glycero-3-phosphate (17:0 PA), 1,2-distearoyl-sn-glycero-3-phosphate (18:0 PA), 1,2-dipalmitoyl-sn-glycero-3-phosphoethanolamine (16:0 PE), 1,2-distearoyl-sn-glycero-3-phosphoethanolamine (18:0 PE), 1,2-diarachidoyl-sn-glycero-3-phosphoethanolamine (20:0 PE), and any combination thereof.

4. The composition of claim 1, wherein the J-aggregate of the dye is a J-aggregate of a cyanine dye or an indocyanine green J aggregate (ICGJ).

5. The composition of claim 1, wherein lipid nanoparticle has an average hydrodynamic diameter of about 100 nm to about 200 nm.

6. The composition of claim 1, wherein the lipid nanoparticle is a lipid vesicle or a liposome.

7. The composition of claim 1, wherein the composition further comprises a polymer.

8. The composition of claim 7, wherein the composition further comprises a second lipid.

9. The composition of claim 8, wherein the molar ratio of the high transition temperature lipid to the polymer to the second lipid is from about 80:30:10 to about 90:50:15.

10. The composition of claim 1, wherein the composition comprises a targeting domain attached to the surface of the lipid nanoparticle; and
wherein the targeting domain optionally binds to at least one cancer cell.

11. The composition of claim 10, wherein the targeting domain is selected from the group consisting of an antibody, an antibody fragment, a peptide sequence, aptamer, a ligand, a gene component, and any combination thereof.

12. The composition of claim 1, wherein the composition further comprises a therapeutic agent.

13. The composition of claim 12, wherein the therapeutic agent is a chemotherapeutic agent.

14. The composition of claim 1, wherein the composition is a contrast agent.

15. A method of generating the composition of claim 1, wherein the method comprises the steps of:
a) generating a lipid nanoparticle, wherein the lipid nanoparticle comprises a high transition temperature lipid having a melting temperature of above about 60° C.;
b) adding a dye;
c) encapsulating the dye in the lipid nanoparticle; and
d) heating the dye encapsulated in the lipid nanoparticle to generate a J-aggregate of the dye encapsulated by the lipid nanoparticle.

16. An imaging method, comprising the steps of:
contacting a biological tissue with the composition of claim 1,
applying energy to a biological tissue comprising the composition, and
imaging the biological tissue comprising the composition.

17. The method of claim 16, wherein imaging the biological tissue comprises application of an imaging technique selected from the group consisting of: photoacoustic imaging, thermal imaging, photothermal imaging, and any combination thereof.

18. A method of treating a disease or disorder in a subject in need thereof, wherein the method comprises the step of administering a therapeutically effective amount of the composition of claim 1 to the subject.

19. The method of claim 18, wherein the disease or disorder is a cancer.

20. A phototherapy method, comprising the steps of:
administering a therapeutically effective amount of the composition of claim 1 to the subject; and
irradiating the subject at a wavelength of at or above about 850 nm.
